(19) United States
(12) Patent Application Publication
(10) Pub. No.: US 2022/0122691 A1
(43) Pub. Date: Apr. 21, 2022

(54) HtrA Inhibitors and CagA Inhibitors and Use Thereof

(71) Applicant: PSOMAGEN INC., Rockville, MD (US)

(72) Inventors: Zachary Apte, San Francisco, CA (US); Jessica Richman, San Francisco, CA (US); Daniel Almonacid, San Francisco, CA (US); Valeria Marquez, San Francisco, CA (US)

(73) Assignee: PSOMAGEN INC., Rockville, MD (US)

(21) Appl. No.: 17/420,756

(22) PCT Filed: Jan. 6, 2020
(86) PCT No.: PCT/US2020/012347

§ 371 Date: Jul. 6, 2021

Related U.S. Application Data
(60) Provisional application No. 62/788,939, filed on Jan. 6, 2019.
(60) Provisional application No. 62/788,958, filed on Jan. 7, 2019.
(60) Provisional application No. 62/788,953, filed on Jan. 7, 2019.
(60) Provisional application No. 62/788,955, filed on Jan. 7, 2019.
(60) Provisional application No. 62/788,957, filed on Jan. 7, 2019.

(51) Int. Cl. G16B 15/30; C07C 317/14

(52) U.S. Cl. CPC G16B 15/30; C07C 317/14

(57) ABSTRACT

The present application relates to new HtrA inhibitors and use thereof. Additionally, the present application also relates to new peptides for inhibiting CagA and use thereof.

### CROSS-REFERENCE TO RELATED APPLICATIONS

This application claims the benefit of U.S. Provisional Patent Application No. 62/788,955 filed Jan. 7, 2019 entitled “Method and System to Target Bacteria with Antibacterial Compounds from the Microbiota”; U.S. Provisional Patent Application No. 62/788,957 filed Jan. 7, 2019 entitled “Method and System for Protecting Cell Junctions Proteins of Intestinal Epithelium”; U.S. Provisional Patent Application No. 62/788,958 filed Jan. 7, 2019 entitled “Method and System for Targeting Microbiota Proteins Mimicking Host Proteins”; U.S. Provisional Patent Application No. 62/788,939 filed Jan. 6, 2019 entitled “Small molecules as Inhibitors of HpHtRA protein in Helicobacter pylori”; U.S. Provisional Patent Application No. 62/788,953 filed Jan. 7, 2019 entitled “CagA inhibitors associated with the H. pylori and/or gastric cancer”, all of which are incorporated by reference herein in their entirety.

### BACKGROUND

Some antibacterial compounds produced by human microbiota are involved in different biological functions associated with human health and/or disease conditions. Among the most common antibacterial compounds can include lantibiotics, bacteriocins and microcins.

Bacteriocins and lantibiotics are antimicrobial peptides or proteins (between 20 and 60 amino acids) synthesized by bacteria that inhibit or kill other microorganisms. Antibacterial compounds can promote a bactericidal or bacteriostatic effect, inhibiting cell growth. Bacteriocins have been mainly used as safe food preservatives because they are easily digested by the human gastrointestinal tract. However, some bacteriocins and lantibiotics are used in health related applications. Subtilosin A from Bacillus subtilis show anti-viral and spermicidal activities. Nisin, which is produced by some Gram-positive bacteria including Lactococcus and Streptococcus species, has the ability to control many Gram-positive pathogens, such as Streptococcus pneumoniae, Enterococci and Clostridium difficile. Microcins are small peptides (less than 10 kDa) derived exclusively from Enterobacteriaceae and have a potent antibacterial activity against close-related bacteria that produce it. The action of microcin B17 on sensitive Escherichia coli cells leads to the arrest of DNA replication and, consequently, to the induction of the SOS response. Diverse applications of antibacterial compounds are studied because some of them are recognized as Generally Recognized as Safe (GRAS) compounds by the FDA.

Examples of the use of bacteriocins include:
Salivaricin A, a bacteriocin producing by Streptococcus salivarius K12 has been studied to inhibit malodour-associated bacterial species such as Streptococcus anginosis T29, Eubacterium saburreum and Micromonas micros.
Ruminococcin A, produced by Ruminococcus gnavus and Clostridium nexile has been studied against C. perfringens and C. difficile, suggesting as therapeutic agent against these pathogens. These pathogens are associated to antibiotic associated diarrhoea, and sporadic diarrhoea in humans.
Bacteriocin staphylococcus 188 has been studied against Newcastle disease virus, influenza virus.

It has been described that the mucosal epithelia is used by several microorganisms to adhere, internalize and/or take advantage of the host properties consequently producing diseases. As an example, one of the most targeted proteins is e-cadherin, a cell adhesion and tumor suppressor protein that has a key role in the prevention of cancer. As an example, microorganisms such as Escherichia coli, Shigella flexneri, Campylobacter jejuni and Helicobacter pylori express HtrA virulence factors, that trigger cleavage of an extracellular section of e-cadherin. E-cadherin cleavage leads to weaker cell-cell adhesion, which allow microorganisms to enter the intracellular epithelial space and provoke diseases, from diarrhea until cancer. Other microorganisms, such as Listeria monocytogenes, can be responsible for diseases such as gastroenteritis, meningoencephalitis and/or sepsis, produce cell wall internalins to bind e-cadherin and promote internalization into host cells. Thus, virulence factors that can bind e-cadherin are promising drug targets.

Gut microbiota includes a reservoir of microbes that are separated from the rest of the human system by intestinal epithelium. However, some conditions or diseases, such as intestine bowel disease (IBD), antibiotics use, aging, and/or other suitable conditions and/or diseases, etc., might provoke the intestinal mucosal barrier to become permeable to molecules produced by microbiota, in a phenomenon known as “leaky gut” or “permeable intestine”.

The molecular mimicry mechanism is one of several mechanisms (e.g., apart from the genetic predisposition), that can lead to autoimmunity. In that regard, bacteria can generate peptides that have a similar sequence than self peptides (e.g., molecular mimicry) and in a leaky gut environment, those peptides can be put into contact with T-cells and/or B-cells. In this way, T-cells and/or B-cells can cross react with the host epitopes, leading to autoimmunity. This phenomenon is possible because MHC class II molecules could be expressed on intestinal luminal cells, and those cells can process luminal peptides and present them to T-cells. Thus, bacterial peptides can lead to generate antibodies that can react against human proteins, causing the inhibition of those proteins functions. This generation of antibodies may eventually provoke and/or worsen metabolic, inflammatory and/or autoimmune diseases.

Some studies in mice have suggested that in genetically predisposed mice, the introduction of a gut microbiota species can trigger arthritis. Also, studies have shown that microbiota can participate in triggering other autoimmune diseases, such as Crohn's, Lupus, Rheumatoid Arthritis, and/or Psoriasis.

Helicobacter pylori has been classified as a class-I carcinogen responsible of gastric cancer by the World Health Organization. H. pylori is able to colonize gastric epithelial of host cells, altering gastric mucosa and provoking several inflammatory conditions, such as ulcers, chronic gastritis, and/or gastric cancer. Moreover, H. pylori has become resistant to antibiotics during the last decades.

One common target used by microorganisms for host cell attachment and invasion is through E-cadherin. In gastric epithelial cells, E-cadherin plays a key role in maintaining cell junctions, preventing bacterial invasions, and/or preventing cancer cell proliferation. E-cadherin can be described as a single transmembrane protein which has five extracellular domain, an intracellular domain and a transmembrane domain.

HtrA is a heat shock induced serine protease with homologs in several bacteria and eukaryotes. HtrA usually contributes to proteolytic degradation of abnormal proteins. HtrA proteins share common architecture such a proteolytic domain and a C-terminal PDZ domain involved in the binding of substrates. In H.pylori, HtrA has been shown to cleave the ectodomain of E-cadherin. Also, HtrA in other microorganisms such as Campylobacter jejuni is involved in bacterial invasion and cleavage of E-cadherin. Both C. jejuni and H. pylori actively secrete HtrA proteins to the extracellular environment, where they target host cell factors. Recently, the motif from E-cadherin cleaved by HtrA can be described as ([VITA]-[VITA]-x-x-D-[DN]).

Sequence alignments of several HtrA proteins have shown a conserved chymotrypsinlike proteolytic domain, including three important residues: HIS 116, ASP 147, and SER 221. HtrA proteins have shown some advantages as a target of drugs, for example including one or more of:

It is secreted towards the extracellular milieu or at the bacterial surface, which can then enable it to be accessible to drugs;

The enzymatic active site can be characterized and described;

Host factors targeted by HtrA proteins, such as E-cadherin, proteoglycans and fibronectin, have important functions in bacterial pathogenesis.

Deletion of htrA gene in bacteria has been found to be lethal. Selective inhibition of HtrA proteins may help antibiotic treatment by preventing bacterial access to gastrointestinal tissues

Gastric Cancer (GC) is the fifth most common cancer in the world (incidence 5.7% of all cancers), and the third leading cause of cancer deaths (8.2%), according with Global Cancer Observatory (http://gco.iarc.fr).

Additionally, Helicobacter pylori have been labeled as responsible for nearly 90% of the world's burden of noncardia gastric cancer, and it is the most relevant infectious agent associated with gastric cancer. At the same time, multiple secondary effects have been associated with gastric cancer and H. pylori, as B12 and iron deficiency, due to bad intestinal absorption, preeclampsia and also, due to failure in the effect of therapeutic drugs.

H. pylori Infection Mechanisms:

Multiple metabolic pathways from H.pylori have been associated to gastric cancer through gastric damage, those pathways include urease and invasive virulence proteins, such as cytotoxin-associated gene A (CagA), Binding Adhesin A (BabA), sialic acid-binding adhesin (SabA), Vacuolating cytotoxin A (VacA), outer inflammatory protein A (OipA). Particularly, CagA is a virulence factor protein described as one of the major inducer of GC, due to their capability to interact with multiple human proteins include of 3 domains well formed (I, II, III), a pathogenicity domain (EPIYA motif) and a C-terminal multimerization domain (CM). CagA pathogenicity compromise multiple cellular responses of the cell as motility, proliferation, mitosis, polarity, and junctions. Particularly the CM domain, have been associated particularly with an interaction with epithelial cadherin (e-cadherin), disrupting the Wnt pathway and junction proteins, one of the main paths associated with GC. The junctions proteins have been relevant due to their rol over the cohesion of the epithelium, regulating the cell morphology and rearrangement of the cellular cytoskeleton, and as such can include implications over several cancer types. Therefore, epithelial Cadherin (E-cadherin) is one of the most important proteins in mediating communication between cells, acting like an indicator of growth and proliferation chances for the cell, or growth by an intracellular way.

Under normal situations, E-cadherin binds with β-catenin as a normal part of Wnt signaling cascade (which is inactive). However, in gastric cancer, CagA binds and interact with E-cadherin which competes with β-catenin binding processes, which promote the accumulation of β-catenin, and which can promote the transcription of multiple factors involved in signaling of cellular proliferation, including possible oncogenic genes.

### BRIEF SUMMARY OF THE DISCLOSURE

The bacterial chaperone and serine protease-high temperature requirement A (HtrA)—is closely associated with the establishment and progression of several infectious diseases. The present disclosure relates to one or more HtrA inhibitors. Such inhibitors can be used as anti-infectious agents. For example, an HtrA inhibitor can comprise Formula (I):

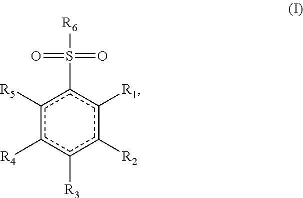

wherein:
R₁ is H, halo, cyano, OH, (C₁-C₆)alkyl, (C₃-C₁₀)heterocyclo, (C₃-C₁₀)cycloalkyl, aryl, or heteroaryl, any of which may be optionally substituted with 1 or more R^{w} groups as allowed by valence selected from the groups listed in Table 1;
R₂ is H, halo, cyano, OH, (C₁-C₆)alkyl, (C₂-C₈)alkenyl, (C₁-C₆)alkoxy, (C₃-C₁₀)heterocyclo, (C₃-C₁₀)cycloalkyl, aryl, or heteroaryl, any of which may be optionally substituted with 1 or more R^{w} groups as allowed by valence;
R₃ is H, halo, cyano, OH, (C₁-C₆)alkyl, (C₂-C₈)alkenyl, (C₂-C₈)carboxyalkyl; N—(C₁-C₆)alkylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence;
R₄ is H, halo, cyano, OH, (C₁-C₆)alkyl, (C₂-C₈)alkenyl, (C₂-C₈)carboxyalkyl; N—(C₁-C₆)alkylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence;
R₅ is H, halo, cyano, OH, (C₁-C₆)alkyl, (C₂-C₈)alkenyl, (C₂-C₈)carboxyalkyl; N—(C₁-C₆)alkylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence;
R₆ is H, halo, cyano, OH, (C₁-C₆)alkyl, (C₂-C₈)alkenyl, (C₂-C₈)carboxyalkyl; N—(C₁-C₆)alkylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence;

R^{w} at each occurrence is independently H, halo, cyano, nitro, oxo, amino, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl, wherein n is 0, 1, 2, 3, 4, 5, or 6.

In an embodiment, the HtrA inhibitor is selected from the group consisting of:
N′-benzyl-N-[2-[(2R)-1-(4-methylphenyl)sulfonylpiperidin-2-yl]ethyl]oxamide; 1-[2-[methyl(naphthalen-2-ylsulfonyl)amino]acetyl]piperidine-4-carboxamide; 8-(4-methylphenyl)sulfonyl-4-(2,4,6-trimethylphenyl)sulfonyl-1-oxa-4,8-diazaspiro[4.5]decane;
N-{1-[2-(2-Biphenylyloxy)ethyl]-3-pyrrolidinyl}-3,4-difluorobenzenesulfonamide; 1-(2-Anthrylsulfonyl)-3-piperidinecarboxylic acid; (3S)-1-Carbamimidoyl-N-({(2S)-1-[N-(2-naphthylsulfonyl)glycyl]-2-pyrrolidinyl}methyl)-3-piperidinecarboxamide;
(3S)-1-Carbamimidoyl-N-({(2S)-1-[N-(2-naphthylsulfonyl)-L-alanyl]-2-pyrrolidinyl}methyl)-3-piperidinecarboxamide;
cyclohexyl[4-(1-naphthylsulfonyl)-2-(trifluoromethyl)-1-piperazinyl]methanone; and 2-[(8S,11R)-11-((1R)-1-hydroxy-2-[(3-methylbutyl)(phenylsulfonyl)amino]ethyl)-6,9-dioxo-2-oxa-7,10-diazabicyclo[11.2.2]heptadeca-1(15),13,16-trien-8-yl]acetamide.

The present application also relates to a method of treating a bacterial infection comprising administering a pharmaceutically effective amount of any one or more of the HtrA inhibitor described herein to a human subject in need thereof. In an embodiment, the bacterial infection is a Helicobacter pylori infection.

The present disclosure also relates to a peptide of inhibiting CagA, a H. pylori virulence factor. Such peptides can be used as a therapeutic agent against the CagA mediated gastric cancer. In an embodiment, a peptide of inhibiting CagA has the sequence of X₁X₂X₃X₄X₅X₆X₇X₈X₉X₁₀X₁₁X₁₂; wherein:
X₁ is D, R, H, I, F, P, W, or Y;
X₂ is T or N;
X₃ is D, N, or Y;
X₄ is P, E, L, or Y;
X₅ is T, R, or L;
X₆ is A or S;
X₇ is P, R, E, I, or L;
X₈ is P, I, L, or W;
X₉ is F, or Y;
X₁₀ is D, F, or W;
X₁₁ is S, A, D, E, H, I, L, or Y; and
X₁₂ is L, A, N, W, or Y.

In some embodiments, a peptide of inhibiting CagA has the sequence of RTDPTAPPYDSL or DTDPTAPPYDSL.

The present discloser also relates to a method of treating gastric cancer comprising administering a pharmaceutically effective amount of the peptide described herein to a human subject in need thereof. Such peptides include, for example, DTDPTAPPYDSL and RTDPTAPPYDSL.

In another aspect, the present disclosure relates to a method of inhibiting, down-regulating, reducing and/or killing pathogenic bacteria comprising steps of: screening a microorganism that produces an antibacterial compound; conducting structural analysis of the antibacterial compound; and modifying the antibacterial compound to improve the affinity to target bacteria.

### BRIEF DESCRIPTION OF THE DRAWINGS

FIG. 1 schematically illustrates a method and/or system to detect new antibacterial compounds produced by microbiota bacteria.

FIG. 2 schematically illustrates a method and/or system to to modify the antibacterial compounds.

FIG. 3 schematically illustrates a method and/or system to detect new bacterial virulence factors that alter cell junctions.

FIG. 4 schematically illustrates a method and/or system to generate peptide inhibitors against virulence factors, using the specific example of e-cadherin as cell-junction protein.

FIG. 5 schematically illustrates a method and/or system to find candidate bacterial proteins, peptides, and/or other suitable components that can trigger autoimmune response.

FIG. 6 illustrates Ro60 antigen orthologue protein found in Bacteroides thetaiotaomicron associated with lupus, including the MHC-class II binding zone and the RNA zone.

FIG. 7 illustrates homology model of trimeric HtrA from Helicobacter pylori (left), and catalytic site of the protease, depicting residues HIS 116, ASP 147 and SER 221 (right).

FIG. 8 illustrates specific examples of selected candidate molecules with docking energy of binding >−9.5 kcal/mol against HtrA receptor.

FIG. 9. Illustrates specific examples of selected candidate molecules with docking energy of binding >−8.9 kcal/mol against HtrA receptor.

FIG. 10. Illustrates specific examples of selected candidate molecules with docking energy of binding >−8.9 kcal/mol against HtrA receptor.

FIG. 11 illustrates the estimated deaths of cancer patients by type.

FIG. 12 illustrates the number of estimated cancers caused by the bacteria infection.

### DETAILED DESCRIPTION OF THE DISCLOSURE

### Definition

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

“ [symbol] ” indicates the double bond in E or Z configuration.

The term “H” denotes a single hydrogen atom. This radical may be attached, for example, to an oxygen atom to form a hydroxyl radical.

Where the term “alkyl” is used, either alone or within other terms such as “haloalkyl” or “alkylamino”, it embraces linear or branched radicals having one to about twelve carbon atoms. More preferred alkyl radicals are “lower alkyl” radicals having one to about six carbon atoms. Examples of such radicals include methyl, ethyl, n-propyl, isopropyl, n-butyl, isobutyl, sec-butyl, tert-butyl, pentyl, isoamyl, hexyl and the like. Even more preferred are lower alkyl radicals having one or two carbon atoms. The term “alkylenyl” or “alkylene” embraces bridging divalent alkyl radicals such as methylenyl or ethylenyl. The term “lower alkyl substituted with R²” does not include an acetal moiety. The term “alkyl” further includes alkyl radicals wherein one or more carbon atoms in the chain is substituted with a heteroatom selected from oxygen, nitrogen, or sulfur.

The term “alkenyl” embraces linear or branched radicals having at least one carbon-carbon double bond of two to about twelve carbon atoms. More preferred alkenyl radicals are “lower alkenyl” radicals having two to about six carbon atoms. Most preferred lower alkenyl radicals are radicals having two to about four carbon atoms. Examples of alkenyl radicals include ethenyl, propenyl, allyl, propenyl, butenyl and 4-methylbutenyl. The terms “alkenyl” and “lower alkenyl”, embrace radicals having “cis” and “trans” orientations, or alternatively, “E” and “Z” orientations.

The term “alkynyl” denotes linear or branched radicals having at least one carbon-carbon triple bond and having two to about twelve carbon atoms. More preferred alkynyl radicals are “lower alkynyl” radicals having two to about six carbon atoms. Most preferred are lower alkynyl radicals having two to about four carbon atoms. Examples of such radicals include propargyl, and butynyl, and the like.

Alkyl, alkylenyl, alkenyl, and alkynyl radicals may be optionally substituted with one or more functional groups such as halo, hydroxy, nitro, amino, cyano, haloalkyl, aryl, heteroaryl, and heterocyclo and the like.

The term “halo” means halogens such as fluorine, chlorine, bromine or iodine atoms.

The term “haloalkyl” embraces radicals wherein any one or more of the alkyl carbon atoms is substituted with halo as defined above. Specifically embraced are monohaloalkyl, dihaloalkyl and polyhaloalkyl radicals including perhaloalkyl. A monohaloalkyl radical, for example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1 to 6 carbon atoms. Even more preferred are lower haloalkyl radicals having one to three carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “perfluoroalkyl” means alkyl radicals having all hydrogen atoms replaced with fluoro atoms. Examples include trifluoromethyl and pentafluoroethyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. Even more preferred are lower hydroxyalkyl radicals having one to three carbon atoms.

The term “alkoxy” embraces linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. Even more preferred are lower alkoxy radicals having one to three carbon atoms. Alkoxy radicals may be further substituted with one or more halo atoms, such as fluoro, chloro or bromo, to provide “haloalkoxy” radicals. Even more preferred are lower haloalkoxy radicals having one to three carbon atoms. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one or two rings, wherein such rings may be attached together in a fused manner. The term “aryl” embraces aromatic radicals such as phenyl, naphthyl, indenyl, tetrahydronaphthyl, and indanyl. More preferred aryl is phenyl. An “aryl” group may have 1 or more substituents such as lower alkyl, hydroxyl, halo, haloalkyl, nitro, cyano, alkoxy, and lower alkylamino, and the like. Phenyl substituted with —O—CH₂—O— forms the aryl benzodioxolyl substituent.

The term “heterocyclyl” (or “heterocyclo”) embraces saturated, partially saturated and unsaturated heteroatom-containing ring radicals, where the heteroatoms may be selected from nitrogen, sulfur and oxygen. It does not include rings containing —O—O—,—O—S— or —S—S— portions. The “heterocyclyl” group may have 1 to 4 substituents such as hydroxyl, Boc, halo, haloalkyl, cyano, lower alkyl, lower aralkyl, oxo, lower alkoxy, amino and lower alkylamino.

Examples of saturated heterocyclic radicals include saturated 3 to 6-membered heteromonocyclic groups containing 1 to 4 nitrogen atoms [e.g., pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include dihydrothienyl, dihydropyranyl, dihydrofuryl and dihydrothiazolyl.

Examples of unsaturated heterocyclic radicals, also termed “heteroaryl” radicals, include unsaturated 5 to 6 membered heteromonocyclyl group containing 1 to 4 nitrogen atoms, for example, pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5- to 6-membered heteromonocyclic group containing an oxygen atom, for example, pyranyl, 2-furyl, 3-furyl, etc.; unsaturated 5 to 6-membered heteromonocyclic group containing a sulfur atom, for example, 2-thienyl, 3-thienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl].

The term heterocyclyl, (or heterocyclo) also embraces radicals where heterocyclic radicals are fused/condensed with aryl radicals: unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo [1,5-b]pyridazinyl]; unsaturated condensed heterocyclic group containing 1 to 2 oxygen atoms and 1 to 3 nitrogen atoms [e.g. benzoxazolyl, benzoxadiazolyl]; unsaturated condensed heterocyclic group containing 1 to 2 sulfur atoms and 1 to 3 nitrogen atoms [e.g., benzothiazolyl, benzothiadiazolyl]; and saturated, partially unsaturated and unsaturated condensed heterocyclic group containing 1 to 2 oxygen or sulfur atoms [e.g. benzofuryl, benzothienyl, 2,3-dihydro-benzo[1,4]dioxinyl and dihydrobenzofuryl]. Preferred heterocyclic radicals include five to ten membered fused or unfused radicals. More preferred examples of heteroaryl radicals include quinolyl, isoquinolyl, imidazolyl, pyridyl, thienyl, thiazolyl, oxazolyl, furyl and pyrazinyl. Other preferred heteroaryl radicals are 5- or 6-membered heteroaryl, containing one or two heteroatoms selected from sulfur, nitrogen and oxygen, selected from thienyl, furyl, pyrrolyl, indazolyl, pyrazolyl, oxazolyl, triazolyl, imidazolyl, pyrazolyl, isoxazolyl, isothiazolyl, pyridyl, piperidinyl and pyrazinyl.

Particular examples of non-nitrogen containing heteroaryl include pyranyl, 2-furyl, 3-furyl, 2-thienyl, 3-thienyl, benzofuryl, and benzothienyl, and the like.

Particular examples of partially saturated and saturated heterocyclyl include pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl, and the like.

The term “heterocyclo” thus encompasses the following ring systems:

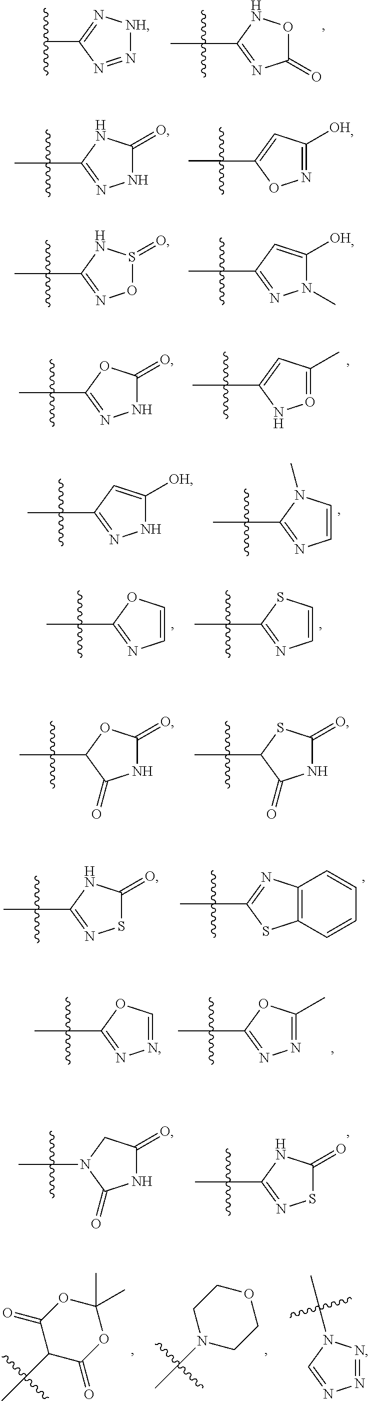

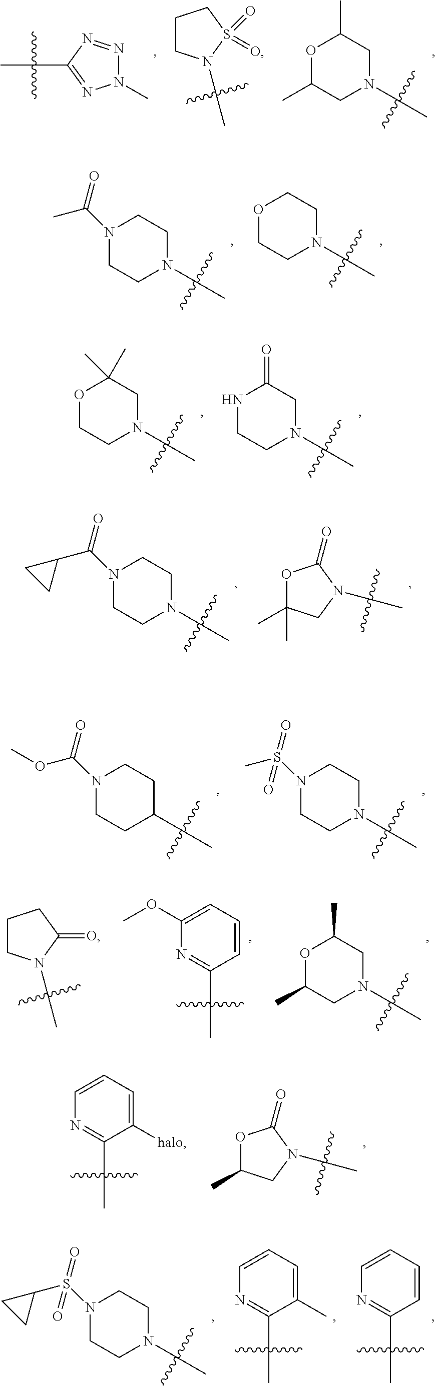

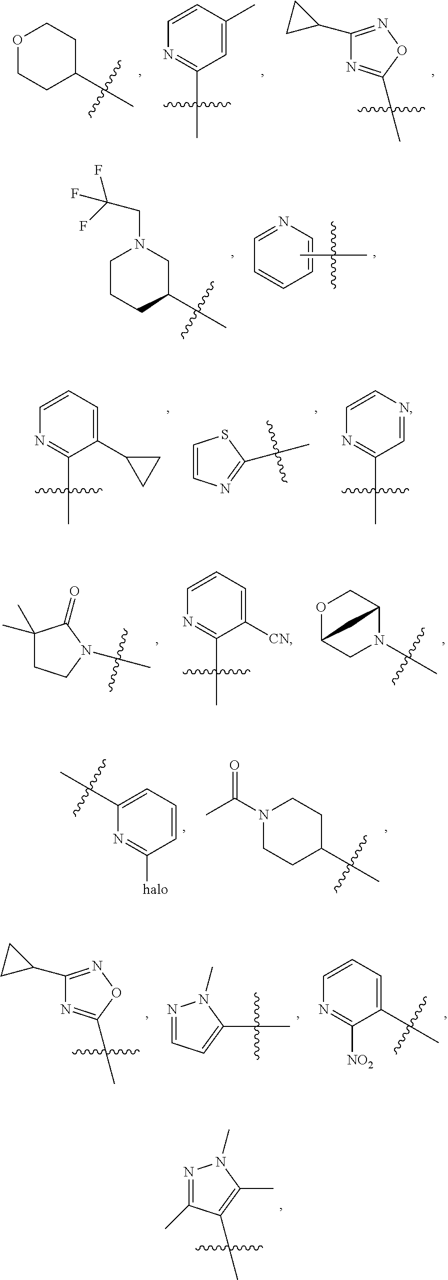

and the like.

The term “sulfonyl”, whether used alone or linked to other terms such as alkylsulfonyl, denotes respectively divalent radicals —SO₂—.

The terms “sulfamyl,” “aminosulfonyl” and “sulfonamidyl,” denotes a sulfonyl radical substituted with an amine radical, forming a sulfonamide (—SO₂NH₂).

The term “alkylaminosulfonyl” includes “N-alkylaminosulfonyl” where sulfamyl radicals are independently substituted with one or two alkyl radical(s). More preferred alkylaminosulfonyl radicals are “lower alkylaminosulfonyl” radicals having one to six carbon atoms. Even more preferred are lower alkylaminosulfonyl radicals having one to three carbon atoms. Examples of such lower alkylaminosulfonyl radicals include N-methylaminosulfonyl, and N-ethylaminosulfonyl.

The terms “carboxy” or “carboxyl,” whether used alone or with other terms, such as “carboxyalkyl,” denotes —CO₂H.

The term “carbonyl,” whether used alone or with other terms, such as “aminocarbonyl,” denotes —(C═O)—.

The term “aminocarbonyl” denotes an amide group of the formula C(═O)NH₂.

The terms “N-alkylaminocarbonyl” and “N,N-dialkylaminocarbonyl” denote aminocarbonyl radicals independently substituted with one or two alkyl radicals, respectively. More preferred are “lower alkylaminocarbonyl” having lower alkyl radicals as described above attached to an aminocarbonyl radical.

The terms “N-arylaminocarbonyl” and “N-alkyl-N-arylaminocarbonyl” denote aminocarbonyl radicals substituted, respectively, with one aryl radical, or one alkyl and one aryl radical.

The terms “heterocyclylalkylenyl” and “heterocyclylalkyl” embrace heterocyclic-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Even more preferred are lower heteroarylalkylenyl radicals having alkyl portions of one to three carbon atoms. Examples include such radicals as pyridylmethyl and thienylmethyl.

The term “aralkyl” embraces aryl-substituted alkyl radicals. Preferable aralkyl radicals are “lower aralkyl” radicals having aryl radicals attached to alkyl radicals having one to six carbon atoms. Even more preferred are “phenylalkylenyl” attached to alkyl portions having one to three carbon atoms. Examples of such radicals include benzyl, diphenylmethyl and phenylethyl. The aryl in said aralkyl may be additionally substituted with halo, alkyl, alkoxy, halkoalkyl and haloalkoxy.

The term “alkylthio” embraces radicals containing a linear or branched alkyl radical, of one to ten carbon atoms, attached to a divalent sulfur atom. Even more preferred are lower alkylthio radicals having one to three carbon atoms. An example of “alkylthio” is methylthio, (CH₃S—).

The term “haloalkylthio” embraces radicals containing a haloalkyl radical, of one to ten carbon atoms, attached to a divalent sulfur atom. Even more preferred are lower haloalkylthio radicals having one to three carbon atoms. An example of “haloalkylthio” is trifluoromethylthio.

The term “alkylamino” embraces “N-alkylamino” and “N,N-dialkylamino” where amino groups are independently substituted with one alkyl radical and with two alkyl radicals, respectively. More preferred alkylamino radicals are “lower alkylamino” radicals having one or two alkyl radicals of one to six carbon atoms, attached to a nitrogen atom. Even more preferred are lower alkylamino radicals having one to three carbon atoms. Suitable alkylamino radicals may be mono or dialkylamino such as N-methylamino, N-ethylamino, N,N-dimethylamino, and N,N-diethylamino, and the like.

The term “arylamino” denotes amino groups, which have been substituted with one or two aryl radicals, such as N-phenylamino. The arylamino radicals may be further substituted on the aryl ring portion of the radical.

The term “heteroarylamino” denotes amino groups, which have been substituted with one or two heteroaryl radicals, such as N-thienylamino. The “heteroarylamino” radicals may be further substituted on the heteroaryl ring portion of the radical.

The term “aralkylamino” denotes amino groups, which have been substituted with one or two aralkyl radicals. More preferred are phenyl-C₁-C₃-alkylamino radicals, such as N-benzylamino. The aralkylamino radicals may be further substituted on the aryl ring portion.

The terms “N-alkyl-N-arylamino” and “N-aralkyl-N-alkylamino” denote amino groups, which have been independently substituted with one aralkyl and one alkyl radical, or one aryl and one alkyl radical, respectively, to an amino group.

The term “aminoalkyl” embraces linear or branched alkyl radicals having one to about ten carbon atoms any one of which may be substituted with one or more amino radicals. More preferred aminoalkyl radicals are “lower aminoalkyl” radicals having one to six carbon atoms and one or more amino radicals. Examples of such radicals include aminomethyl, aminoethyl, aminopropyl, aminobutyl and aminohexyl. Even more preferred are lower aminoalkyl radicals having one to three carbon atoms.

The term “alkylaminoalkyl” embraces alkyl radicals substituted with alkylamino radicals. More preferred alkylaminoalkyl radicals are “lower alkylaminoalkyl” radicals having alkyl radicals of one to six carbon atoms. Even more preferred are lower alkylaminoalkyl radicals having alkyl radicals of one to three carbon atoms. Suitable alkylaminoalkyl radicals may be mono or dialkyl substituted, such as N-methylaminomethyl, N,N-dimethyl-aminoethyl, and N,N-diethylaminomethyl, and the like.

The term “alkylaminoalkoxy” embraces alkoxy radicals substituted with alkylamino radicals. More preferred alkylaminoalkoxy radicals are “lower alkylaminoalkoxy” radicals having alkoxy radicals of one to six carbon atoms. Even more preferred are lower alkylaminoalkoxy radicals having alkyl radicals of one to three carbon atoms. Suitable alkylaminoalkoxy radicals may be mono or dialkyl substituted, such as N-methylaminoethoxy, N,N-dimethylaminoethoxy, and N,N-diethylaminoethoxy, and the like.

The term “alkylaminoalkoxyalkoxy” embraces alkoxy radicals substituted with alkylaminoalkoxy radicals. More preferred alkylaminoalkoxyalkoxy radicals are “lower alkylaminoalkoxyalkoxy” radicals having alkoxy radicals of one to six carbon atoms. Even more preferred are lower alkylaminoalkoxyalkoxy radicals having alkyl radicals of one to three carbon atoms. Suitable alkylaminoalkoxyalkoxy radicals may be mono or dialkyl substituted, such as N-methylaminomethoxyethoxy, N-methylaminoethoxyethoxy, N,N-dimethylaminoethoxyethoxy, and N,N-diethylaminomethoxymethoxy, and the like.

The term “carboxyalkyl” embraces linear or branched alkyl radicals having one to about ten carbon atoms any one of which may be substituted with one or more carboxy radicals. More preferred carboxyalkyl radicals are “lower carboxyalkyl” radicals having one to six carbon atoms and one carboxy radical. Examples of such radicals include carboxymethyl, and carboxypropyl, and the like. Even more preferred are lower carboxyalkyl radicals having one to three CH₂ groups.

The term “halosulfonyl” embraces sulfonyl radicals substituted with a halogen radical. Examples of such halosulfonyl radicals include chlorosulfonyl and fluorosulfonyl.

The term “arylthio” embraces aryl radicals of six to ten carbon atoms, attached to a divalent sulfur atom. An example of “arylthio” is phenylthio.

The term “aralkylthio” embraces aralkyl radicals as described above, attached to a divalent sulfur atom. More preferred are phenyl-C₁-C₃-alkylthio radicals. An example of “aralkylthio” is benzylthio.

The term “aryloxy” embraces optionally substituted aryl radicals, as defined above, attached to an oxygen atom. Examples of such radicals include phenoxy.

The term “aralkoxy” embraces oxy-containing aralkyl radicals attached through an oxygen atom to other radicals. More preferred aralkoxy radicals are “lower aralkoxy” radicals having optionally substituted phenyl radicals attached to lower alkoxy radical as described above.

The term “heteroaryloxy” embraces optionally substituted heteroaryl radicals, as defined above, attached to an oxygen atom.

The term “heteroarylalkoxy” embraces oxy-containing heteroarylalkyl radicals attached through an oxygen atom to other radicals. More preferred heteroarylalkoxy radicals are “lower heteroarylalkoxy” radicals having optionally substituted heteroaryl radicals attached to lower alkoxy radical as described above.

The term “cycloalkyl” includes saturated carbocyclic groups. Preferred cycloalkyl groups include C₃-C₆ rings. More preferred compounds include, cyclopentyl, cyclopropyl, and cyclohexyl.

The term “cycloalkylalkyl” embraces cycloalkyl-substituted alkyl radicals. Preferable cycloalkylalkyl radicals are “lower cycloalkylalkyl” radicals having cycloalkyl radicals attached to alkyl radicals having one to six carbon atoms. Even more preferred are “5 to 6-membered cycloalkylalkyl” attached to alkyl portions having one to three carbon atoms. Examples of such radicals include cyclohexylmethyl. The cycloalkyl in said radicals may be additionally substituted with halo, alkyl, alkoxy and hydroxy.

The term “cycloalkenyl” includes carbocyclic groups having one or more carbon-carbon double bonds including “cycloalkyldienyl” compounds. Preferred cycloalkenyl groups include C₃-C₆ rings. More preferred compounds include, for example, cyclopentenyl, cyclopentadienyl, cyclohexenyl and cycloheptadienyl.

The term “comprising” is meant to be open ended, including the indicated component but not excluding other elements.

A group or atom that replaces a hydrogen atom is also called a substituent.

Any particular molecule or group can have one or more substituent depending on the number of hydrogen atoms that can be replaced.

The symbol “-” represents a covalent bond and can also be used in a radical group to indicate the point of attachment to another group. In chemical structures, the symbol is commonly used to represent a methyl group in a molecule.

The term “therapeutically effective amount” means an amount of a compound that ameliorates, attenuates or eliminates one or more symptom of a particular disease or condition, or prevents or delays the onset of one of more symptom of a particular disease or condition.

The terms “patient” and “subject” may be used interchangeably and mean animals, such as dogs, cats, cows, horses, sheep and humans. Particular patients are mammals. The term patient includes males and females.

The term “pharmaceutically acceptable” means that the referenced substance, such as a compound of Formula I, or a salt of a compound of Formula I, or a formulation containing a compound of Formula I, or a particular excipient, are suitable for administration to a patient.

The terms “treating”, “treat” or “treatment” and the like include preventative (e.g., prophylactic) and palliative treatment.

The term “excipient” means any pharmaceutically acceptable additive, carrier, diluent, adjuvant, or other ingredient, other than the active pharmaceutical ingredient (API), which is typically included for formulation and/or administration to a patient.

The term “cancer” means a physiological condition in mammals that is characterized by unregulated cell growth. General classes of cancers include carcinomas, lymphomas, sarcomas, and blastomas.

The following description of the embodiments is not intended to limit the embodiments, but rather to enable any person skilled in the art to make and use.

In embodiments, methods (e.g., pipeline, etc.) and/or systems (e.g., components facilitating performance of the pipeline; therapeutic compositions etc.) (and/or suitable portions of the embodiments) can include and/or function to inhibit, down-regulate, reduce and/or kill pathogenic bacteria using antibacterial compounds from the microbiota. In embodiments, the methods and/or systems can use one or more bioinformatics pipelines to identify compounds in the microbiota and/or can use one or more structural biology techniques to design new antibacterial compounds, such as by using as a basis the existing ones. In examples, the obtained antibacterial compounds can be used as treatment of one or more diseases and/or conditions, such as for healthcare, biotechnology, and pharmaceutical applications. In examples, other uses of these antibacterial compounds can additionally or alternatively include one or more of: food preservation, producing active probiotic culture, treatment of infections, antibiotic resistance to conventional antibiotics, post-surgical control of infectious bacteria, anti-cancer agents, and/or other suitable uses.

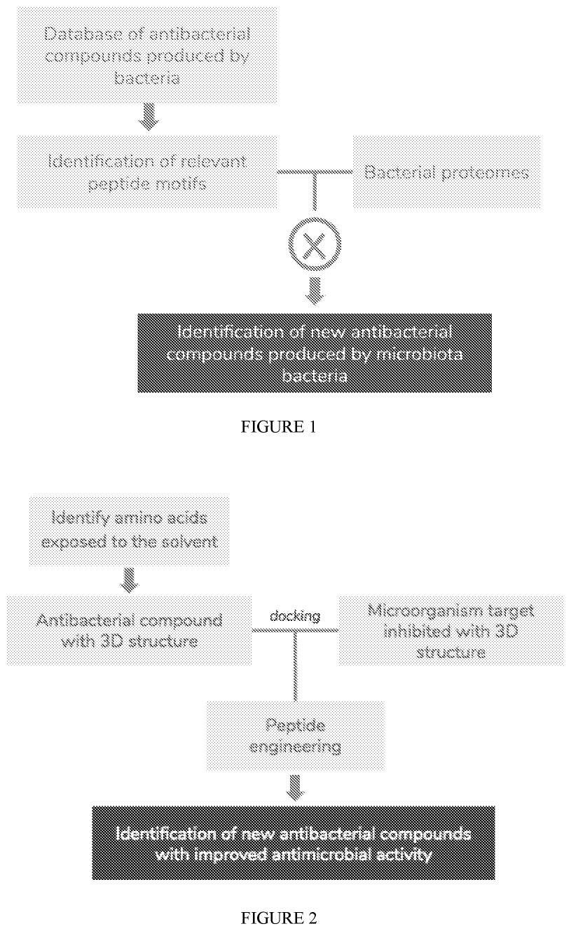

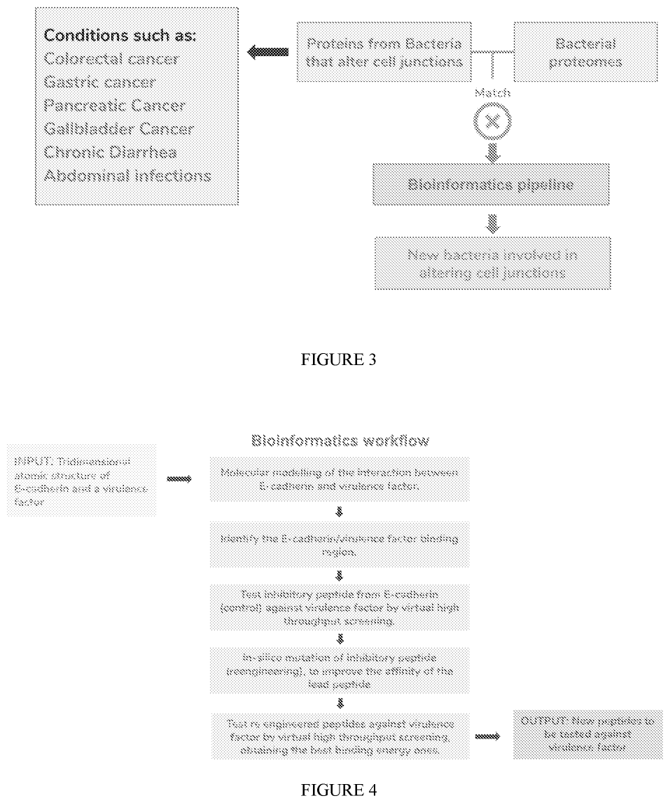

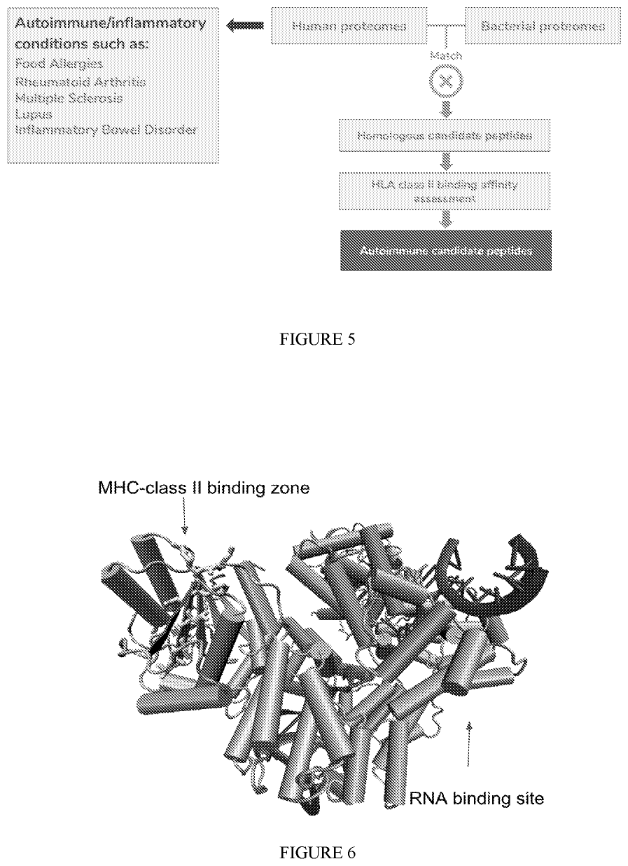

In embodiments, the method (e.g., pipeline, etc.) and/or system can include a first stage (and/or can be performed at any suitable time and frequency), which can include finding new antibacterial compounds produced by the microbiota. In examples, in this stage (and/or at any suitable time and frequency), a screening of known antibacterial compounds-producing microorganisms and/or antibacterial compounds can be performed, such as to generate one or more databases of antibacterial compounds produced by bacteria and/or other suitable microorganisms. In examples, all related information can be determined and/or stored (e.g., data usable for subsequent steps, etc.), such as including one or more of: the name of the antibacterial, the microorganisms that produce it, the application, host site, target microorganisms that inhibit and/or kill, and/or any other suitable data. In examples, curated antibacterial compounds (e.g., lantibiotic, bacteriocin, microcin, etc.), such as stored in the database, can be search against reference proteomes (e.g., from Uniprot or NCBI databases, etc.) using one or more sequence alignment algorithms (e.g., BLAST, FASTA, Clustal, among others, etc.). In examples, the alignment(s) can be used to identify peptide motif(s) that can be useful to predict the binding region of antibacterial compounds to other microorganisms, and/or to identify new bacteria-producing antibacterial compounds (e.g., an example of this stage is depicted in FIG. 1).

In embodiments, the method (e.g., pipeline, etc.) and/or system can include second stage (and/or can be performed at any suitable time and frequency) which can include and/or function to allow modification of the antibacterial compounds to improve the antimicrobial activity. In examples, the method and/or system can include modifying antibacterial peptides that have a defined tridimensional structure and/or have a known particular target (e.g., obtained from a structural database, such as Protein Data Bank, Bactibase, BAGEL, among others, etc.). In examples, according to that, and/or based on the identification of relevant peptide motif(s) from the first stage (and/or at any suitable time and frequency), a structural analysis can be performed to identify whether those motifs are exposed to the solvent and, therefore, can interact with proteins from other microorganisms. In examples, this analysis can be performed using solvent-accessible surface area (SASA) but can additionally or alternatively be otherwise performed. In examples, then, a molecular docking (e.g., as control) can be performed to model the atomic interaction between the antimicrobial peptide and/or motif and the target from a microorganism(s) known to be inhibited by the action of the antibacterial peptide. In examples, both molecules are considered rigid, that is, the bonds do not rotate and maintain the secondary structure. In examples, taking this into account, new antimicrobial peptides can be designed. In examples, to do this, modifications on segments of amino acids of antibacterial peptide(s) can be performed to determine and/or obtain new antibacterial peptide(s) with improved antimicrobial activity. The modifications can include mutating each position of peptides (and/or any suitable position) for one or more of the remaining 19 amino acids. In examples, subsequently, docking between modified peptides and the target can be performed. In examples, thus, the new antibacterial peptide can bind with high affinity to the target, and therefore, can improve their antimicrobial activity (e.g., an example of this procedure is shown in FIG. 2).

Embodiments can additionally or alternatively include applying any suitable approaches described herein for identification, generation, application, provision, and/or other suitable usage (e.g., in therapeutic compositions, etc.) of any suitable peptides, proteins, and/or other components, such as for any suitable antimicrobial activity, diseases, and/or conditions (e.g., described herein, etc.).

Embodiments of the method and/or system can additionally or alternatively include:

One or more methodologies to identify new antibacterial compounds (e.g., peptides, proteins, etc.) produced by the human microbiota.

One or more methodologies to modify the antibacterial compounds (e.g., peptides, proteins, etc.) to determine and/or obtain new ones.

Embodiments can additionally or alternatively include applying any suitable approaches described herein for identification, generation, application, provision, and/or other suitable usage (e.g., in therapeutic compositions, etc.) of any suitable peptides, proteins, and/or other components, such as for any suitable antimicrobial activity, diseases, and/or conditions (e.g., described herein, etc.).

In embodiments, methods (e.g., pipeline, etc.) and/or systems (e.g., components facilitating performance of the pipeline; therapeutic compositions etc.) (and/or suitable portions of the embodiments) can function to and/or include identification and/or targeting of virulence factors in bacteria (and/or other suitable microorganisms) that bind human cell-junctions proteins (e.g., e-cadherin, etc.), such as new targets of drugs that can help to prevent diseases and/or conditions provoked by those bacteria (and/or other suitable microorganisms), such as one or more of: colorectal cancer, gastric cancer, pancreatic cancer, gallbladder cancer, chronic diarrhea, abdominal infections and/or other suitable diseases and/or conditions. Additionally or alternatively, embodiments of the method and/or system can include the protection of cell-junctions proteins from cleavage mediated by the binding of bacterial virulence factors. In examples, the protection is addressed through the development of new peptide drugs. Additionally or alternatively, embodiments can include a pipeline and/or suitable approaches, which allow identification of new virulence proteins that target cell-junctions proteins. In examples, by analyzing the binding interface between the proteins, new peptides that can target virulence factor can be generated, aimed to prevent cell-junction protein binding and/or cleavage. In examples, the method and/or system can include and/or otherwise be used for new drugs that can prevent, ameliorate, and/or otherwise improve diseases provoked by adherens proteins from bacteria and/or other suitable microorganisms.

In embodiments, the method (e.g., pipeline) and/or system aims to find orthologous bacterial virulence factors to those already known by sequence matching against reference proteomes (e.g. available in NCBI). In examples, to this end, one or more alignment algorithms can be used (e.g., BLAST, FASTA, CLUSTAL, among others). Additionally or alternatively, structural information of known virulence factors (e.g., as those available in the Protein Data Bank—PDB) and predicted binding to a cell-junction protein (e.g e-cadherin) can be used to identify protein motifs in the binding site, allowing to identify new virulence factors in available bacterial proteomes. In examples, sequence similarity networks can be used to classify different classes of virulence factors that bind cell-junction proteins (e.g., E-cadherin, etc.), depending on the mechanisms that the proteins use to disrupt cell-junction proteins (e.g., E-cadherin, etc.).

In examples, new virulence factors that can alter cell junctions can additionally or alternatively be identified. Additionally or alternatively, using the available structural information in the structural databases (e.g., PDB, etc.), the binding site between the cell-junction protein (e.g., E-cadherin) and the different virulence factors can be determined. In examples, if a specific virulence factor is not found, a homology model of the structure can be obtained and the binding site can be found. In examples, once the binding site is determined, a peptide with higher affinity than the original cell-junction protein-binding site can be obtained by in-silico reengineering techniques (e.g., one or more of molecular docking, fragment-based discovery, free energy calculations, etc). In examples, thus, it is expected that new peptide drugs can bind with high affinity to the virulence factor, inhibiting by competition the original binding with the cell-junction protein.

However, any suitable portions, approaches, and/or steps described above and/or herein can be performed in any suitable sequence, and at any suitable time and frequency.

Embodiments of the method and/or system can additionally or alternatively include:

One or more pipelines to identify new proteins, peptides, and/or other components as virulence factors that can alter cell junctions, such as by cell-junction protein binding.

One or more pipelines to identify peptides, proteins, and/or other suitable components as inhibitors of the cell-junction protein (e.g., E-cadherin, etc.)/virulence factors binding.

Embodiments can additionally or alternatively include applying any suitable approaches described herein for identification, generation, application, provision, and/or other suitable usage (e.g., in therapeutic compositions, etc.) of any suitable proteins, peptides, and/or other components for targeting cell junctions, such as for any suitable conditions (e.g., described herein, etc.).

In embodiments, methods method (e.g., pipeline, etc.) and/or systems (e.g., components facilitating performance of the pipeline; therapeutic compositions etc.) (and/or suitable portions of the embodiments) disclosed can function to identify one or more bacterial proteins, peptides, and/or other components, that can cause cross reaction with human proteins, peptides, and/or other components. Additionally or alternatively, embodiments can include one or more approaches to inhibit the action of such bacterial proteins, peptides, and/or other components (e.g., for inhibiting the cross reaction with human proteins, peptides, and/or other components).

Embodiments of methods and/or systems (e.g., therapeutic compositions; etc.) can be identify bacterial proteins that can produce cross-reaction with human ones and to target such bacterial proteins using small molecules or peptides. In embodiments, the methods can include a procedure for identifying bacterial proteins that can lead to cross-reaction with host proteins. In examples, the obtained bacterial proteins are screened to find MHC class II epitopes, thus the proteins having those epitopes can be identified to generate antibody production. Additionally or alternatively, identified proteins can be new targets for the design of peptide inhibitors. In specific examples, new peptide-based drugs to target cross-reactive proteins can be used to alleviate or prevent the triggering of autoimmune diseases.

In embodiments, the method (e.g., pipeline, etc.) and/or system can include a first step (and/or can be performed at any suitable time and frequency), which can include one or more sequence identity searches performed between human gut microbiota reference proteomes (e.g., Uniprot and/or NCBI, etc.) against the human proteome and/or other suitable components. Any suitable combination of the organisms (e.g., taxa, all organisms, etc.) detected and/or detectable in the human gut (e.g., by any suitable database) can be considered, but any suitable database (e.g., Human Microbiome project, etc.) can additionally or alternatively be used. In a specific example, the similarity search is performed by using a sequence alignment algorithm (e.g. pBLAST), but any suitable similarity search approaches can additionally or alternatively be used. In a specific example, bacterial protein regions that match with human proteins are saved.

In embodiments, the method and/or system can include a second stage (and/or can be performed at any suitable time and frequency), which can include bacterial proteins regions obtained in the first stage (and/or at any suitable time and frequency) being analyzed to find HLA-class II epitopes. In specific examples, HLA-class II alleles are considered depending on each health condition or disease. In specific examples, this can be performed by one or more tools (e.g., Propred, IEDB, etc). In specific examples, proteins and/or peptide fragments that were predicted to have epitopes sequences can then be correlated with autoimmune diseases and/or conditions (e.g., by literature curation). In specific examples, cluster visualization can be performed to identify the predominant taxonomic order of those bacteria predicted to have proteins implied in a specific disease and/or condition.

In embodiments, the method and/or system can include a later stage (and/or can be performed at any suitable time and frequency), which can include generating peptide inhibitors targeting bacterial proteins. In examples, first, a structural model of the bacterial protein and/or epitope should be obtained from a structural database (e.g. Protein Data Bank PDB, etc.). In examples, if the sequence is short, peptide modelling can additionally or alternatively apply. In examples, if the protein fragment is large, a homology model can be built. The receptor, MHC-class II molecule, can be obtained from the structural database (e.g., PDB, etc.) and/or modelled according to the allele associated with the health condition under study (e.g., lupus risk alleles are HLA-DR3 and HLA-DR15).

Embodiments of the method and/or system can additionally or alternatively include:

One or more methodologies to identify bacterial proteins, peptides, and/or other suitable components responsible for triggering an autoimmune reaction.

One or more methodologies to obtain inhibitory peptides, proteins, and/or other suitable components against bacterial proteins, peptides, and/or other suitable components that mediate autoimmune reactions.

Embodiments can additionally or alternatively include applying any suitable approaches described herein for identification, generation, application, provision, and/or other suitable usage (e.g., in therapeutic compositions, etc.) of any suitable peptides, proteins, and/or other components, such as for any suitable autoimmune conditions (e.g., described herein, etc.).

### Specific Examples

As an example, an inhibitory lead peptide can be obtained from the bacterial protein binding region in the MHC-class II receptor. In examples, by using in-silico reengineering aided by molecular docking, that means, by producing single or double mutations in the lead peptide, a peptide with higher affinity to bacterial protein than the original MHC-class II binding site can be generated. Thus, in examples, it is expected that the new peptide can inhibit by competition the bacterial protein binding to MHC-class II receptor. In specific examples, some considerations can be taken into account, for example, the inhibitory peptides should not cross-react with human proteins triggering other autoimmune responses; to meet this requirement, inhibitory peptides can be searched against proteins/peptides found in the first stage (and/or at any suitable time and frequency), which are candidates to be autoimmune protein candidates.

In an example, embodiments can include and/or otherwise be applied for the targeting of Ro60 antigen orthologue bacterial protein. In humans, Ro60 protein has a RNA repair role (e.g., as shown in FIG. 2). However, in lupus disease patients, antibodies are generated against this antigen. Moreover, this antigen has orthologs in the microbiota (in Bacteroides thetaiotaomicron in gut), thus an increased immunity response (and excessive antibodies generation) is provoked. In that regard, Ro60 from bacteria can produce a chronic stimulus. In a specific example, one or more peptides, proteins, and/or other suitable components that prevent MHC-II binding to Ro60 bacterial protein can be designed, generated, provided, applied, and/or otherwise used (e.g., in a therapeutic composition, etc.).

However, any suitable portions, approaches, and/or steps described above and/or herein can be performed in any suitable sequence, and at any suitable time and frequency.

In embodiments, according to the mentioned antecedents, new pathogen-selective HtrA inhibitors might represent a new drug discovery opportunity. In embodiments, the method and/or system can include and/or otherwise prevent E-cadherin cleavage mediated by HtrA proteins from H.pylori. In embodiments, the method and/or system can include and/or otherwise identify and generate inhibitors of the proteolytic region of HrtA proteins from H.pylori, aimed to prevent E-cadherin binding and cleavage. In embodiments, the method and/or system can include, determine, provide, generate, administer, and/or otherwise facilitate new drugs, such as drugs that can be used to prevent attachment and/or cleavage mediated by H.pylori, thus they can be used as palliative and/or as a treatment against gastric cancer and/or any other suitable gastrointestinal conditions, cancers, and/or other suitable conditions.

The crystal structure of H. pylori HtrA with a deletion of the PDZ2 domain (PDB ID: 5Y28) with a resolution of 3.08 Å is obtainable, and additional characteristics regarding this structure can additionally or alternatively be determined. In variations, the method and/or system can include and/or be used to generate one or more trimeric homology model(s) including PDZ2 domain (sequence UNIPROT ID: G2J5T2), such as where DegS protein from E. coli can be used as a template (PDB: 4RQY) but any suitable proteins and/or or microorganisms can be used for templates. In a specific example, the homologous region between both proteins includes 37% sequence identity and 67% sequence similarity. In examples, the homology model and the crystal structure in PDB ID: 5Y28 can be structurally aligned and the PDZ1 and the proteolytic domain are structurally similar (RMSD). A potential allosteric site in each monomer of the trimer can act as a potential site (e.g., ideal site) for drug binding, which can facilitate preventing pathogen transmigration across the gastric epithelial barrier.

In examples, the method and/or system can include and/or be used to perform, after the HtrA homology model is built (and/or at any suitable time and frequency), the control binding affinity of an in-silico reported inhibitor was calculated as a reference through docking simulations. In a specific example, this binding energy was calculated in −7.5 kcal/mol. Compounds can exist (K_{D}=13 μM and IC₅₀=26 μM), which according to our docking calculation has HtrA binding energy of −7.7 kcal/mol and −8.1 kcal/mol. Embodiments can include, In the search of new possible inhibitors of HtrA proteolytic function, screening a set of molecules from a suitable source (e.g. CHEMBL database and/or any other suitable databases and/or other suitable sources) against HtrA enzyme using massive molecular docking simulations (and/or other suitable in silico approaches and/or other suitable approaches; etc.). In embodiments, the method and/or system can include applying any suitable set of criteria (e.g., thresholds; etc.). In examples, from this set, only molecules with a Tanimoto similarity coefficient higher than 0.5 compared with a reported inhibitor were considered; however, any suitable thresholds (e.g., any suitable Tanimoto similarity coefficient value; etc.) and/or other suitable criteria for the Tanimoto similarity coefficient, other similarity coefficients, and/or other suitable metrics can be used. In examples, then, these molecules were filtered by applying Lipinski rules of druggability (and/or any suitable criteria). In specific examples, the Lipinski rules of druggability can include any one or more of: molecular weight <500 daltons, number of H-bonds donor <5, number of H-bonds acceptor <10, number of N and O atoms <15, range of partition coefficient log P between −2 and 5, number of rotatable bonds <10, number of ring number <10; and/or any other suitable criteria.

The present application also relates to a method of treating a bacterial infection comprising administering a pharmaceutically effective amount of the HtrA inhibitor described herein to a human subject in need thereof. In an embodiment, the bacterial infection is a Helicobacter pylori infection.

The following includes a specific example of a main scaffold (Formula (I)):

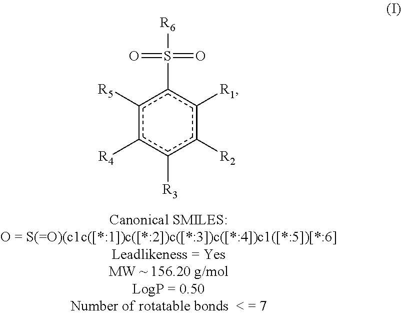

wherein R₁ to R₆ are defined below and the dotted line indicates the bond is either none, a single bond or a double bond. For clarity, each cell of Tables 1-6 illustrates the chemical structure of the substituent on the top and its Canonical SMILES at the bottom. “A” indicates either H or the connection position of the group. For example, “A-Cl” indicates that the substituent is —Cl; “A-” indicates that the substituent is —CH₃. If there are two or more “A” in the chemical structure, each A is independently either H or the connection position.

### R₁

R₁ can be H, halo, cyano, OH, (C₁-C₆)alkyl, (C₃-C₁₀)heterocyclo, (C₃-C₁₀)cycloalkyl, aryl, or heteroaryl, any of which may be optionally substituted with 1 or more R^{w} groups as allowed by valence; wherein R^{w} at each occurrence is independently H, halo, cyano, nitro, oxo, amino, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl, wherein n is 0, 1, 2, 3, 4, 5, or 6.

In some embodiments, R₁ is selected from the groups listed in Table 1.

TABLE 1
R₁ substituent
A—Cl
Cl*
A
[*H]

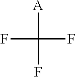

FC(F)(F)*

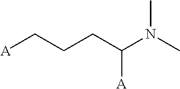

C(CC(N(C)C)*)C*
A—F
F*

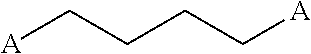

C(CC*)C*
A—
C*

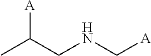

C(NC*)C(C)*

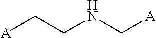

C(NC*)C*

### R₂

R₂ can be H, halo, cyano, OH, (C₁-C₆)alkyl, (C₂-C₈)alkenyl, (C₁-C₆)alkoxy, (C₃-C₁₀)heterocyclo, (C₃-C₁₀)cycloalkyl, aryl, or heteroaryl, any of which may be optionally substituted with 1 or more R^{w} groups as allowed by valence; wherein R^{w} at each occurrence is independently H, halo, cyano, nitro, oxo, amino, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl, wherein n is 0, 1, 2, 3, 4, 5, or 6.

In some embodiments, R₂ is selected from the groups listed in Table 2.

TABLE 2
R₂ substitutent
A—Cl
C*

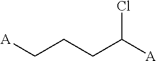

C(CC(Cl)*)C*

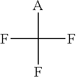

FC(F)(F)*

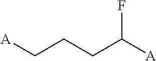

C(CC(F)*)C*
A—F
F*

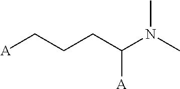

C(CC(N(C)C)*)C*

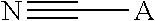

N#C*

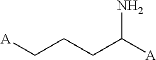

C(CC(N)*)C*

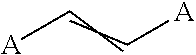

C(═C*)C*

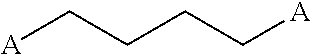

C(CC*)C*

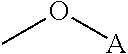

CO*

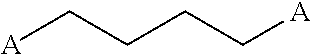

C(CC*)C*
A—
C*

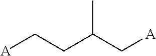

C(C(C*)C)C*

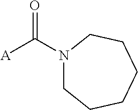

ClN(C(═O)*)CCCCCl

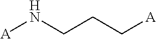

C(N*)CC*
A
[*H]

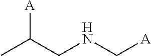

C(NC*)C(C)*

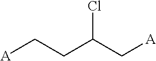

C(C(Cl)C*)C*

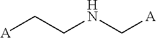

C(NC*)C*

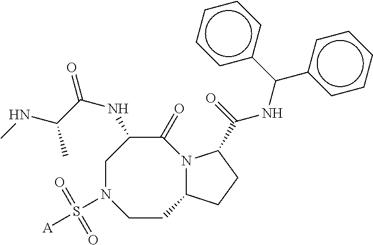

c1ccc(C(c2ccccc2)NC(═O)[C@H]2N3C(═O)[C@@H](NC(═O)
[C@@H](NC)C)CN(S(═O)(═O)*)CC[C@H]3CC2)cc1

### R₃

R₃ can be H, halo, cyano, OH, (C₁-C₆)alkyl, (C₂-C₈)alkenyl, (C₂-C₈)carboxyalkyl; N—(C₁-C₆)alkylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more Rw groups as allowed by valence; wherein Rw at each occurrence is independently HK halo, cyano, nitro, oxo, amino, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C3-C10)heterocyclo, (C3-C10)cycloalkyl, —(CH2)n-(C3-C10) cycloalkyl, —(CH2)n-(C3-C10)heterocyclo, —(CH2)n-aryl, —(CH2)n-heteroaryl, aryl, and heteroaryl, wherein n is 0, 1, 2, 3, 4, 5, or 6.

In some embodiments, R₃ is selected from the groups listed in Table 3.

TABLE 3
R₃ substituent
A—Br
Br*

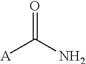

NC(═O)*

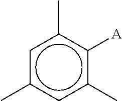

c1c(C)cc(C)c(*)c1C
A—Cl
Cl*
A—NH₂
N*

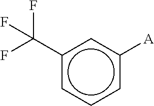

c1cc(C(F)(F)F)cc(*)c1

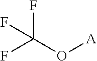

FC(F)(F)O*

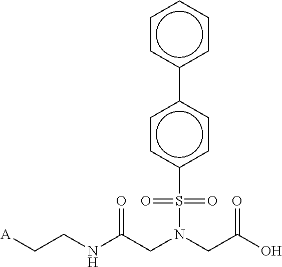

OC(═O)CN(CC(═O)NCC*)S(═O)(═O)c1ccc(c2ccccc2)cc1

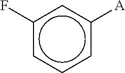

c1cc(F)cc(*)c1

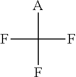

FC(F)(F)*
A—OH
O*

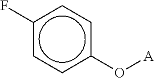

c1cc(O*)ccc1F
A—F
F*

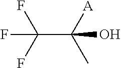

O[C@@](C)(C(F)(F)F)*

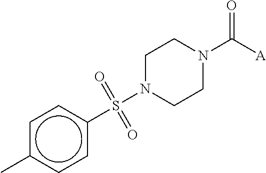

c1cc(S(═O)(═O)N2CCN(C(═O)*)CC2)ccc1C

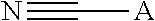

N#C*

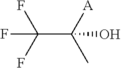

O[C@](C)(C(F)(F)F)*

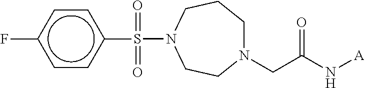

c1cc(S(═O)(═O)N2CCN(CC(═O)N*)CCC2)ccc1F

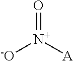

O═[N+]([O−])*

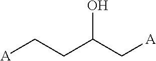

OC(CC*)C*

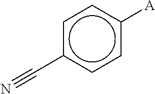

c1cc(*)ccc1C#N

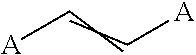

C(═C*)C*
A
[*H]

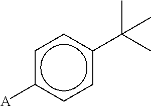

c1cc(*)ccc1C(C)(C)C

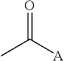

CC(═O)*

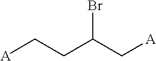

C(C(Br)C*)C*

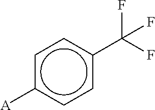

c1cc(*)ccc1C(F)(F)F

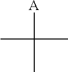

CC(C)(C)*

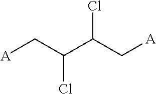

C(C(Cl)C(Cl)C*)*

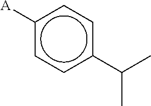

c1cc(*)ccc1C(C)C

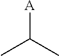

CC(C)*

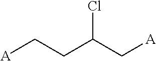

C(C(Cl)C*)C*

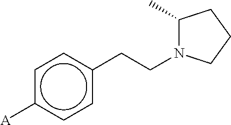

c1cc(*)ccc1CCN1CCC[C@H]1C

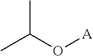

CC(O*)C

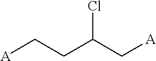

C(C(Cl)C*)C*

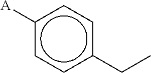

c1cc(*)ccc1CC

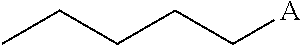

CCCCC*

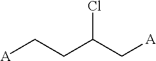

C(C(Cl)C*)C*

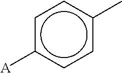

c1cc(*)ccc1C

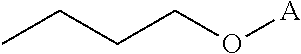

CCCCO*

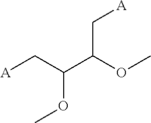

C(C(OC)C(OC)C*)*

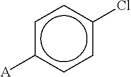

c1cc(*)ccc1Cl

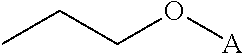

CCCO*

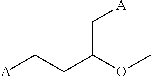

C(C(OC)C*)C*

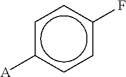

c1cc(*)ccc1F

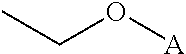

CCO*

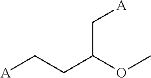

C(C(OC)C*)C*

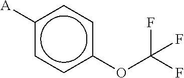

c1cc(*)ccc1OC(F)(F)F

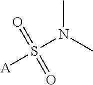

CN(C)S(═O)(═O)*

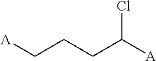

C(CC(Cl)*)C*

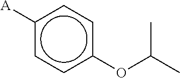

c1cc(*)ccc1OC(C)C

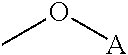

CO*

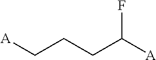

C(CC(F)*)C*

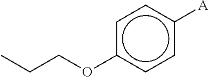

c1cc(*)ccc1OCCC

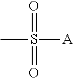

CS(═O)(═O)*

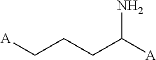

C(CC(N)*)C*

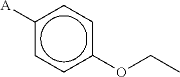

c1cc(*)ccc1OCC
A—
C*

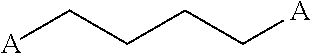

C(CC*)C*

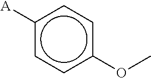

c1cc(*)ccc1OC

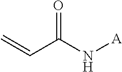

C═CC(═O)N*

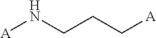

C(N*)CC*

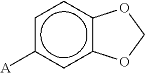

c1cc(*)cc2c1OCO2

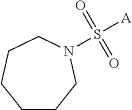

C1N(S(═O)(═O)*)CCCCC1

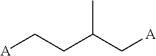

C(C(C*)C)C*

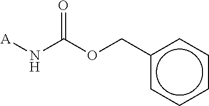

c1ccc(COC(═O)N*)cc1

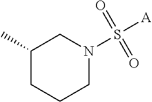

C1N(S(═O)(═O)*)C[C@@H](C)CC1

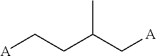

C(C(C*)C)C*

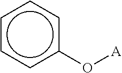

c1ccc(O*)cc1

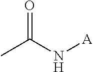

N(C(═O)C)*

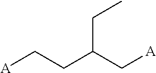

C(C(C*)CC)C*

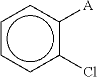

c1ccc(*)c(Cl)c1

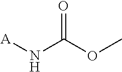

N(C(═O)OC)*

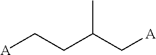

C(C(C*)C)C*

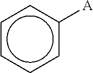

c1ccc(*)cc1

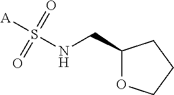

N(C[C@@H]1OCCC1)S(═O)(═O)*

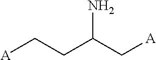

C(C(C*)N)C*

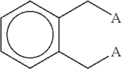

c1ccc(C*)c(C*)c1

### R₄

R₄ can be H, halo, cyano, OH, (C₁-C₆)alkyl, (C₂-C₈)akenyl, (C₂-C₈)carboxyalkyl; N—(C₁-C₆)akylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence; wherein R^{w} at each occurrence is independently H, halo, cyano, nitro, oxo, amino, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl, wherein n is 0, 1, 2, 3, 4, 5, or 6.

In some embodiments, R₄ is selected from the groups listed in Table 4.

TABLE 4
R₄ substituent
A—Cl
Cl*

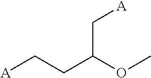

C(C(OC)C*)C*

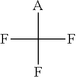

FC(F)(F)*

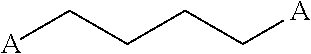

C(CC*)C*
A—F
F*

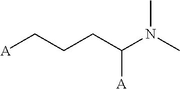

C(CC*)C(N(C)C)*
A—
C*

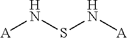

N(SN*)*
A—OH
O*

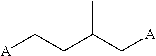

C(C(C*)C)C*

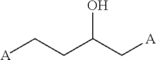

OC(CC*)C*

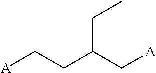

C(C(C*)CC)C*
A
[*H]

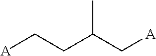

C(C(C*)C)C*

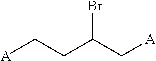

C(C(Br)C*)C*

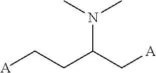

C(C(C*)N(C)C)C*

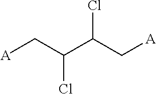

C(C(Cl)C(Cl)C*)*

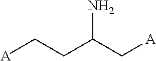

C(C(C*)N)C*

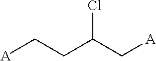

C(C(Cl)C*)C*

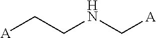

C(NC*)C*

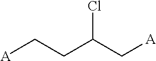

C(C(Cl)C*)C*

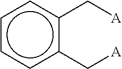

c1ccc(C*)c(C*)c1

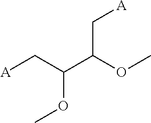

C(C(OC)C(OC)C*)*

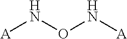

N(ON*)*

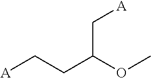

C(C(OC)C*)C*

### R₅

R₅ can be H, halo, cyano, OH, (C₁-C₆)alkyl, (C₂-C₈)akenyl, (C₂-C₈)carboxyalkyl; N—(C₁-C₆*akylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence; wherein R^{w} at each occurrence is independently H, halo, cyano, nitro, oxo, amino, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl, wherein n is 0, 1, 2, 3, 4, 5, or 6.

In some embodiments R₅ is selected from the groups listed in Table 5.

TABLE 5
R₅ substituent
A—Br
Br*

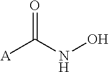

ONC(═O)*
A—Cl
Cl*
A
[*H]

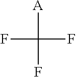

FC(F)(F)*

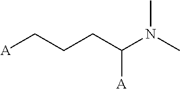

C(CC*)C(N(C)C)*
A—F
F*

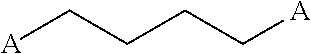

C(CC*)C*
A—I
I*

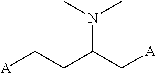

C(C(C*)N(C)C)C*

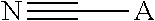

N#C*

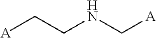

C(NC*)C*

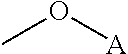

CO*

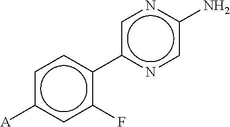

c1nc(N)cnc1c1ccc(*)cc1F
A—
C*

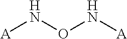

N(ON*)*

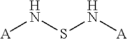

N(SN*)*

### R₆

R₆ can be H, halo, cyano, OH, (C₁-C₆)alkyl, (C₂-C₈)alkenyl, (C₂-C₈)carboxyalkyl; N—(C₁-C₆)akylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence; wherein R^{w} at each occurrence is independently H, halo, cyano, nitro, oxo, amino, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl, wherein n is 0, 1, 2, 3, 4, 5, or 6.

In some embodiments, R₆ is selected from the groups listed in Table 6.

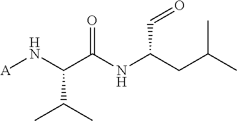

C(═O)[C@@H](NC(═O)[C@@H](N*)C(C)C)CC(C)C

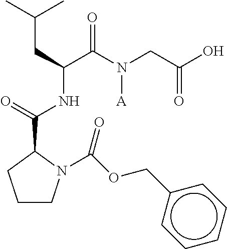

OC(═O)CN(C(═O)[C@@H](NC(═O)
[C@H]1N(C(═O)OCc2ccccc2)CCC1)CC(C)C)*

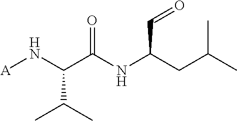

C(═O)[C@H](NC(═O)[C@@H](N*)C(C)C)CC(C)C

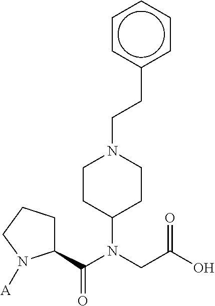

OC(═O)CN(C(═O)[C@H]1N(*)CCC1)C1CCN(CCc2ccccc2)CC1

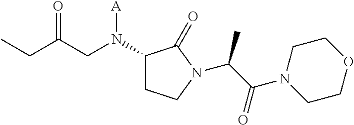

C(C(═O)CC)N([C@@H]1C(═O)N([C@H](C(═O)N2CCOCC2)C)CC1)*

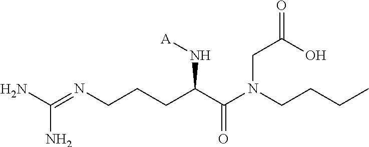

OC(═O)CN(C(═O)[C@H](N*)CCCN═C(N)N)CCCC

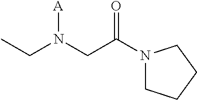

C(C(═O)N1CCCC1)N(CC)*

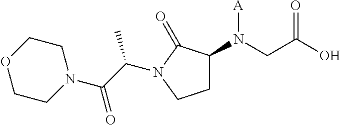

OC(═O)CN([C@@H]1C(═O)N([C@H](C(═O)N2CCOCC2)C)CC1)*

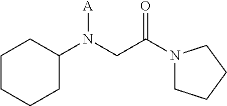

C(C(═O)N1CCCC1)N(C1CCCCC1)*

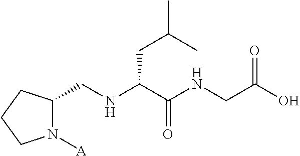

OC(═O)CNC(═O)[C@H](NC[C@@H]1N(*)CCC1)CC(C)C

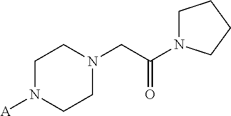

C(C(═O)N1CCCC1)N1CCN(*)CC1

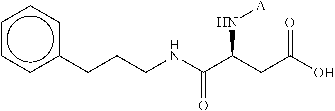

OC(═O)C[C@@H](C(═O)NCCCc1ccccc1)N*

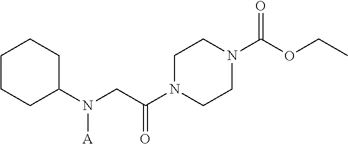

C(C(═O)N1CCN(C(═O)OCC)CC1)N(C1CCCCC1)*

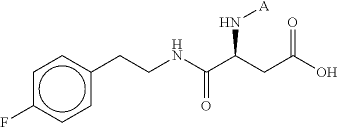

OC(═O)C[C@@H](C(═O)NCCc1ccc(F)cc1)N*

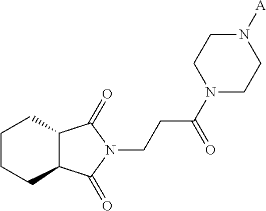

C(C(═O)N1CCN(*)CC1)CN1C(═O)[C@H]2CCCC[C@@H]2C1═O

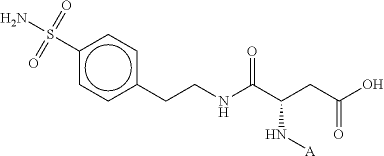

OC(═O)C[C@@H](C(═O)NCCc1ccc(S(═O)(═O)N)cc1)N*

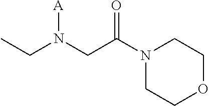

C(C(═O)N1CCOCC1)N(CC)*

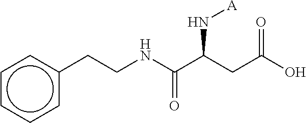

OC(═O)C[C@@H](C(═O)NCCc1ccccc1)N*

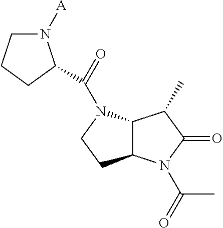

CC(═O)N1C(═O)[C@@H](C)[C@H]2N(C(═O)[C@H]3N(*)CCC3)CC[C@H]12

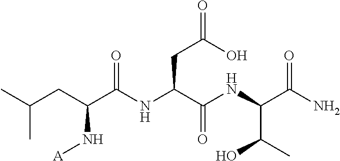

OC(═O)C[C@@H](C(═O)N[C@@H](C(═O)N)[C@H](O)C)NC(═O)[C@@H](N*)CC(C)C

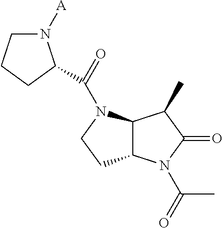

CC(═O)N1C(═O)[C@H](C)[C@@H]2N(C(═O)[C@H]3N(*)CCC3)CC[C@@H]12

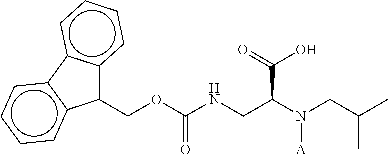

OC(═O)[C@@H](N(CC(C)C)*)CNC(═O)OCC1c2ccccc2c2ccccc12

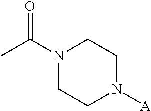

CC(═O)N1CCN(*)CC1

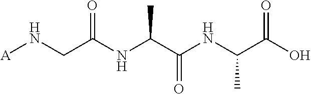

OC(═O)[C@@H](NC(═O)[C@@H](NC(═O)CN*)C)C

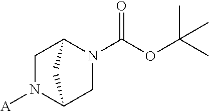

CC(OC(═O)N1C[C@H]2N(*)C[C@@H]1C2)(C)C

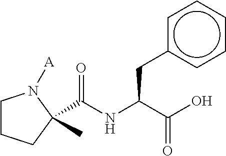

OC(═O)[C@@H](NC(═O)[C@@]1(C)N(*)CCC1)Cc1ccccc1

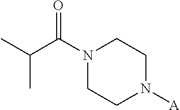

CC(C(═O)N1CCN(*)CC1)C

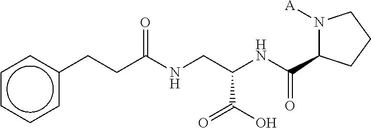

OC(═O)[C@@H](NC(═O)[C@H]1N(*)CCC1)CNC(═O)CCc1ccccc1

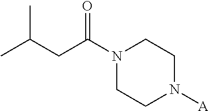

CC(C)CC(═O)N1CCN(*)CC1

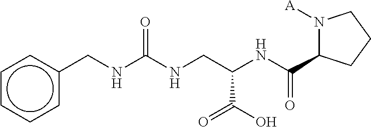

OC(═O)[C@@H](NC(═O)[C@H]1N(*)CCC1)CNC(═O)NCc1ccccc1

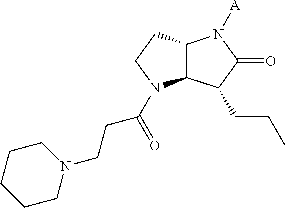

CCC[C@H]1C(═O)N(*)[C@H]2CCN(C(═O)CCN3CCCCC3)[C@H]12

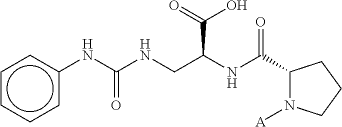

OC(═O)[C@@H](NC(═O)[C@H]1N(*)CCC1)CNC(═O)Nc1ccccc1

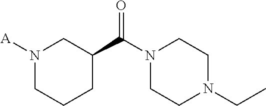

CCN1CCN(C(═O)[C@@H]2CN(*)CCC2)CC1

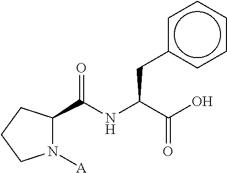

OC(═O)[C@@H](NC(═O)[C@H]1N(*)CCC1)Cc1ccccc1

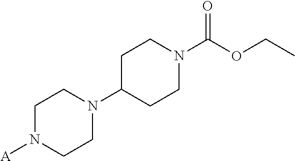

CCOC(═O)N1CCC(N2CCN(*)CC2)CC1

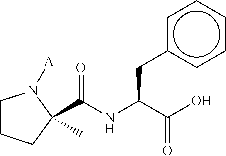

OC(═O)[C@@H](NC(═O)[C@]1(C)N(*)CCC1)Cc1ccccc1

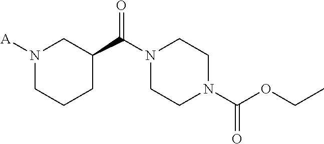

CCOC(═O)N1CCN(C(═O)[C@@H]2CN(*)CCC2)CC1

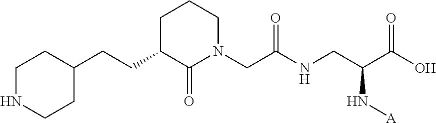

OC(═O)[C@@H](N*)CNC(═O)CN1C(═O)[C@H](CCC2CCNCC2)CCC1

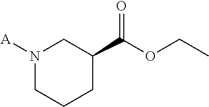

CCOC(═O)[C@@H]1CN(*)CCC1

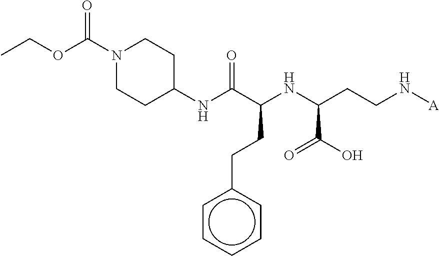

OC(═O)[C@@H](N[C@H](C(═O)NC1CCN(C(═O)OCC)CC1)CCc1ccccc1)CCN*

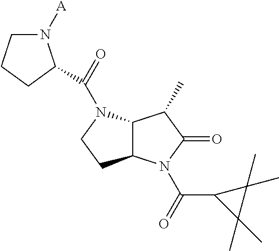

CC1(C)C(C(═O)N2C(═O)[C@@H](C)[C@H]3N(C(═O)[C@H]4N(*)CCC4)
CC[C@H]23)C1(C)C

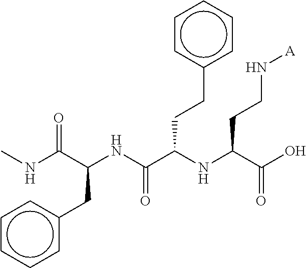

OC(═O)[C@@H](N[C@H](C(═O)N[C@H](C(═O)NC)Cc1ccccc1)CCc1ccccc1)CCN*

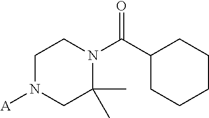

CC1(C)N(C(═O)C2CCCCC2)CCN(*)C1

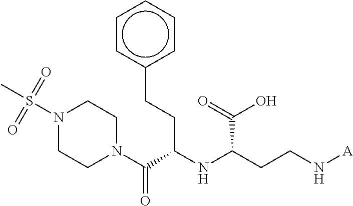

OC(═O)[C@@H](N[C@H](C(═O)N1CCN(S(═O)(═O)C)CC1)CCc1ccccc1)CCN*

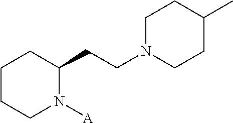

CC1CCN(CC[C@H]2N(*)CCCC2)CC1

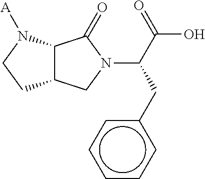

OC(═O)[C@@H](N1C(═O)[C@H]2N(*)CC[C@H]2C1)Cc1ccccc1

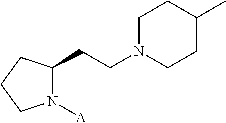

CC1CCN(CC[C@H]2N(*)CCC2)CC1

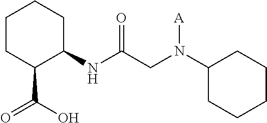

OC(═O)[C@@H]1CCCC[C@H]1NC(═O)CN(C1CCCCC1)*

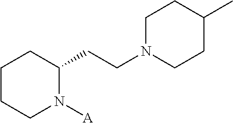

CC1CCN(CC[C@@H]2N(*)CCCC2)CC1

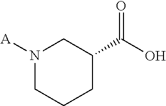

OC(═O)[C@H]1CN(*)CCC1

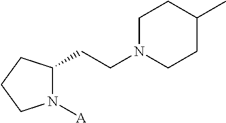

CC1CCN(CC[C@@H]2N(*)CCC2)CC1

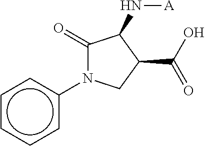

OC(═O)[C@H]1CN(c2ccccc2)C(═O)[C@H]1N*

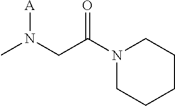

CN(CC(═O)N1CCCCC1)*

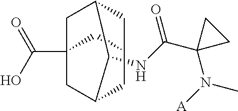

OC(═O)[C@@]12C[C@H]3C[C@@H](C1)[C@H](NC(═O)C1(N(C)*)CC1[C@H](C3)C2

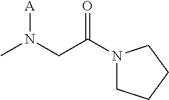

CN(CC(═O)N1CCCC1)*

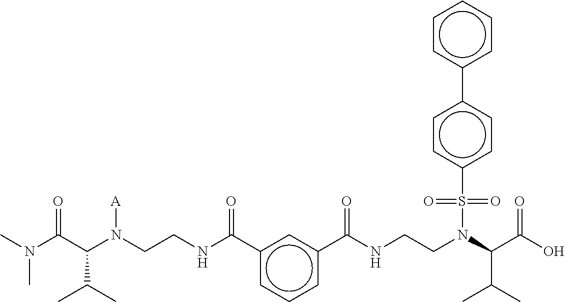

OC(═O)[C@H](N(CCNC(═O)c1cccc(C(═O)NCCN([C@@H](C(═O)N(C)C)C(C)C)*)c1)
S(═O)(═O)c1ccc(c2ccccc2)cc1)C(C)C

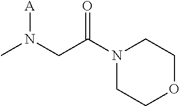

CN(CC(═O)N1CCOCC1)*

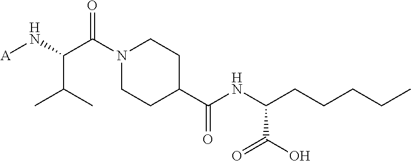

OC(═O)[C@H](NC(═O)C1CCN(C(═O)[C@@H](N*)C(C)C)CC1)CCCCC

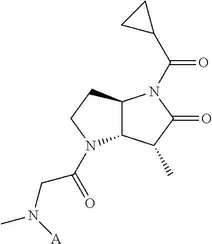

CN(CC(═O)N1CC[C@H]2N(C(═O)C3CC3)C(═O)[C@H](C)[C@H]12)*

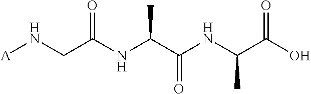

OC(═O)[C@H](NC(═O)[C@@H](NC(═O)CN*)C)C

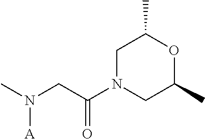

CN(CC(═O)N1C[C@H](C)O[C@@H](C)C1)*

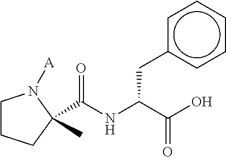

OC(═O)[C@H](NC(═O)[C@@]1(C)N(*)CCC1)Cc1ccccc1

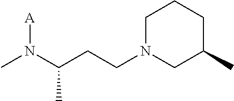

CN([C@@H](C)CCN1CCC[C@@H](C)C1)*

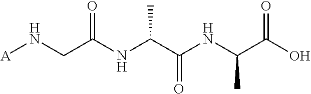

OC(═O)[C@H](NC(═O)[C@H](NC(═O)CN*)C)C

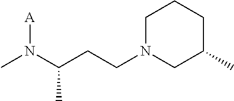

CN([C@@H](C)CCN1CCC[C@H](C)C1)*

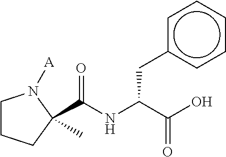

OC(═O)[C@H](NC(═O)[C@]1(C)N(*)CCC1)Cc1ccccc1

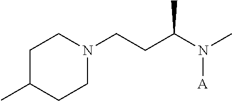

CN([C@H](C)CCN1CCC(C)CC1)*

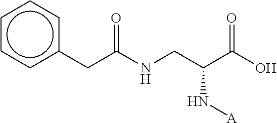

OC(═O)[C@H](N*)CNC(═O)Cc1ccccc1

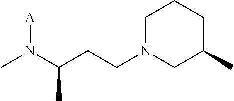

CN([C@H](C)CCN1CCC[C@@H](C)C1)*

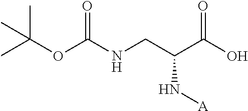

OC(═O)[C@H](N*)CNC(═O)OC(C)(C)C

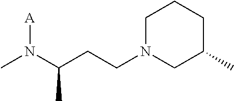

CN([C@H](C)CCN1CCC[C@H](C)C1)*

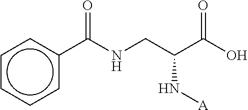

OC(═O)[C@H](N*)CNC(═O)c1ccccc1

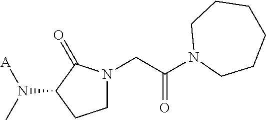

CN([C@@H]1C(═O)N(CC(═O)N2CCCCCC2)CC1)*

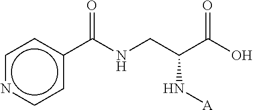

OC(═O)[C@H](N*)CNC(═O)c1ccncc1

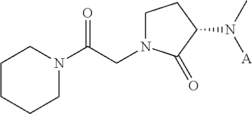

CN([C@@H]1C(═O)N(CC(═O)N2CCCCC2)CC1)*

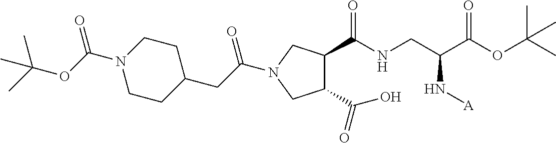

OC(═O)[C@@H]1CN(C(═O)CC2CCN(C(═O)OC(C)(C)C)CC2)C[C@H]1C
(═O)NC[C@@H](C(═O)OC(C)(C)C)N*

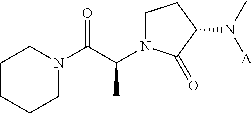

CN([C@@H]1C(═O)N([C@H](C(═O)N2CCCCC2)CC1)*

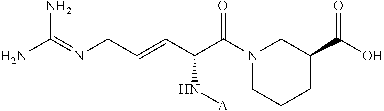

OC(═O)[C@@H]1CN(C(═O)[C@@H](/C═C/CN═C(N)N)N*)CCC1

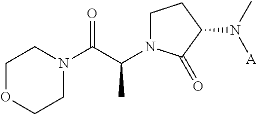

CN([C@@H]1C(═O)N([C@H](C(═O)N2CCOCC2)C)CC1)*

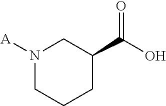

OC(═O)[C@@H]1CN(*)CCC1

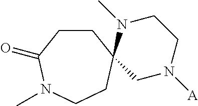

CN1C(═O)CC[C@@]2(N(C)CCN(*)C2)CC1

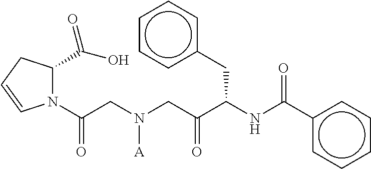

OC(═O)[C@@H]1N(C(═O)CN(CC(═O)[C@@H](NC(═O)c2ccccc2)Cc2ccccc2)*)C═CC1

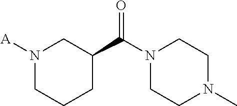

CN1CCN(C(═O)[C@@H]2CN(*)CCC2)CC1

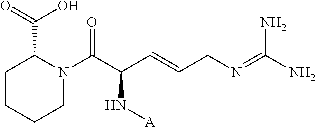

OC(═O)[C@@H]1N(C(═O)[C@@H](/C═C/CN═C(N)N)N*)CCCC1

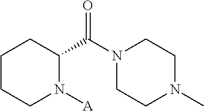

CN1CCN(C(═O)[C@@H]2N(*)CCCC2)CC1

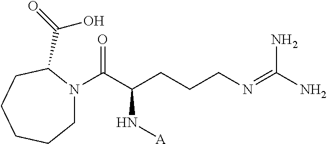

OC(═O)[C@@H]1N(C(═O)[C@H](N*)(CCCN═C(N)N)CCCCC1

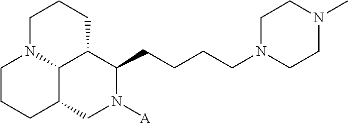

CN1CCN(CCCC[C@H]2CN(*)C[C@@H]3CCCN4CCC[C@H]2[C@H]34)CC1

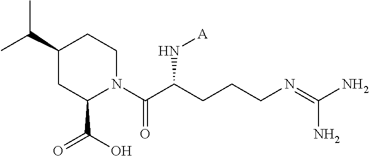

OC(═O)[C@@H]1N(C(═O)[C@H](N*)CCCN═C(N)N)CC[C@H](C(C)C)C1

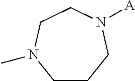

CN1CCN(*)CCC1

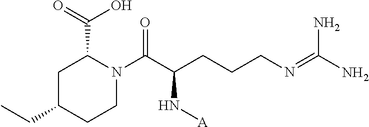

OC(═O)[C@@H]1N(C(═O)[C@H](N*)CCCN═C(N)N)CC[C@H](CC)C1

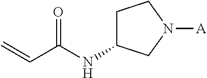

C═CC(═O)N[C@H]1CN(*)CC1

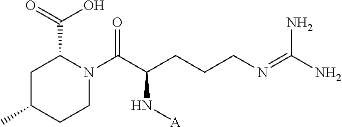

OC(═O)[C@@H]1N(C(═O)[C@H](N*)CCCN═C(N)N)CC[C@H](C)C1

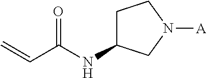

C═CC(═O)N[C@@H]1CN(*)CC1

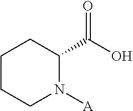

OC(═O)[C@@H]1N(*)CCCC1

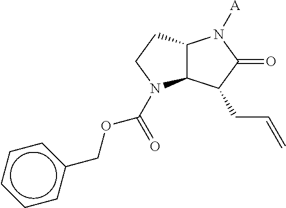

C═CC[C@H]1C(═O)N(*)[C@H]2CCN(C(═O)OCc3ccccc3)[C@H]12

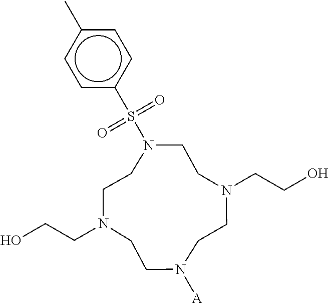

OCCN1CCN(S(═O)(═O)c2ccc(C)cc2)CCN(CCO)CCN(*)CC1

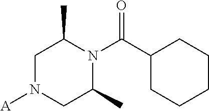

C1CCC(C(═O)N2[C@@H](C)CN(*)C[C@H]2C)CC1

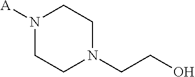

OCCN1CCN(*)CC1

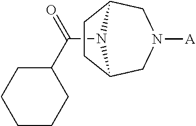

C1CCC(C(═O)N2[C@@H]3CN(*)C[C@H]2CC3)CC1

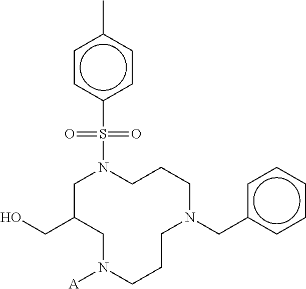

OCCN1CCN(S(═O)(═O)c2ccc(C)cc2)CCCN(Cc2ccccc2)CCCN(*)C1

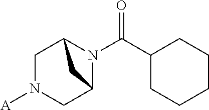

C1CCC(C(═O)N2[C@@H]3CN(*)C[C@H]2C3)CC1

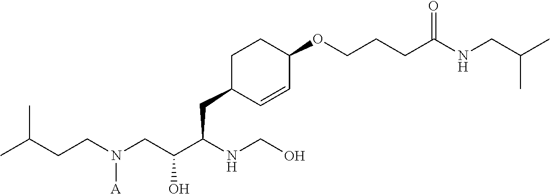

OCN[C@H](C[C@@H]1C═C[C@H](OCCCC(═O)NCC(C)C)CC1)[C@H](O)CN(CCC(C)C)*

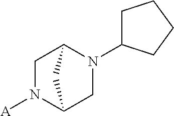

C1CCC(N2C[C@H]3N(*)C[C@@H]2C3)C1

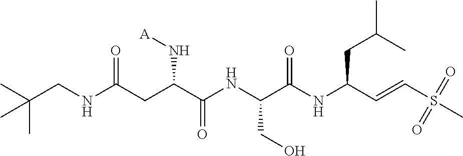

OC[C@@H](C(═O)N[C@H](/C═C/S(═O)(═O)CC(C)C)NC(═O)[C@@H]
(*N)CC(═O)NCC(C)(C)C

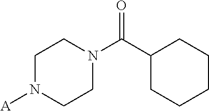

C1N(C(═O)C2CCCCC2)CCN(*)C1

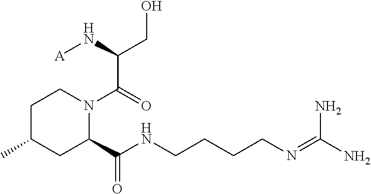

OC[C@@H](C(═O)N1CC[C@@H](C)C[C@@H]1C(═O)NCCCCN═C(N)N)N*

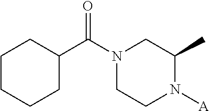

C1N(C(═O)C2CCCCC2)C[C@@H](C)N(*)C1

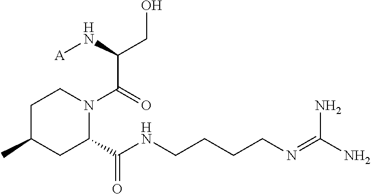

OC[C@@H](C(═O)N1CC[C@H](C)C[C@H]1C(═O)NCCCCN═C(N)N)N*

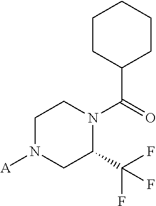

C1N(C(═O)C2CCCCC2)[C@H](C(F)(F)F)CN(*)C1

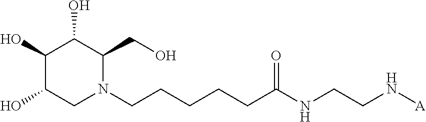

OC[C@H]1N(CCCCCC(═O)NCCN*)C[C@H](O)[C@@H](O)[C@@H]1O

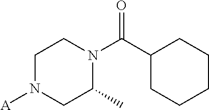

C1N(C(═O)C2CCCCC2)[C@H](C)CN(*)C1

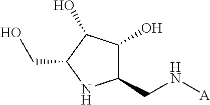

OC[C@H]1N[C@H](CN*)[C@@H](O)[C@H]1O

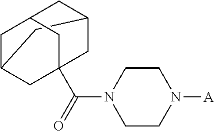

C1N(C(═O)C23CC4CC(CC(C4)C2)C3)CCN(*)C1

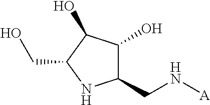

OC[C@H]1N[C@H](CN*)[C@@H](O)[C@@H]1O

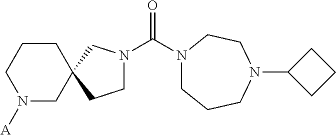

C1N(C(═O)N2CC[C@@]3(C2)CN(*)CCC3)CCCN(C2CCC2)C1

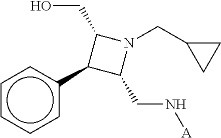

OC[C@@H]1N(CC2CC2)[C@H](CN*)[C@H]1c1ccccc1

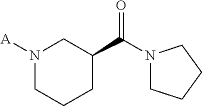

C1N(C(═O)[C@@H]2CN(*)CCC2)CCC1

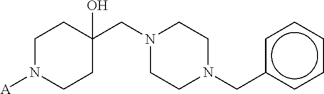

OC1(CN2CCN(Cc3ccccc3)CC2)CCN(*)CC1

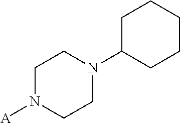

C1N(C2CCCCC2)CCN(*)C1

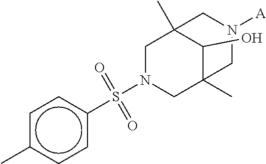

OC1C2(C)(CN(S(═O)(═O)c3ccc(C)cc3)CC1(C)CN(*)C2

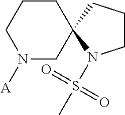

C1N(S(═O)(═O)C)[C@]2(CN(*)CCC2)CC1

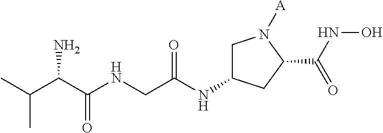

ONC(═O)[C@H]1N(*)C[C@@H](NC(═O)CNC(═O)[C@@H](N)C(C)C)C1

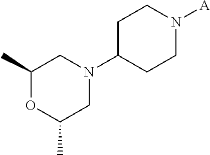

C1N(*)CCC(N2C[C@H](C)O[C@@H](C)C2)C1

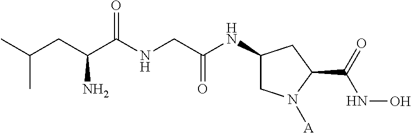

ONC(═O)[C@H]1N(*)C[C@@H](NC(═O)CNC(═O)[C@@H](N)CC(C)C)C1

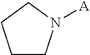

C1N(*)CCC1

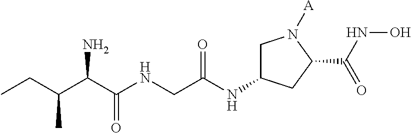

ONC(═O)[C@H]1N(*)C[C@@H](NC(═O)CNC(═O)[C@H](N)[C@@H](C)CC)C1

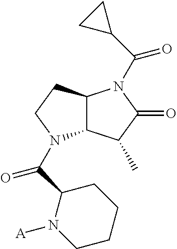

C1N(*)[C@@H](C(═O)N2CC[C@H]3N(C(═O)C4CC4)C(═O)[C@H](C)[C@H]23)CCC1

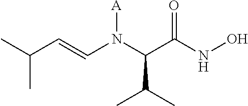

ONC(═O)[C@H](N(/C═C/C(C)C)*)C(C)C

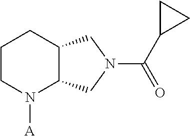

C1N(*)[C@H]2CN(C(═O)C3CC3)C[C@H]2CC1

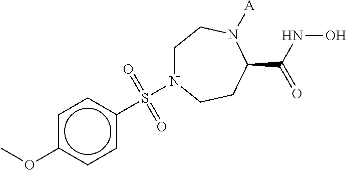

ONC(═O)[C@@H]1N(*)CCN(S(═O)(═O)c2ccc(OC)cc2)CC1

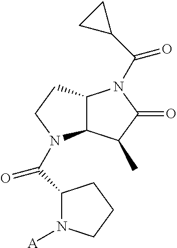

C1N(*)[C@H](C(═O)N2CC[C@@H]3N(C(═O)C4CC4)C(═O)[C@@H](C)[C@@H]23)CC1

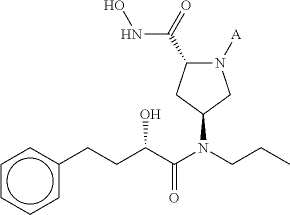

ONC(═O)[C@@H]1N(*)C[C@@H](N(C(═O)[C@@H](O)CCc2ccccc2)CCC)C1

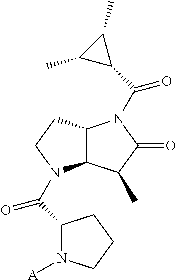

C1N(*)[C@H](C(═O)N2CC[C@@H]3N(C(═O)[C@H]4[C@@H](C)[C@H]4C)C(═O)
[C@@H](C)[C@@H]23)CC1

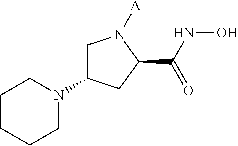

ONC(═O)[C@@H]1N(*)C[C@@H](N2CCCCC2)C1

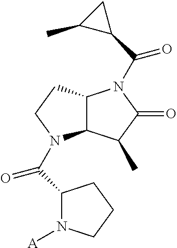

C1N(*)[C@H](C(═O)N2CC[C@@H]3N(C(═O)[C@@H]4C[C@@H]4C)C(═O)
[C@@H](C)[C@@H]23)CC1

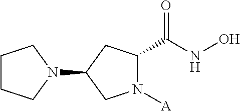

ONC(═O)[C@@H]1N(*)C[C@@H](N2CCCC2)C1

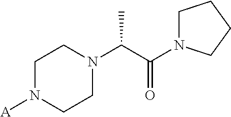

C1N([C@@H](C(═O)N2CCCC2)C)CCN(*)C1

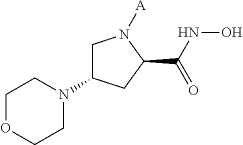

ONC(═O)[C@@H]1N(*)C[C@@H](N2CCOCC2)C1

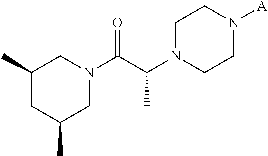

C1N([C@@H](C(═O)N2C[C@@H](C)C[C@@H](C)C2)C)CCN(*)C1

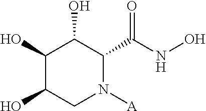

ONC(═O)[C@@H]1N(*)C[C@@H](O)[C@@H](O)[C@@H]1O

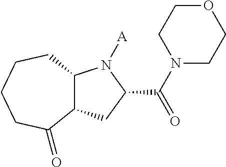

C1OCCN(C(═O)[C@H]2N(*)[C@H]3CCCCC(═O)[C@H]3C2)C1

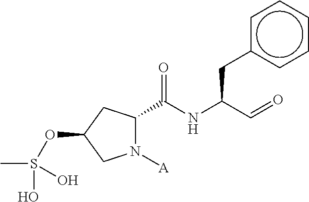

OS(O)(O[C@@H]1CN(*)[C@@H](C(═O)N[C@H](C═O)Cc2ccccc2)C1)C

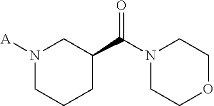

C1OCCN(C(═O)[C@@H]2CN(*)CCC2)C1

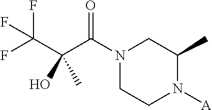

O[C@@](C(═O)N1CCN(*)[C@H](C)C1)(C)C(F)(F)F

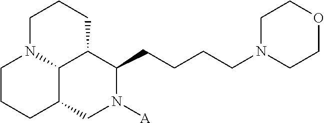

C1OCCN(CCCC[C@H]2N(*)C[C@@H]3CCCN4CCC[C@H]2[C@H]34)C1

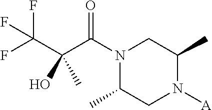

O[C@@](C(═O)N1C[C@@H](C)N(*)C[C@@H]1C)(C)C(F)(F)F

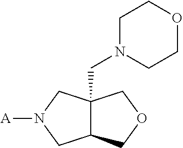

C1OCCN(C[C@]23COC[C@H]2CN(*)C3)C1

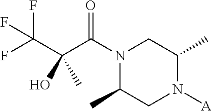

O[C@@](C(═O)N1C[C@H](C)N(*)C[C@H]1C)(C)C(F)(F)F

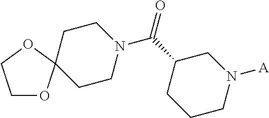

C1OC2(OC1)CCN(C(═O)[C@@H]1CN(*)CCC1)CC2

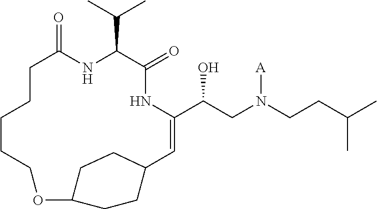

O[C@@H](/C/1═C/C2CCC(OCCCCCC(═O)N[C@@H](C(C)C)C(═O)N1)CC2)
CN(CCC(C)C)*

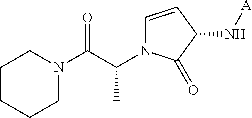

C1═C[C@H](N*)C(═O)N1[C@@H](C(═O)N1CCCCC1)C

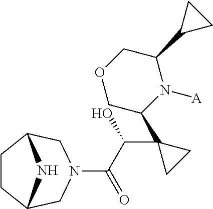

O[C@@H](C(═O)N1C[C@H]2N[C@@H](C1)(CC2)C1([C@@H]2N(*)[C@H]
(C3CC3)COC2)CC1

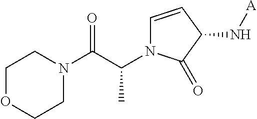

C1═C[C@H](N*)C(═O)N1[C@@H](C(═O)N1CCCCC1)C

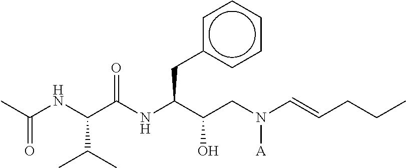

O[C@@H](CN(/C═C/CCC)*)[C@@H](NC(═O)[C@@H](NC(═O)C)C(C)C)Cc1ccccc1

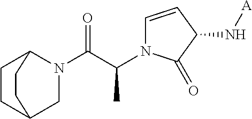

C1═C[C@H](N*)C(═O)N1[C@H](C(═O)N1CC2CCC1CC2)C

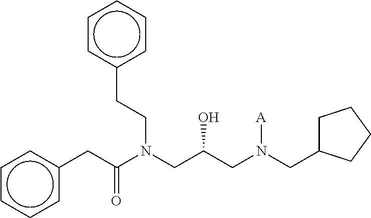

O[C@@H](CN(C(═O)Cc1ccccc1)CCc1ccccc1)CN(CC1CCCC1)*

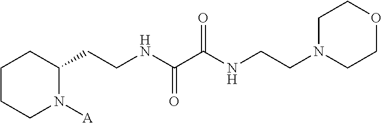

N(C(—O)C(—O)NCC[C@@H]1N(*)CCCC1)CCN1CCOCC1

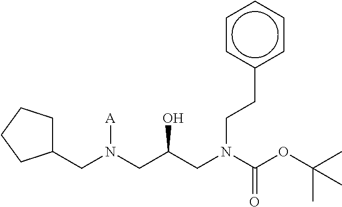

O[C@@H](CN(C(═O)OC(C)(C)C)CCc1ccccc1)CN(CC1CCCC1)*

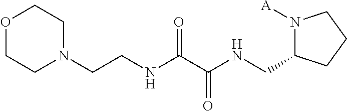

N(C(═O)C(═O)NC[C@@H]1N(*)CCC1)CCN1CCOCC1

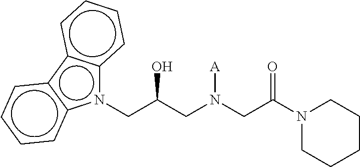

O[C@@H](CN(CC(═O)N1CCCCC1)*)Cn1c2ccccc2c2ccccc12

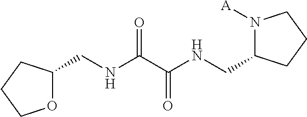

N(C(═O)C(═O)NC[C@@H]1N(*)CCC1)C[C@@H]1OCCC1

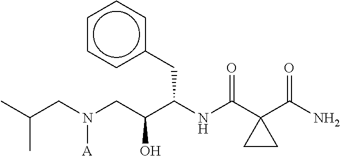

O[C@@H](CN(CC(C)C)*)[C@@H](NC(═O)C1(C(═O)N)CC1)Cc1ccccc1

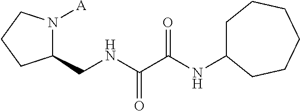

N(C(—O)C(—O)NC[C@@H]1N(*)CCC1)C1CCCCCC1

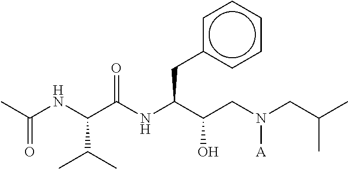

O[C@@H](CN(CC(C)C)*)[C@@H](NC(═O)[C@@H](NC(═O)C)C(C)C)Cc1ccccc1

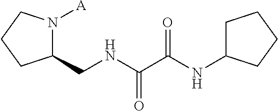

N(C(═O)C(═O)NC[C@@H]1N(*)CCC1)C1CCCC1

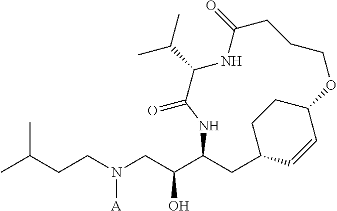

O[C@@H](CN(CCC(C)C)*)[C@H]1NC(═O)[C@H](C(C)C)NC(═O)CCCO[C@@H]
2C═C[C@@H](CC2)C1

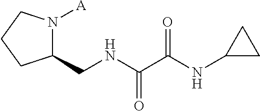

N(C(═O)C(═O)NC[C@@H]1N(*)CCC1)C1CC1

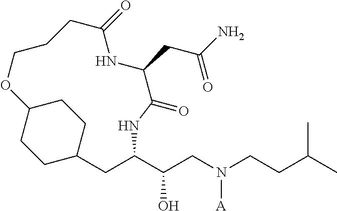

O[C@@H](CN(CCC(C)C)*)[C@H]1NC(═O)[C@H](CC(═O)N)NC(═O)CCCOC2CCC(CC2)C1

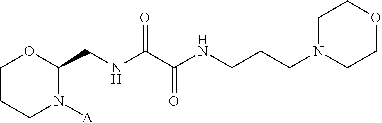

N(C(—O)C(—O)NC[C@@H]1OCCCN1*)CCCN1CCOCC1

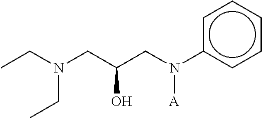

O[C@@H](CN(CC)CC)CN(c1ccccc1)*

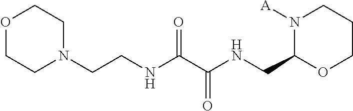

N(C(═O)C(═O)NC[C@@H]1OCCCN1*)CCN1CCOCC1

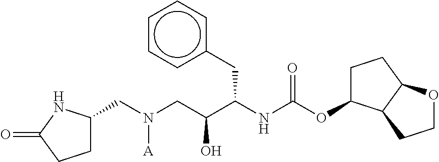

O[C@@H](CN(C[C@H]1NC(═O)CC1)*)[C@@H](NC(═O)O[C@H]1CC[C@H]2OCC
[C@@H]12)Cc1ccccc1

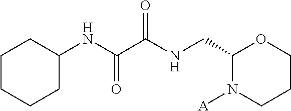

N(C(═O)C(═O)NC[C@@H]1OCCCN1*)C1CCCCC1

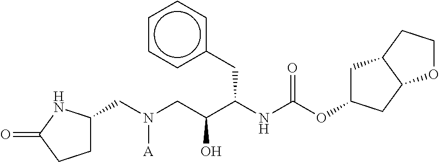

O[C@@H](CN(C[C@H]1NC(═O)CC1)*)[C@@H](NC(═O)O[C@@H]1C[C@@H]2CCO
[C@@H]2C1)Cc1ccccc1

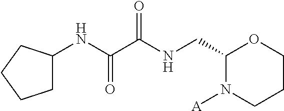

N(C(—O)C(—O)NC[C@@H]1OCCCN1*)C1CCCC1

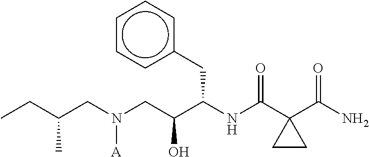

O[C@@H](CN(C[C@H](C)CC)*)[C@@H](NC(═O)C1(C(═O)N)CC1)Cc1ccccc1

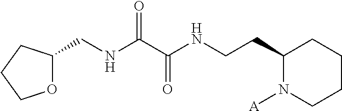

N(C(═O)C(═O)NC[C@@H]1OCCC1)CC[C@@H]1N(*)CCCC1

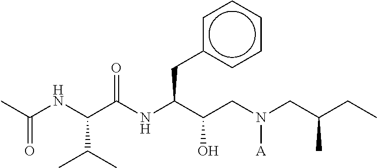

O[C@@H](CN(C[C@H](C)CC)*)[C@@H](NC(═O)[C@@H]NC(═O)C)C(C)C)Cc1ccccc1

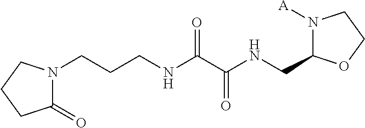

N(C(═O)C(═O)NC[C@@H]1OCCN1*)CCCN1C(═O)CCC1

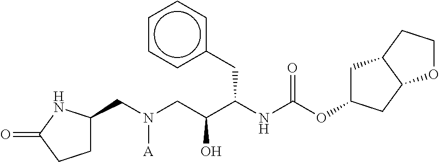

O[C@@H](CN(C[C@@H]1NC(═O)CC1)*)[C@@H](NC(═O)O[C@@H]1C[C@@H]2CCO
[C@@H]2C1)Cc1ccccc1

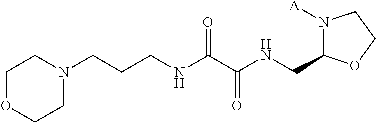

N(C(—O)C(—O)NC[C@@H]1OCCCN1*)CCCN1CCOCC1

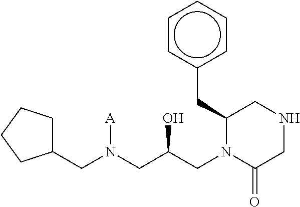

O[C@@H](CN1C(═O)CNC[C@@H]1Cc1ccccc1)CN(CC1CCCC1)*

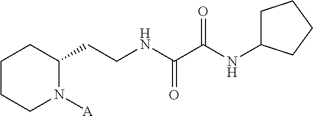

N(C(═O)C(═O)NC1CCCC1)CC[C@@H]1N(*)CCCC1

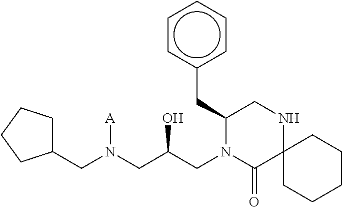

O[C@@H](CN1C(═O)C2(NC[C@@H]1Cc1ccccc1)CCCCC2)CN(CC1CCCC1)*

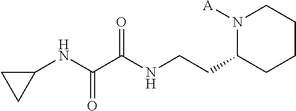

N(C(═O)C(═O)NC1CC1)CC[C@@H]1N(*)CCCC1

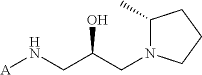

O[C@@H](CN1CCC[C@H]1CCN*

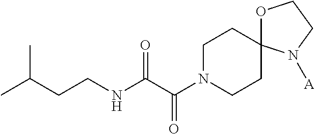

N(C(—O)C(—O)N1CCC2(OCCN2*)CC1)CCC(C)C

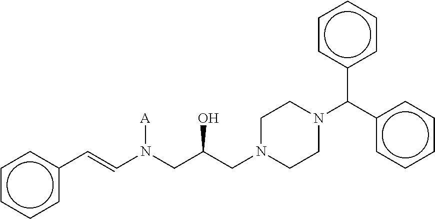

O[C@@H](CN1CCN(C(c2ccccc2)c2ccccc2)CC1)CN(/C═C/c1ccccc1)*

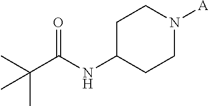

N(C(═O)C(C)(C)(C)C1CCN(*)CC1

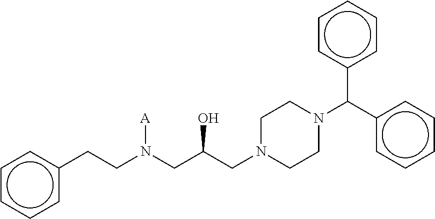

O[C@@H](CN1CCN(C(c2ccccc2)c2ccccc2)CC1)CN(CCc1ccccc1)*

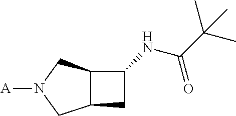

N(C(═O)C(C)(C)C)[C@@H]1C[C@H]2CN(*)C[C@@H]12

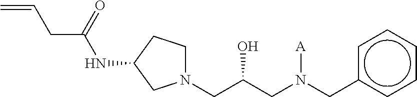

O[C@@H](CN1CC[C@@H](NC(═O)CC═C)C1)CN(Cc1ccccc1)*

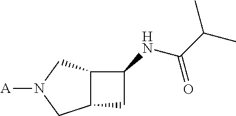

N(C(—O)C(C)C)[C@H]1C[C@@H]2CN(*)C[C@H]12

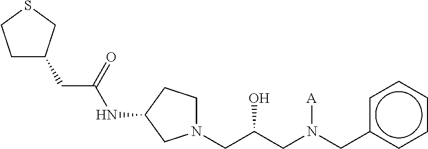

O[C@@H](CN1CC[C@@H](NC(═O)C[C@H]2CSCC2)C1)CN(Cc1ccccc1)*

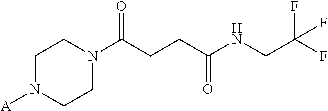

N(C(═O)CCC(═O)N1CCN(*)CC1)CC(F)(F)F

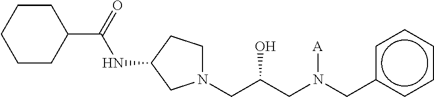

O[C@@H](CN1CC[C@@H](NC(═O)C2CCCCC2)C1)CN(Cc1ccccc1)*

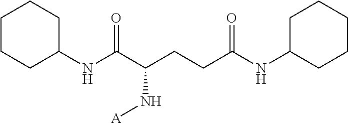

N(C(═O)CC[C@@H](C(═O)NC1CCCCC1)N*)C1CCCCC1

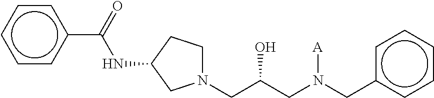

O[C@@H](CN1CC[C@@H](NC(═O)c2ccccc2)C1)CN(Cc1ccccc1)*

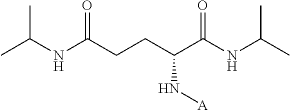

N(C(—O)CC[C@H](C(—O)NC(C)C)N*)C(C)C

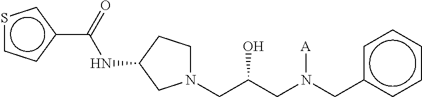

O[C@@H](CN1CC[C@@H](NC(═O)c2cscc2)C1)CN(Cc1ccccc1)*

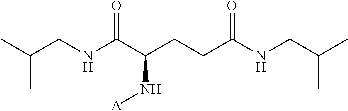

N(C(═O)CC[C@H](C(═O)NCC(C)C)N*)CC(C)C

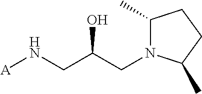

O[C@@H](CN1[C@H](C)CC[C@H]1C)CN*

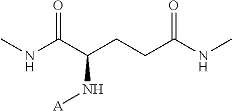

N(C(═O)CC[C@H](C(═O)NC)N*)C

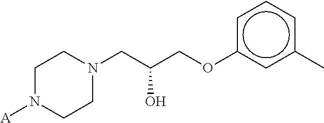

O[C@@H](COc1cccc(C)c1)CN1CCN(*)CC1

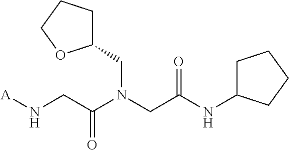

N(C(—O)CN(C(—O)CN*)C[C@@H]1OCCC1)C1CCCC1

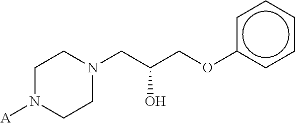

O[C@@H](COc1ccccc1)CN1CCN(*)CC1

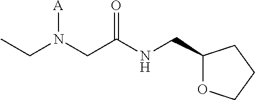

N(C(═O)CN(CC)*)C[C@@H]1OCCC1

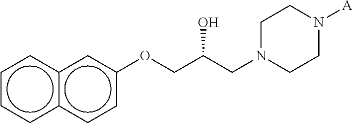

O[C@@H](COc1ccc2ccccc2c1)CN1CCN(*)CC1

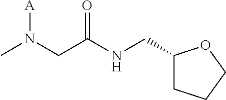

N(C(═O)CN(C)*)C[C@@H]1OCCC1

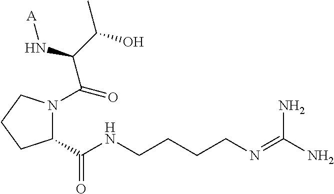

O[C@@H](C)[C@@H](C(═O)N1CCC[C@H]1C(═O)NCCCCN═C(N)N)N*

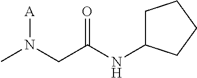

N(C(—O)CN(C)*)C1CCCC1

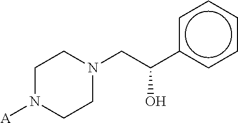

O[C@@H](c1ccccc1)CN1CCN(*)CC1

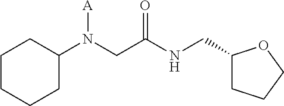

N(C(═O)CN(C1CCCCC1)*)C[C@@H]1OCCC1

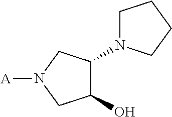

O[C@H]1CN(*)C[C@@H]1N1CCCC1

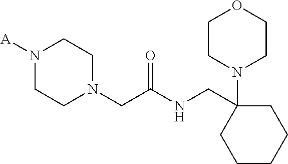

N(C(═O)CN1CCN(*)CC1)CC1(N2CCOCC2)CCCCC1

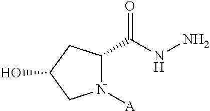

O[C@H]1CN(*)[C@@H](C(═O)NN)C1

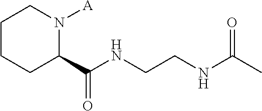

N(C(—O)C)CCNC(—O)[C@@H]1N(*)CCCC1

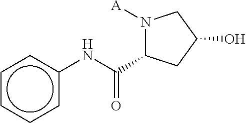

O[C@H]1CN(*)[C@@H](C(═O)Nc2ccccc2)C1

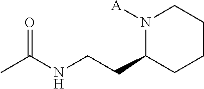

N(C(═O)C)CC[C@H]1N(*)CCCC1

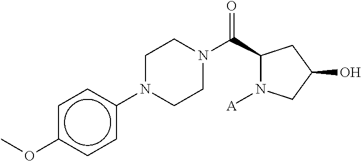

O[C@H]1CN(*)[C@@H](C(═O)N2CCN(c3ccc(OC)cc3)CC2)C1

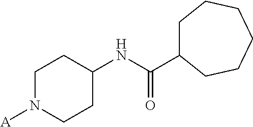

N(C(═O)C1CCCCCC1)C1CCN(*)CC1

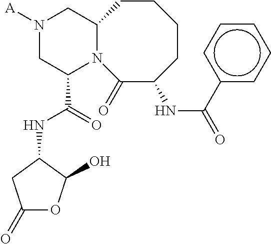

O[C@H]1OC(═O)C[C@@H]1NC(═O)1N2C(═O)[C@@H](NC(═O)c3ccccc3)
CCCC[C@H]2CN(*)C1

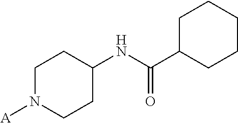

N(C(—O)C1CCCCCC1)C1CCN(*)CC1

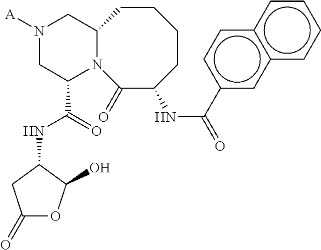

O[C@H]1OC(═O)C[C@@H]1NC(═O)1N2C(═O)[C@@H](NC(═O)c3ccc4ccccc4c3)
CCCC[C@H]2CN(*)C1

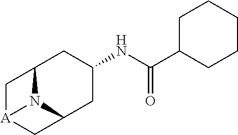

N(C(═O)C1CCCCC1)[C@@H]1C[C@H]2N(*)[C@H](CCC2)C1

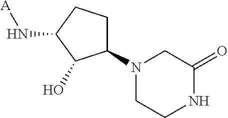

O[C@H]1[C@H](N2CC(═O)NCC2)CC[C@H]1N*

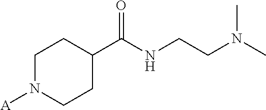

N(C(═O)C1CCN(*)CC1)CCN(C)C

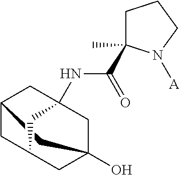

O[C@@]12C[C@H]3C[C@@H](C1)C[C@](NC(═O)[C@]1(C)N(*)CCC1)(C3)C2

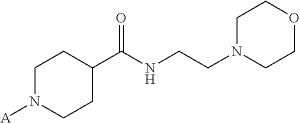

N(C(—O)C1CCN(*)CC1)CCN1CCOCC1

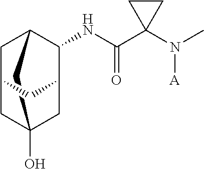

O[C@@]12C[C@H]3C[C@@H](C1)[C@H](NC(═O)C1(N(C)*)CC1)[C@H](C3)C2

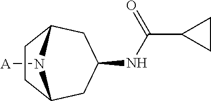

N(C(═O)C1CC1)[C@H]1C[C@H]2N(*)[C@H](CC2)C1

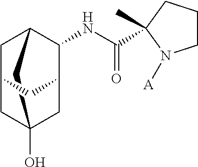

O[C@@]12C[C@H]3C[C@@H](C1)[C@H](NC(═O)[C@@]1(C)N(*)CCC1)[C@H](C3)C2

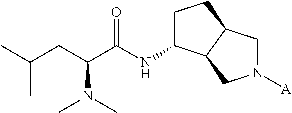

N(C(═O)[C@@H](N(C)C)CC(C)C)[C@@H]1CC[C@H]2CN(*)C[C@@H]12

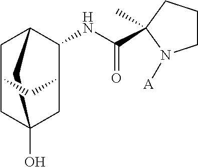

O[C@@]12C[C@H]3C[C@@H](C1)[C@H](NC(═O)[C@]1(C)N(*)CCC1)[C@H](C3)C2

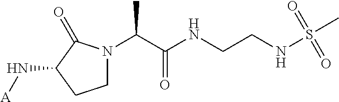

N(C(—O)[C@@H](N1C(—O)[C@@H](N*)CC1)C)CCNS(—O)(—O)C

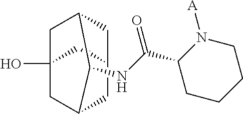

O[C@@]12C[C@H]3C[C@@H](C1)[C@H](NC(═O)[C@@H]1N(*)CCCC1)[C@H](C3)C2

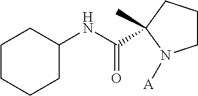

N(C(═O)[C@@]1(C)N(*)CCC1)C1CCCCC1

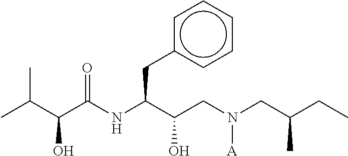

O[C@H](C(═O)N[C@@H](Cc1ccccc1)[C@@H](O)CN(C[C@H](C)CC)*)C(C)C

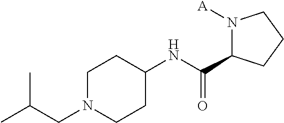

N(C(═O)[C@H]1N(*)CCC1)C1CCN(CC(C)C)CC1

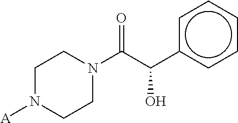

O[C@H](C(═O)N1CCN(*)CC1)c1ccccc1

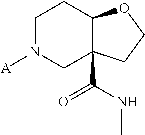

N(C(—O)[C@@]12CN(*)CC[C@H]1OCC2)C

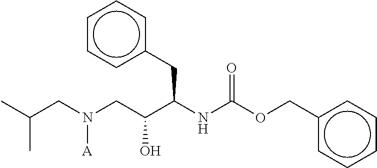

O[C@H](CN(CC(C)C)*)[C@H](NC(═O)OCc1ccccc1)Cc1ccccc1

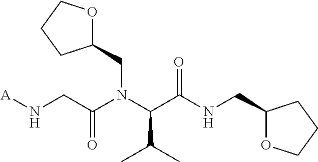

N(C(═O)[C@H](N(C(═O)CN*)C[C@@H]1OCCC1)C(C)C)C[C@@H]1OCCC1

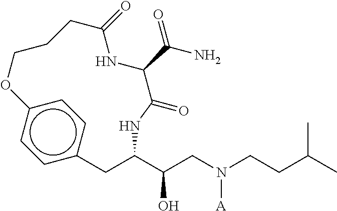

O[C@H](CN(CCC(C)C)*)[C@H]1NC(═O)[C@H](C(═O)N)NC(═O)CCCOc2ccc(cc2)C1

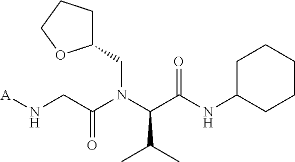

N(C(═O)[C@H](N(C(═O)CN*)C[C@@H]1OCCC1)C(C)C)C1CCCC1

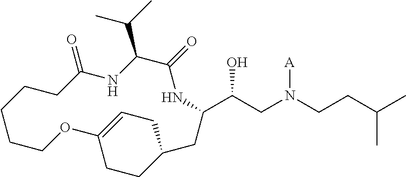

O[C@H](CN(CCC(C)C)*)[C@H]1NC(═O)[C@H](C(C)C)NC(═O)
CCCCCOC2═CC[C@@H](CC2)C1

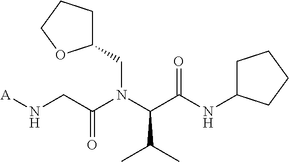

N(C(—O)[C@H](N(C(—O)CN*)C[C@@H]1OCCC1)C(C)C)C1CCCC1

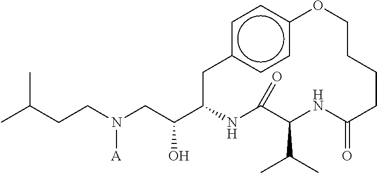

O[C@H](CN(CCC(C)C)*)[C@H]1NC(═O)[C@H](C(C)C)NC(═O)CCCOOc2ccc(cc2)C1

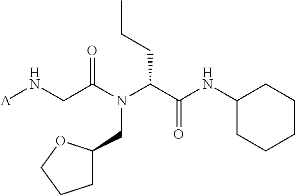

N(C(═O)[C@H](N(C(═O)CN*)C[C@@H]1OCCC1)CCC)C1CCCCC1

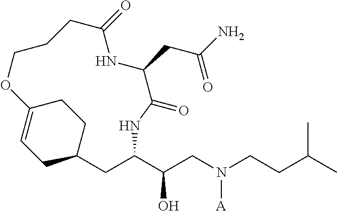

O[C@H](CN(CCC(C)C)*)[C@H]1NC(═O)[C@H](CC(═O)N)NC(═O)
CCCOC2═CC[C@@H](CC2)C1

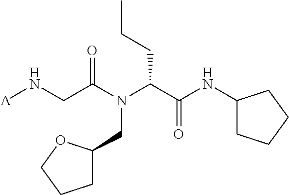

N(C(═O)[C@H](N(C(═O)CN*)C[C@@H]1OCCC1)CCC)C1CCCC1

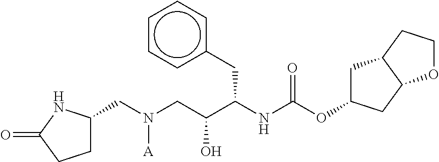

O[C@H](CN(C[C@H]1NC(═O)CC1)*)[C@@H](NC(═O)O[C@@H]1C[C@@H]2
CCO[C@@H]2C1)Cc1ccccc1

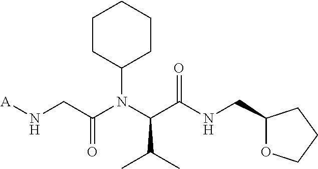

N(C(—O)[C@H](N(C(—O)CN*)C1CCCCC1)C(C)C)C[C@@H]1OCCC1

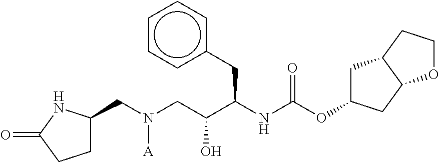

O[C@H](CN(C[C@@H]1NC(═O)CC1)*)[C@H](NC(═O)O[C@@H]1C[C@@H]2
CCO[C@@H]2C1)Cc1ccccc1

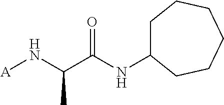

N(C(═O)[C@H](N*)C)C1CCCCCC1

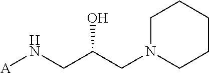

O[C@H](CN1CCCCC1)CN*

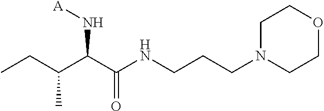

N(C(═O)[C@H](N*)[C@H](C)CC)CCCN1CCOCC1

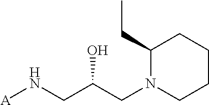

O[C@H](CN1CCCC[C@@H]1CC)CN*

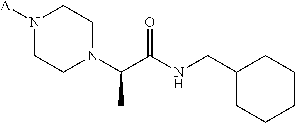

N(C(—O)[C@H](N1CCN(*)CC1)C)CC1CCCCC1

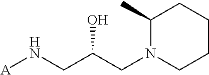

O[C@H](CN1CCCC[C@@H]1C)CN*

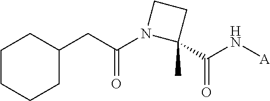

N(C(═O)[C@]1(C)N(C(═O)CC2CCCCC2)CC1)*

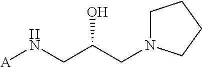

O[C@H](CN1CCCC1)CN*

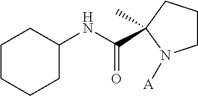

N(C(═O)[C@]1(C)N(*)CCC1)C1CCCCC1

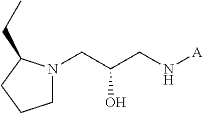

O[C@H](CN1CCC[C@H]1CC)CN*

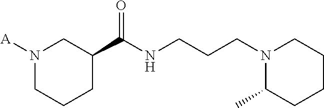

N(C(—O)[C@@H]1CN(*)CCC1)CCCN1CCCC[C@@H]1C

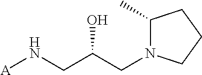

O[C@H](CN1CCC[C@H]1C)CN*

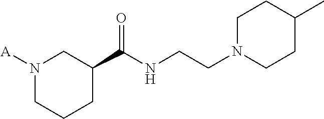

N(C(═O)[C@@H]1CN(*)CCC1)CCN1CCC(C)CC1

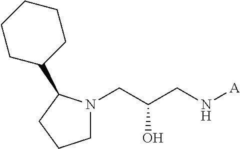

O[C@H](CN1CCC[C@H]1C1CCCCC1)CN*

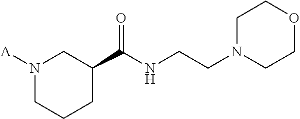

N(C(═O)[C@@H]1CN(*)CCC1)CCN1CCOCC1

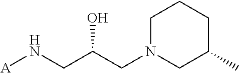

O[C@H](CN1CCC[C@H](C)C1)CN*

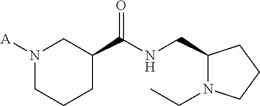

N(C(—O)[C@@H]1CN(*)CCC1)C[C@@H]1N(CC)CCC1

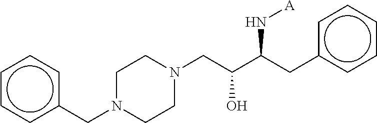

O[C@H](CN1CCN(Cc2ccccc2)CC1)[C@@H](N*)Cc1ccccc1

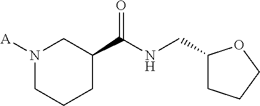

N(C(═O)[C@@H]1CN(*)CCC1)C[C@@H]1OCCC1

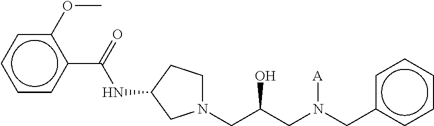

O[C@H](CN1CC[C@@H](NC(═O)c2ccccc2OC)C1)CN(Cc1ccccc1)*

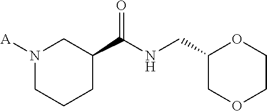

N(C(═O)[C@@H]1CN(*)CCC1)C[C@@H]1OCCOC1

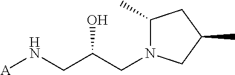

O[C@H](CN1C[C@H](C)C[C@H]1C)CN*

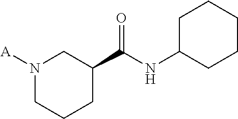

N(C(—O)[C@@H]1CN(*)CCC1)C1CCCCC1

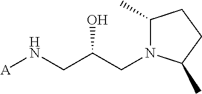

O[C@H](CN1[C@H](C)CC[C@H]1C)CN*

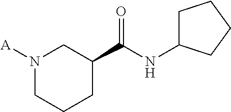

N(C(═O)[C@@H]1CN(*)CCC1)C1CCCC1

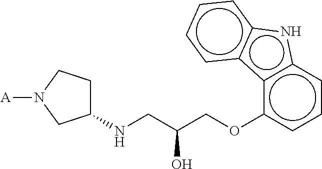

O[C@H](COc1cccc2c1c1ccccc1[nH]2)CN[C@@H]1CN(*)CC1

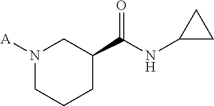

N(C(═O)[C@@H]1CN(*)CCC1)C1CC1

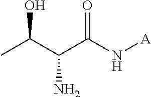

O[C@H](C)[C@H](C(═O)N*)N

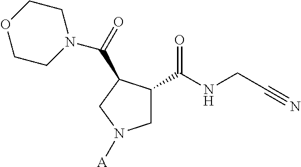

N(C(—O)[C@@H]1CN(*)C[C@H]1C(—O)N1CCOCC1)CC#N

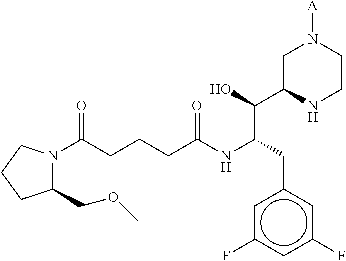

O[C@H]([C@@H](NC(═O)CCCC(═O)N1CCC[C@@H]1COC)
Cc1cc(F)cc(F)c1)[C@@H]1NCCN(*)C1

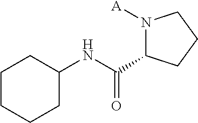

N(C(═O)[C@@H]1N(*)CCC1)C1CCCCC1

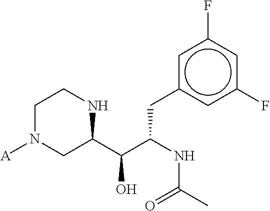

O[C@H]([C@@H](NC(═O)C)Cc1cc(F)cc(F)c1)[C@@H]1NCCN(*)C1

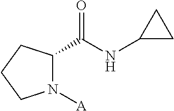

N(C(═O)[C@@H]1N(*)CCC1)C1CC1

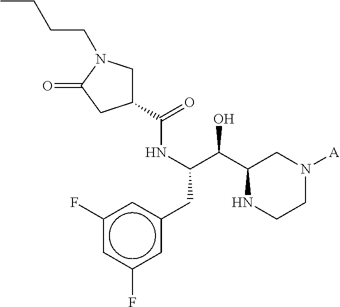

O[C@H]([C@@H](NC(═O)[C@@H]1CC(═O)N(CCCC)C1)
Cc1cc(F)cc(F)c1)[C@@H]1NCCN(*)C1

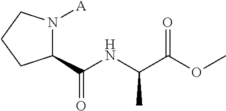

N(C(—O)[C@@H]1N(*)CCC1)[C@@H](C(—O)OC)C

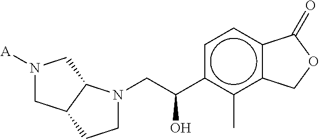

O[C@H](c1ccc2c(c1C)COC2═O)CN1CC[C@@H]2CN(*)C[C@H]12

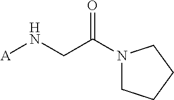

N(CC(═O)N1CCCC1)*

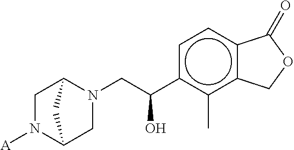

O[C@H](c1ccc2c(c1C)COC2═O)CN1C[C@H]2N(*)C[C@@H]1C2

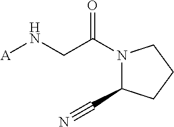

N(CC(═O)N1CCC[C@H]1C#N)*

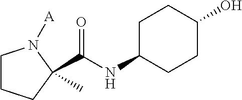

O[C@@H]1CC[C@@H](NC(═O)[C@]2(C)N(*)CCC2)CC1

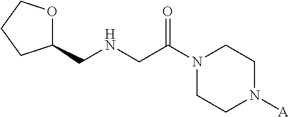

N(CC(—O)N1CCN(*)CC1)C[C@@H]1OCCC1

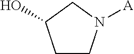

O[C@@H]1CN(*)CC1

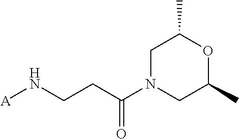

N(CCC(═O)N1C[C@H](C)O[C@@H](C)C1)*

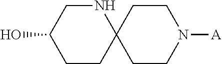

O[C@@H]1CNC2(CCN(*)CC2)CC1

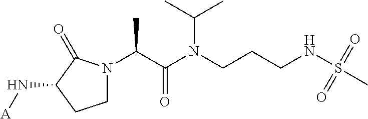

N(CCCN(C(═O)[C@@H](N1C(═O)[C@@H](N*)CC1)C)C(C)C)S(═O)(═O)C

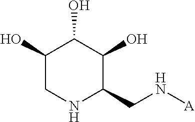

O[C@@H]1[C@@H](O)[C@@H](CN*)NC[C@H]1O

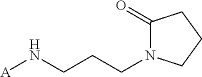

N(CCCN1C(—O)CCC1)*

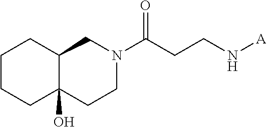

O[C@H]12CCN(C(═O)CCN*)C[C@@H]1CCCC2

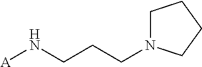

N(CCCN1CCCC1)*

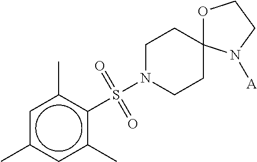

c1c(C)cc(C)c(S(═O)(═O)N2CCC3(OCCN3*)CC2)c1C

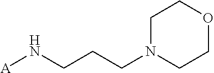

N(CCCN1CCOCC1)*

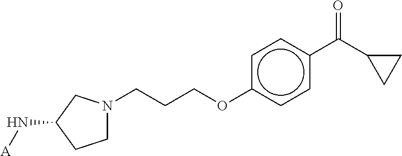

c1cc(C(═O)C2CC2)ccc1OCCCN1CC[C@H](N*)C1

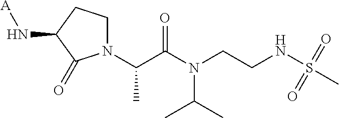

N(CCN(C(—O)[C@@H](N1C(—O)[C@@H](N*)CC1)C)C(C)C)S(—O)(—O)C

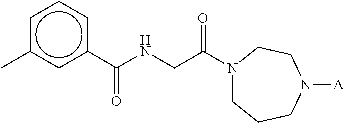

c1cc(C(═O)NCC(═O)N2CCN(*)CCC2)cc(C)c1

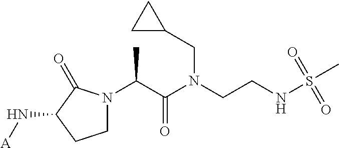

N(CCN(C(═O)[C@@H](N1C(═O)[C@@H](N*)CC1)C)CC1CC1)S(═O)(═O)C

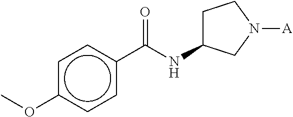

c1cc(C(═O)N[C@@H]2CN(*)CC2)ccc1OC

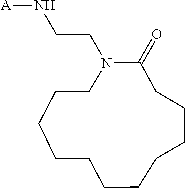

N(CCN1C(═O)CCCCCCCCCCC1)*

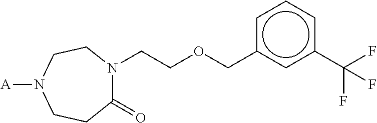

c1cc(C(F)(F)F)cc(COCCN2C(═O)CCN(*)CC2)c1

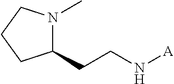

N(CC[C@@H]1N(C)CCC1)*

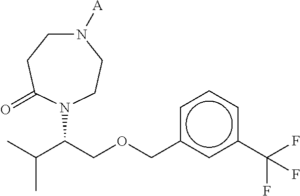

c1cc(C(F)(F)F)cc(COC[C@@H](N2C(═O)CCN(*)CC2)C(C)C)c1

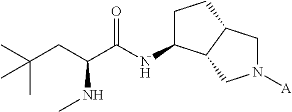

N(C)[C@H](C(═O)N[C@H]1CC[C@@H]2CN(*)C[C@H]12)CC)(C)(C)C

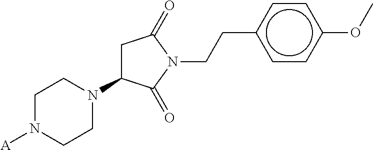

c1cc(CCN2C(═O)C[C@H](N3CCN(*)CC3)C2═O)ccc1OC

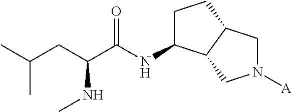

N(C)[C@H](C(═O)N[C@H]1CC[C@@H]2CN(*)C[C@H]12)CC)(C)C

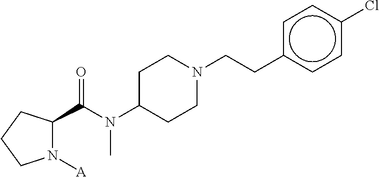

c1cc(CCN2CCCC(N(C(═O)[C@H]3N(*)CCC3)C)CC2)ccc1C1

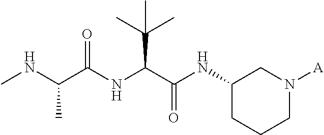

N(C)[C@H](C(—O)N[C@H](C(—O)N[C@@H]1CN(*)CCC1)C(C)(C)C)C

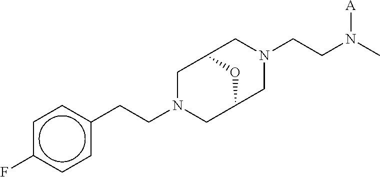

c1cc(CCN2C[C@@H]3O[C@H](C2)CN(CCN(C)*)C3)ccc1F

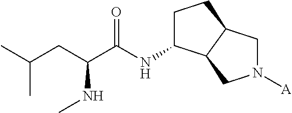

N(C)[C@H](C(═O)N[C@@H]1CC[C@H]2CN(*)C[C@@H]12)CC(C)C

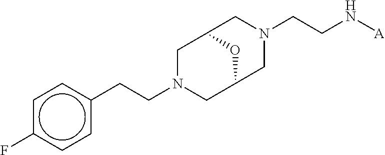

c1cc(CCN2C[C@@H]3O[C@H](C2)CN(CCN*)C3)ccc1F

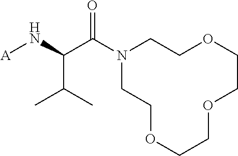

N([C@@H](C(═O)N1CCOCCOCCOCC1)C(C)C)*

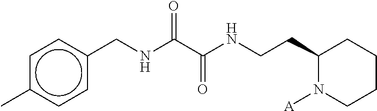

c1cc(CNC(═O)C(═O)NCC[C@@H]2N(*)CCCC2)ccc1C

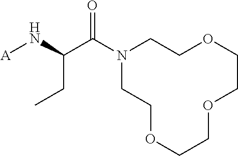

N([C@@H](C(—O)N1CCOCCOCCOCC1)CC)*

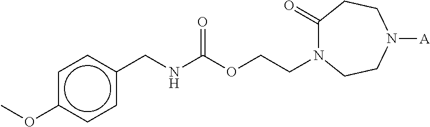

c1cc(CNC(═O)OCCN2C(═O)CCN(*)CC2)ccc1OC

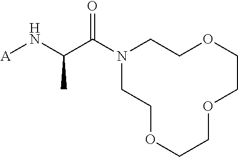

N([C@@H](C(═O)N1CCOCCOCCOCC1)C)*

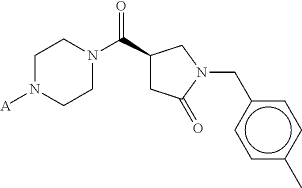

c1cc(CN2C(═O)C[C@@H](C(═O)N3CCN(*)CC3)C2)ccc1C

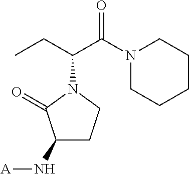

N([C@H]1C(═O)N([C@@H](C(═O)N2CCCCC2)CC)CC1)*

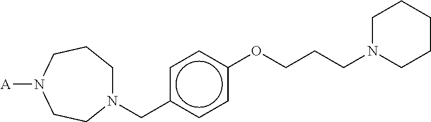

c1cc(CN2CCN(*)CCC2)ccc1OCCCN1CCCCC1

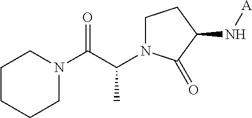

N([C@H]1C(—O)N([C@@H](C(—O)N2CCCCC2)C)CC1)*

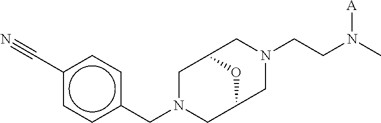

c1cc(CN2C[C@@H]3O[C@H](C2)CN(CCN(C)*)C3)ccc1C#N

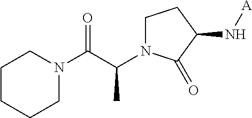

N([C@H]1C(═O)N([C@H](C(═O)N2CCCCC2)C)CC1)*

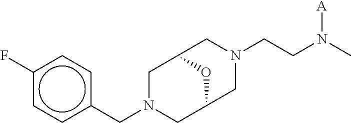

c1cc(CN2C[C@@H]3O[C@H](C2)CN(CCN(C)*)C3)ccc1F

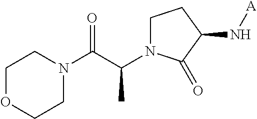

N([C@H]1C(═O)N([C@H](C(═O)N2CCOCC2)C)CC1)*

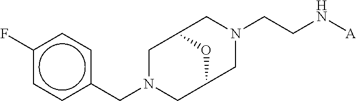

c1cc(CN2C[C@@H]3O[C@H](C2)CN(CCN*)C3)ccc1F

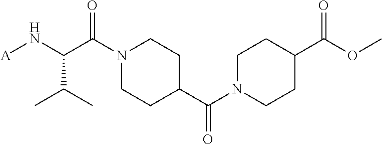

N([C@H](C(—O)N1CCC(C(—O)N2CCC(C(—O)OC)CC2)CC1)C(C)C)*

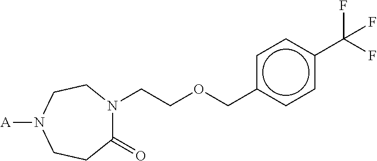

c1cc(COCCN2C(═O)CCN(*)CC2)ccc1C(F)(F)F

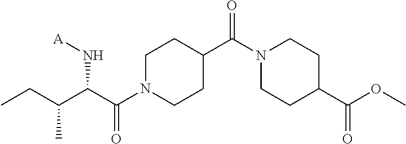

N([C@H](C(═O)N1CCC(C(═O)N2CCC(C(═O)OC)CC2)CC1)[C@H](C)CC)*

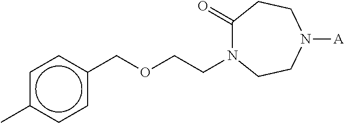

c1cc(COCCN2C(═O)CCN(*)CC2)ccc1C

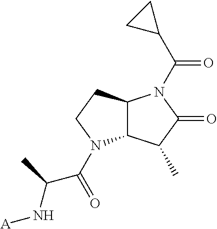

N([C@H](C(═O)N1CC[C@H]2N(C(═O)C3CC3)C(═O)[C@H](C)[C@H]12)C)*

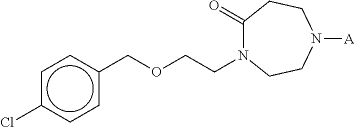

c1cc(COCCN2C(═O)CCN(*)CC2)ccc1C1

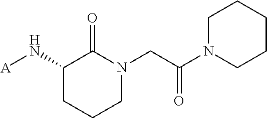

N([C@@H]1C(—O)N(CC(—O)N2CCCCC2)CCC1)*

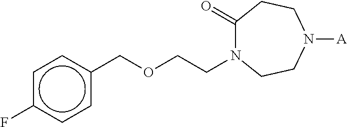

c1cc(COCCN2C(═O)CCN(*)CC2)ccc1F

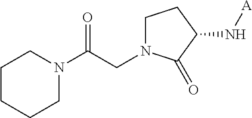

N([C@@H]1C(═O)N(CC(═O)N2CCCCC2)CC1)*

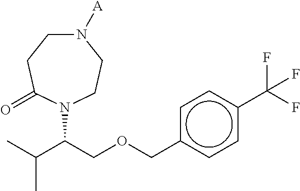

c1cc(COC[C@@H](N2C(═O)CCN(*)CC2)C(C)C)ccc1C(F)(F)F

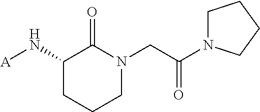

N([C@@H]1C(═O)N(CC(═O)N2CCCC2)CCC1)*

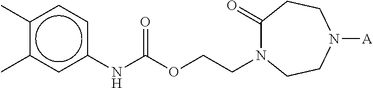

c1cc(C)c(C)cc1NC(═O)OCCN1C(═O)CCN(*)CC1

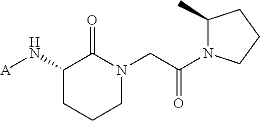

N([C@@H]1C(—O)N(CC(—O)N2CCC[C@@H]2C)CCC1)*

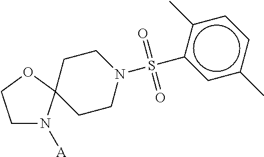

c1cc(C)c(S(═O)(═O)N2CCC3(OCCN3*)CC2)cc1C

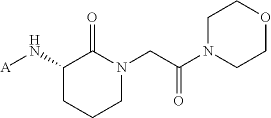

N([C@@H]1C(═O)N(CC(═O)N2CCOCC2)CCC1)*

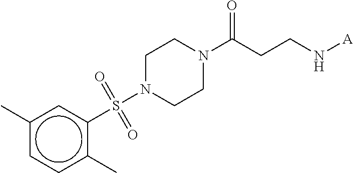

c1cc(C)c(S(═O)(═O)N2CCN(C(═O)CCN*)CC2)cc1C

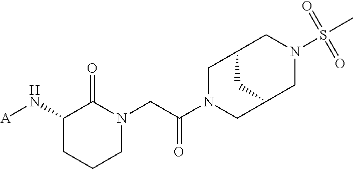

N([C@@H]1C(═O)N(CC(═O)N2C[C@H]3CN(S(═O)(═O)C)C[C@@H](C2)C3)CCC1)*

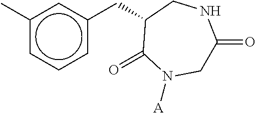

c1cc(C)cc(C[C@H]2C(═O)N(*)CC(═O)NC2)c1

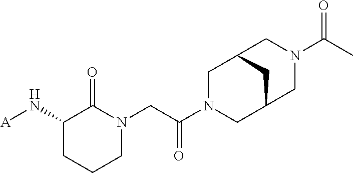

N([C@@H]1C(—O)N(CC(—O)N2C[C@@H]3CN(C(—O)C)C[C@H](C2)C3)CCC1)*

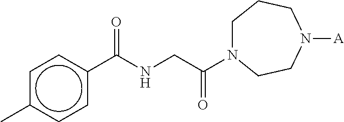

c1cc(C)ccc1C(═O)NCC(═O)N1CCN(*)CCC1

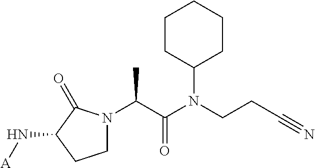

N([C@@H]1C(═O)N([C@H](C(═O)N(CCC#N)C2CCCCC2)C)CC1)*

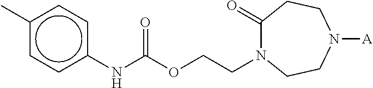

c1cc(C)ccc1NC(═O)OCCN1C(═O)CCN(*)CC1

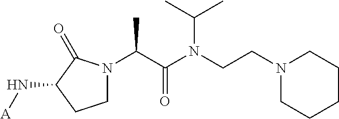

N([C@@H]1C(═O)N([C@H](C(═O)N(CCN2CCCCC2)C(C)C)C)CC1)*

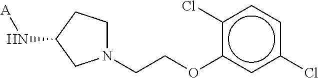

c1cc(C1)c(OCCN2CC[C@@H](N*)C2)cc1C1

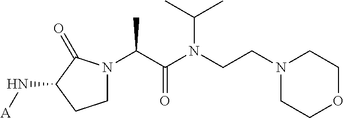

N([C@@H]1C(—O)N([C@H](C(—O)N(CCN2CCOCC2)C(C)C)C)CC1)*

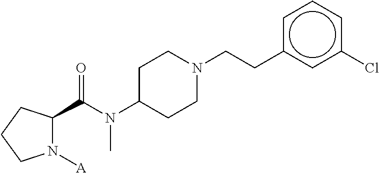

c1cc(C1)cc(CCN2CCC(N(C(═O)[C@H]3N(*)CCC3)C)CC2)c1

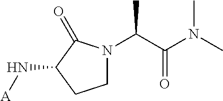

N([C@@H]1C(═O)N([C@H](C(═O)N(C)C)C)CC1)*

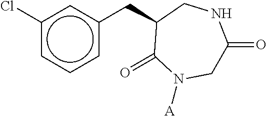

c1cc(C1)cc(C[C@@H]2C(═O)N(*)CC(═O)NC2)c1

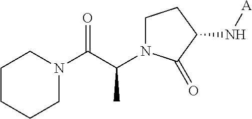

N([C@@H]1C(═O)N([C@H](C(═O)N2CCCCC2)C)CC1)*

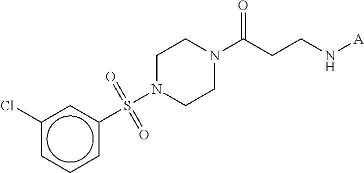

c1cc(C1)cc(S(═O)(═O)N2CCN(C(═O)CCN*)CC2)c1

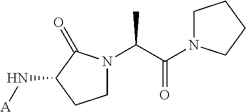

N([C@@H]1C(—O)N([C@H](C(—O)N2CCCC2)C)CC1)*

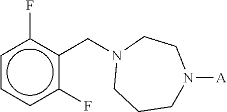

c1cc(F)c(CN2CCN(*)CCC2)c(F)c1

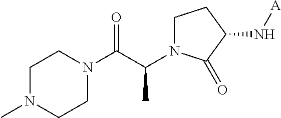

N([C@@H]1C(═O)N([C@H](C(═O)N2CCN(C)CC2)C)CC1)*

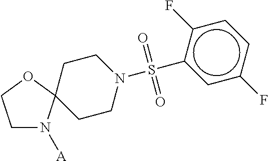

c1cc(F)cc(S(═O)(═O)N2CCC3(OCCN3*)CC2)cc1F

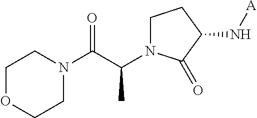

N([C@@H]1C(═O)N([C@H](C(═O)N2CCOCC2)C)CC1)*

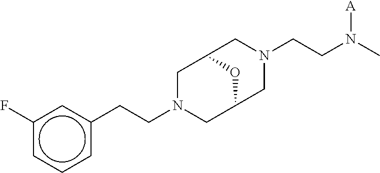

c1cc(F)cc(CCN2C[C@@H]3O[C@H](C2)CN(CCN(C)*)C3)c1

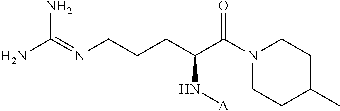

NC(—NCCC[C@@H](C(—O)N1CCC(C)CC1)N*)N

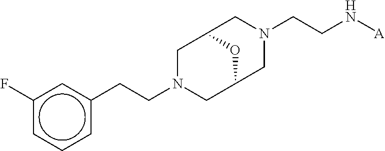

c1cc(F)cc(CCN2C[C@@H]3O[C@H](C2)CN(CCN*)C3)c1

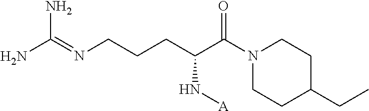

NC(═NCCC[C@H](C(═O)N1CCC(CC)CC1)N*)N

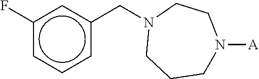

c1cc(F)cc(CN2CCN(*)CCC2)c1

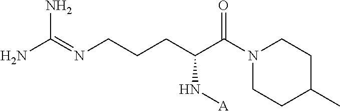

NC(═NCCC[C@H](C(═O)N1CCC(C)CC1)N*)N

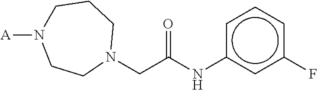

c1cc(F)cc(NC(═O)CN2CCN(*)CCC2)c1

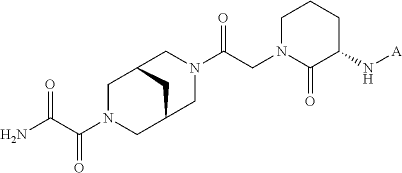

NC(—O)C(—O)N1C[C@@H]2CN(C(—O)CN3C(—O)[C@@H](N*)CC3)C[C@H](C1)C2

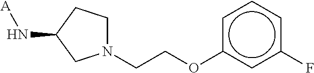

c1cc(F)cc(OCCN2CC[C@H](N*)C2)c1

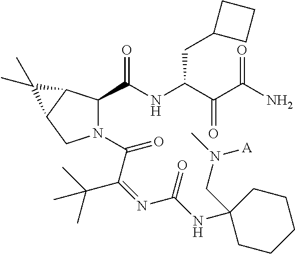

NC(═O)C(═O)[C@H](NC(═O)[C@H]1N(C(═O)/C(═N\C(═O)NC2(CN(C)*)CCCCC2)/
C(C)(C)C)C[C@@H]2C(C)(C)[C@H]12)CC1CCC1

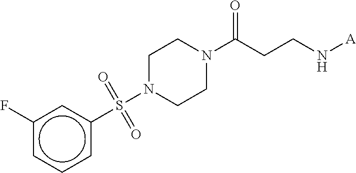

c1cc(F)cc(S(═O)(═O)N2CCN(C(═O)CCN*)CC2)c1

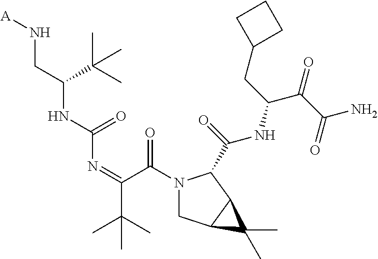

NC(═O)C(═O)[C@H](NC(═O)[C@H]1N(C(═O)/C(═N\C(═O)N[C@H](CN*)C(C)(C)C)/C
(C)(C)C)C[C@@H]2C(C)(C)[C@H]12)CC1CCC1

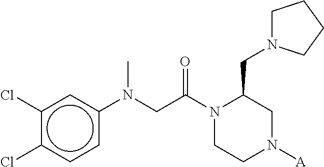

c1cc(N(C)CC(═O)N2CCN(*)C[C@@H]2CN2CCCC2)cc(C1)c1C1

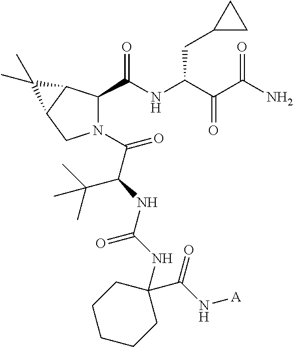

NC(═O)C(═O)[C@H](NC(═O)[C@H]1N(C(═O)[C@@H](NC(═O)NC2(C(═O)N*)CCCCC2)
C(C)(C)C)C[C@@H]2C(C)(C)[C@H]12)CC1CCC1

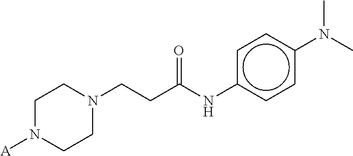

c1cc(N(C)C)ccc1NC(═O)CCN1CCN(*)CC1

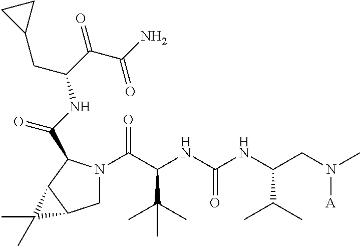

NC(═O)C(═O)[C@H](NC(═O)[C@H]1N(C(═O)[C@@H](NC(═O)N[C@H](CN(C)*)C(C)C)
C(C)(C)C)C[C@@H]2C(C)(C)[C@H]12)CC1CC1

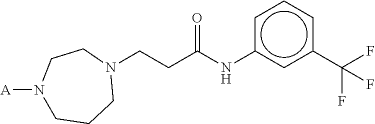

c1cc(NC(═O)CCN2CCN(*)CCC2)cc(C(F)(F)F)c1

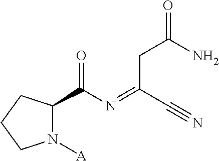

NC(═O)C/C(═N\C(═O)[C@H]1N(*)CCC1)/C#N

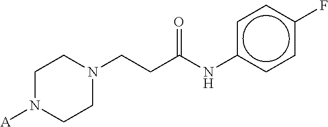

c1cc(NC(═O)CCN2CCN(*)CC2)ccc1F

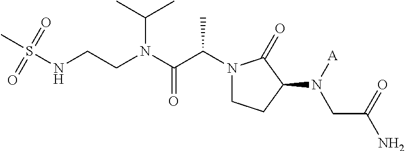

NC(—O)CN([C@@H]1C(—O)N([C@H](C(—O)N(CCNS(—O)(—O)C)C(C)C)C)CC1)*

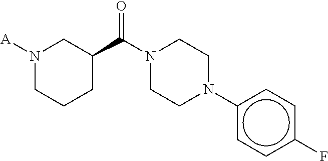

c1cc(N2CCN(C(═O)[C@@H]3CN(*)CCC3)CC2)ccc1F

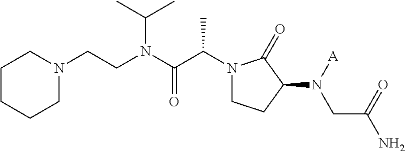

NC(═O)CN([C@@H]1C(═O)N([C@H](C(═O)N(CCN2CCCCC2)C(C)C)C)CC1)*

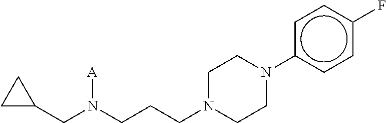

c1cc(N2CCN(CCCN(CC3CC3)*)CC2)ccc1F

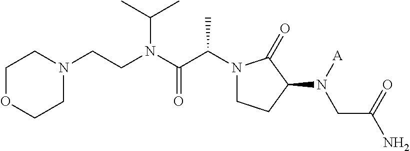

NC(═O)CN([C@@H]1C(═O)N([C@H](C(═O)N(CCN2CCOCC2)C(C)C)C)CC1)*

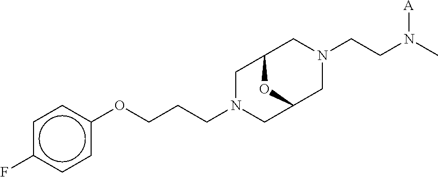

c1cc(OCCCN2C[C@@H]3O[C@@H](CN(CCN(C)*)C3)C2)ccc1F

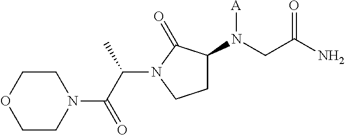

NC(—O)CN([C@@H]1C(—O)N([C@H](C(—O)N2CCOCC2)C)CC1)*

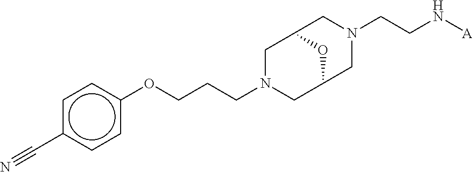

c1cc(OCCCN2C[C@@H]3O[C@@H](CN(CCN*)C3)C2)ccc1C#N

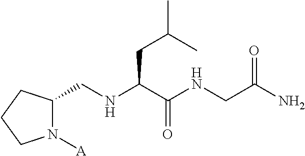

NC(═O)CNC(═O)([C@@H](NC[C@@H]1N(*)CCC1)CC(C)C

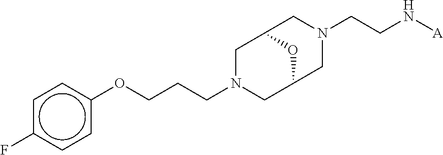

c1cc(OCCCN2C[C@@H]3O[C@@H](CN(CCN*)C3)C2)ccc1F

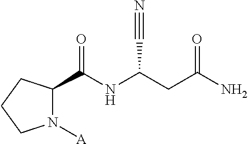

NC(═O)C[C@@H](C#N)NC(═O)[C@H]1N(*)CCC1

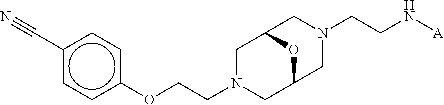

c1cc(OCCN2C[C@H]3O[C@@H](C2)CN(CCN*)C3)ccc1C#N

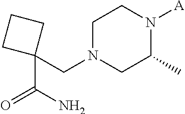

NC(—O)C1(CN2CCN(*)[C@H](C)C2)CCC1

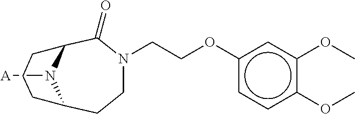

c1cc(OC)c(OC)cc1OCCN1C(═O)[C@@H]2N(*)[C@@H](CC1)CCC2

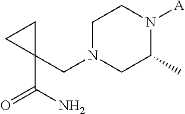

NC(═O)C1(CN2CCN(*)[C@H](C)C2)CC1

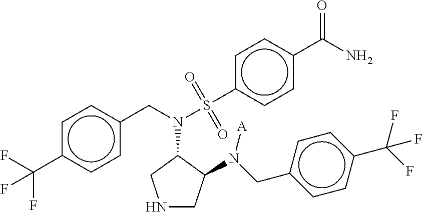

c1cc(S(═O)(═O)N(Cc2ccc(C(F)(F)F)cc2)[C@H]2CNC[C@@H]2N
(Cc2ccc(C(F)(F)F)cc2)*)ccc1C(═O)N

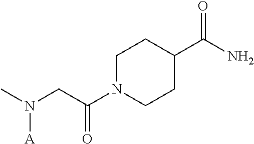

NC(═O)C1CCN(C(═O)CN(C)*)CC1

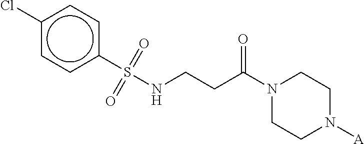

c1cc(S(═O)(═O)NCCC(═O)N2CCN(*)CC2)ccc1C1

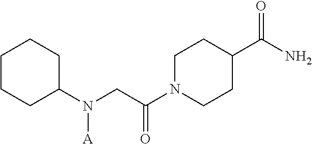

NC(—O)C1CCN(C(—O)CN(CC2CCCCC2)*)CC1

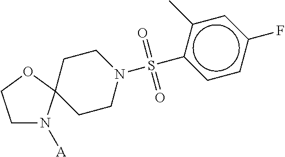

c1cc(S(═O)(═O)N2CCC3(OCCN3*)CC2)c(C)cc1F

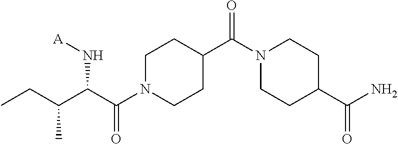

NC(═O)C1CCN(C(═O)C2CCN(C(═O)[C@@H](N*)[C@H](C)CC)CC2)CC1

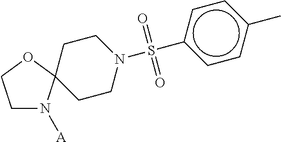

c1cc(S(═O)(═O)N2CCC3(OCCN3*)CC2)ccc1C

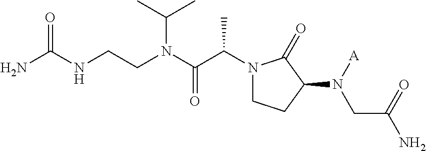

NC(═O)NCCN(C(═O)[C@@H](N1C(═O)[C@@H](N(CC(═O)N)*)CC1)C)C(C)C

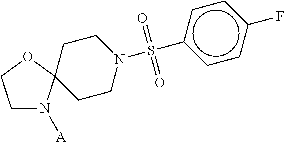

c1cc(S(═O)(═O)N2CCC3(OCCN3*)CC2)ccc1F

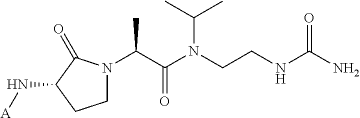

NC(—O)NCCN(C(—O)[C@@H](N1C(—O)[C@@H](N*)CC1)C)C(C)C

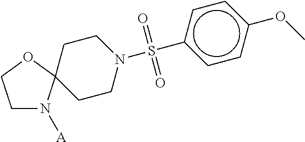

c1cc(S(═O)(═O)N2CCC3(OCCN3*)CC2)ccc1OC

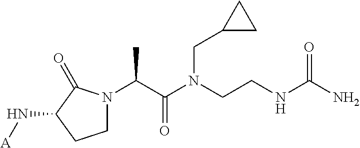

NC(═O)NCCN(C(═O)[C@@H](N1C(═O)[C@@H](N*)CC1)C)CC1CC1

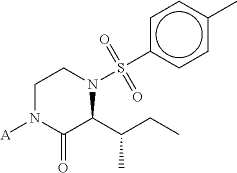

c1cc(S(═O)(═O)N2CCN(*)C(═O)[C@@H]2[C@@H](C)CC)ccc1C

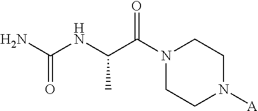

NC(═O)N[C@H](C(═O)N1CCN(*)CC1)C

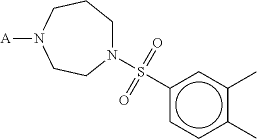

c1cc(S(═O)(═O)N2CCN(*)CCC2)cc(C)c1C

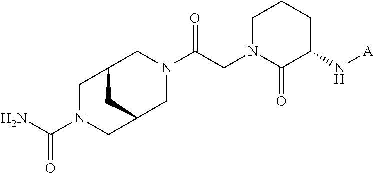

NC(—O)N1C[C@@H]2CN(C(—O)CN3C(—O)[C@@H](N*)CCC3)C[C@H](C1)C2

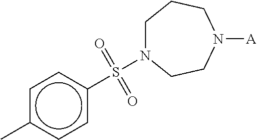

c1cc(S(═O)(═O)N2CCN(*)CCC2)ccc1C

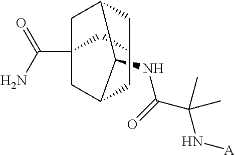

NC(═O)[C@@]12C[C@H]3C[C@@H](C1)[C@@H](NC(═O)(N*)(C)C)[C@H](C3)C2

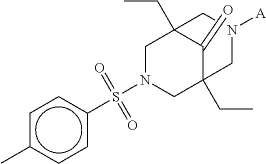

c1cc(S(═O)(═O)N2CC3(CC)C(═O)C(CC)(C2)CN(*)C3)ccc1C

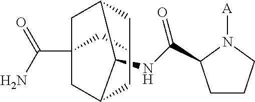

NC(═O)[C@@]12C[C@H]3C[C@@H](C1)[C@@H](NC(═O)[C@H]1N(*)CCC1)
[C@H](C3)C2

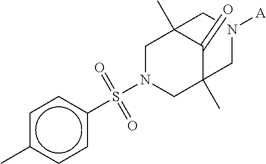

c1cc(S(═O)(═O)N2CC3(C)C(═O)C(C)(C2)CN(*)C3)ccc1C

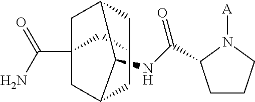

NC(—O)[C@@]12C[C@H]3C[C@@H](C1)[C@@H](NC(—O)[C@@H]1N(*)CCC1)
[C@H](C3)C2

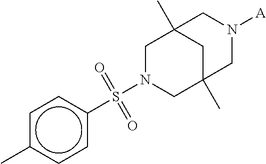

c1cc(S(═O)(═O)N2CC3(C)CN(*)CC(C)(C2)C3)ccc1C

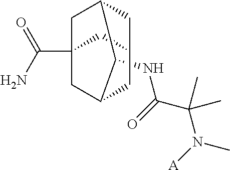

NC(═O)[C@@]12C[C@H]3C[C@@H](C1)[C@H](NC(═O)C(N(C)*)(C)C)[C@H](C3)C2

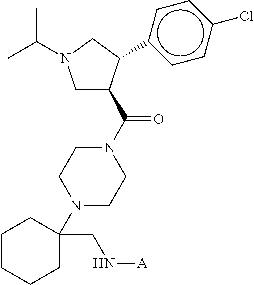

c1cc([C@H]2CN(C(C)C)C[C@@H]2C(═O)N2CCN(C3(CN*)CCCCC3)CC2)ccc1C1

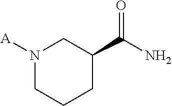

NC(═O)[C@@H]1CN(*)CCC1

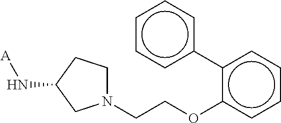

c1ccc(c2ccccc2OCCN2CC[C@@H](N*)C2)cc1

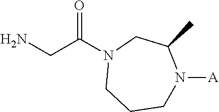

NCC(—O)N1CCCN(*)[C@H](C)C1

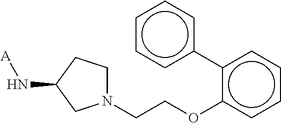

c1ccc(c2ccccc2OCCN2CC[C@H](N*)C2)cc1

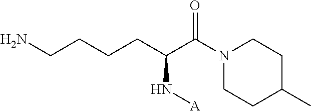

NCCCC[C@@H](C(═O)N1CCC(C)CC1)N*

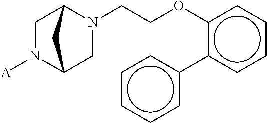

c1ccc(c2ccccc2OCCN2C[C@@H]3N(*)C[C@H]2C3)cc1

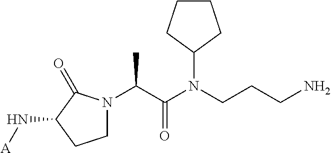

NCCCN(C(═O)[C@@H](N1C(═O)[C@@H](N*)CC1)C)C1CCCC1

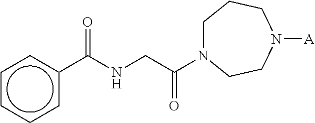

c1ccc(C(═O)NCC(═O)N2CCN(*)CCC2)cc1

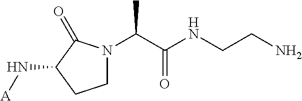

NCCNC(—O)[C@@H](N1C(—O)[C@@H](N*)CC1)C

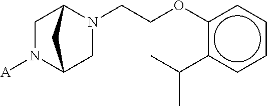

c1ccc(C(C)C)c(OCCN2C[C@@H]3N(*)C[C@H]2C3)c1

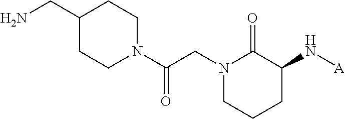

NCC1CCN(C(═O)CN2C(═O)[C@@H](N*)CCC2)CC1

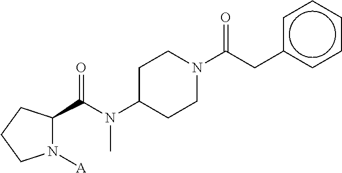

c1ccc(CC(═O)N2CCC(N(C(═O)[C@H]3N(*)CCC3)C)CC2)cc1

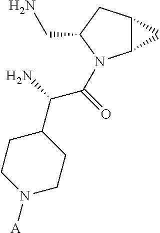

NC[C@H]1N(C(═O)[C@@H](N)C2CCN(*)CC2)[C@H]2C[C@H]2C1

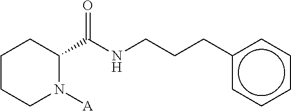

c1ccc(CCCNC(═O)[C@@H]2N(*)CCCC2)cc1

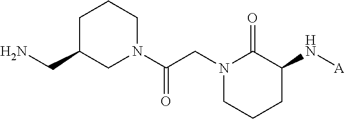

NC[C@@H]1CN(C(—O)CN2C(—O)[C@@H](N*)CCC2)CCC1

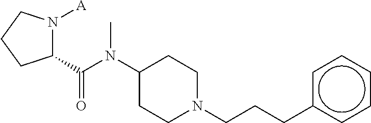

c1ccc(CCCN2CCC(N(C(═O)[C@H]3N(*)CCC3)C)CC2)cc1

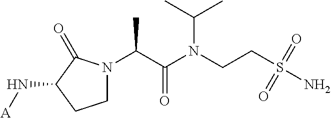

NS(═O)(═O)CCN(C(═O)[C@@H](N1C(═O)[C@@H](N*)CC1)C)C(C)C

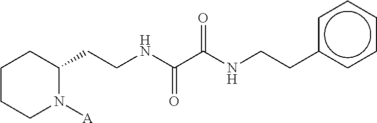

c1ccc(CCNC(═O)C(═O)NCC[C@@H]2N(*)CCCC2)cc1
A—NH₂
N*

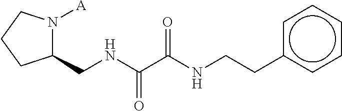

c1ccc(CCNC(═O)C(═O)NC[C@@H]2N(*)CCC2)cc1

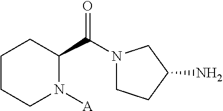

N[C@H]1CN(C(—O)[C@H]2N(*)CCCC2)CC1

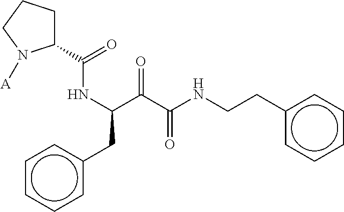

c1ccc(CCNC(═O)C(═O)[C@H](NC(═O)[C@@H]2N(*)CCC2)Cc2ccccc2)cc1

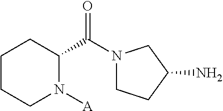

N[C@H]1CN(C(═O)[C@@H]2N(*)CCCC2)CC1

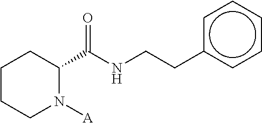

c1ccc(CCNC(═O)[C@@H]2N(*)CCCC2)cc1

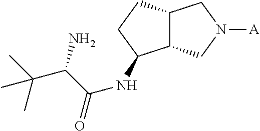

N[C@H](C(═O)N[C@H]1CC[C@@H]2CN(*)C[C@H]12)C(C)(C)C

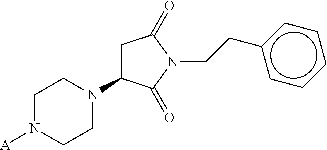

c1ccc(CCN2C(═O)C[C@H](N3CCN(*)CC3)C2═O)cc1

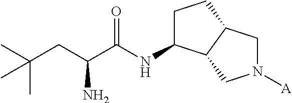

N[C@H](C(—O)N[C@H]1CC[C@@H]2CN(*)C[C@H]12)CC(C)(C)C

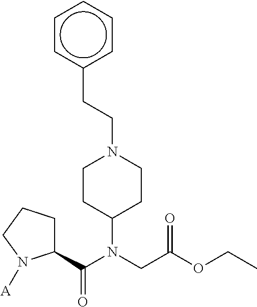

c1ccc(CCN2CCC(N(C(═O)[C@H]3N(*)CCC3)CC(═O)OCC)CC2)cc1

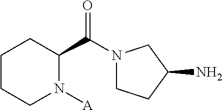

N[C@@H]1CN(C(═O)[C@H]2N(*)CCCC2)CC1

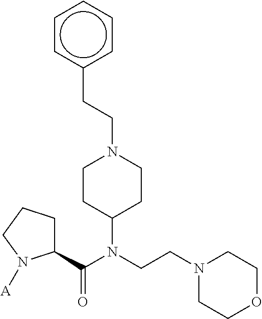

c1ccc(CCN2CCC(N(C(═O)[C@H]3N(*)CCC3)CCN3CCOCC3)CC2)cc1

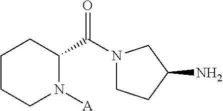

N[C@@H]1CN(C(═O)[C@@H]2N(*)CCCC2)CC1

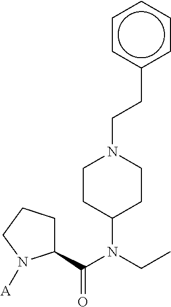

c1ccc(CCN2CCC(N(C(═O)[C@H]3N(*)CCC3)CC)CC2)cc1

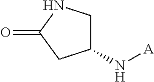

N1C(—O)C[C@@H](N*)C1

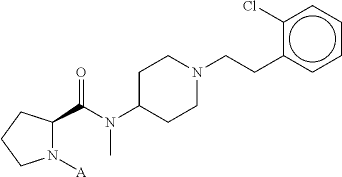

c1ccc(CCN2CCC(N(C(═O)[C@H]3N(*)CCC3)C)CC2)c(C)c1

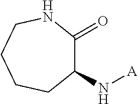

N1C(═O)[C@@H](N*)CCCC1

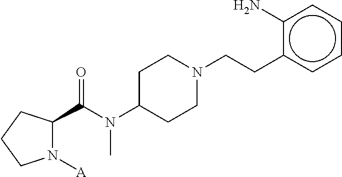

c1ccc(CCN2CCC(N(C(═O)[C@H]3N(*)CCC3)C)CC2)c(N)c1

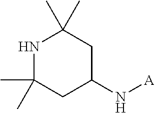

N1C(C)(C)CC(N*)CC1(C)C

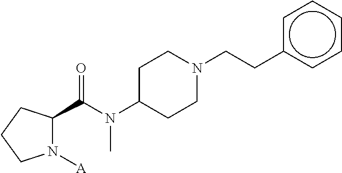

c1ccc(CCN2CCC(N(C(═O)[C@H]3N(*)CCC3)C)CC2)cc1

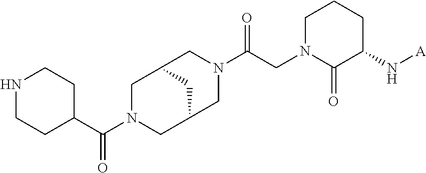

N1CCC(C(—O)N2C[C@H]3CN(C(—O)CN4C(—O)[C@@H](N*)CCC4)C[C@@H]
(C2)C3)CC1

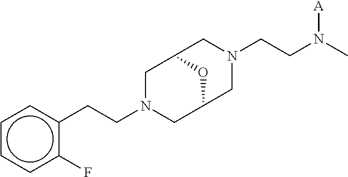

c1ccc(CCN2C[C@@H]3O[C@H](C2)CN(CCN(C)*)C3)c(F)c1

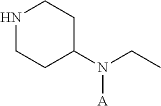

N1CCC(N(CC)*)CC1

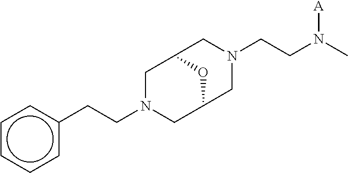

c1ccc(CCN2C[C@@H]3O[C@H](C2)CN(CCN(C)*)C3)cc1

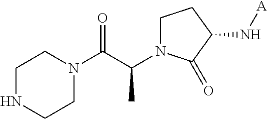

N1CCN(C(═O)[C@@H](N2C(═O)[C@@H](N*)CC2)C)CC1

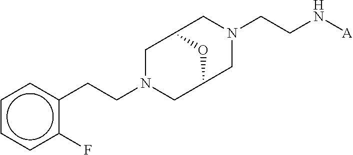

c1ccc(CCN2C[C@@H]3O[C@H](C2)CN(CCN*)C3)c(F)c1

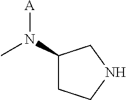

N1CC[C@@H](N(C)*)C1

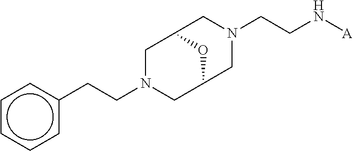

c1ccc(CCN2C[C@@H]3O[C@H](C2)CN(CCN*)C3)cc1

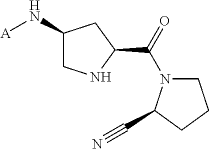

N1C[C@@H](N*)C[C@H]1C(═O)N1CCC[C@H]1C#N

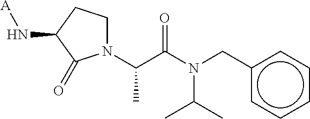

c1ccc(CN(C(═O)[C@@H](N2C(═O)[C@@H](N*)CC2)C)C(C)C)cc1

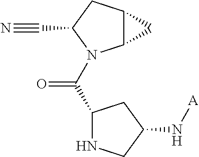

N1C[C@@H](N*)C[C@H]1C(═O)N1[C@H](C#N)C[C@@H]2C[C@H]12

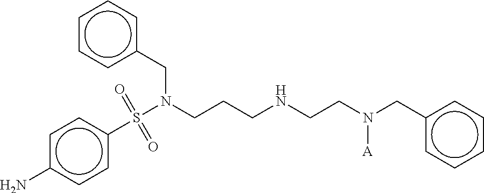

c1ccc(CN(CCCNCCN(Cc2ccccc2)*)S(═O)(═O)c2ccc(N)cc2)cc1

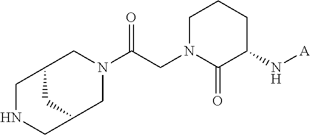

N1C[C@H]2CN(C(—O)CN3C(—O)[C@@H](N*)CCC3)C[C@@H](C1)C2

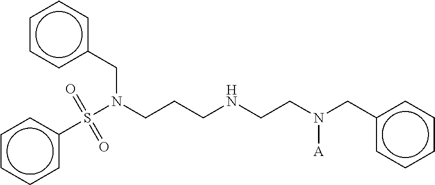

c1ccc(CN(CCCNCCN(Cc2ccccc2)*)S(═O)(═O)c2ccccc2)cc1

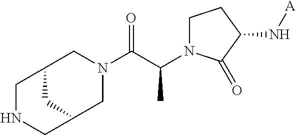

N1C[C@H]2CN(C(═O)[C@@H](N3C(═O)[C@@H](N*)CC3)C)C[C@@H](C1)C2

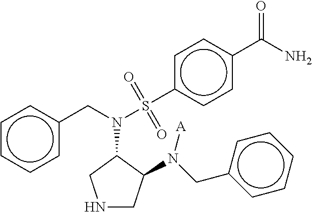

c1ccc(CN([C@H]2CNC[C@@H]2N(Cc2ccccc2)*)S(═O)(═O)c2ccc(C(═O)N)cc2)cc1

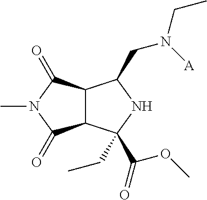

N1[C@H](CN(CC)*)[C@@H]2C(═O)N(C)C(═O)[C@@H]2[C@@]1(C(═O)OC)CC

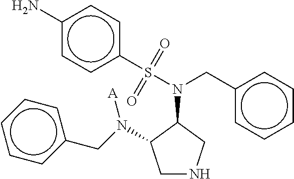

c1ccc(CN([C@H]2CNC[C@@H]2N(Cc2ccccc2)*)S(═O)(═O)c2ccc(N)cc2)cc1

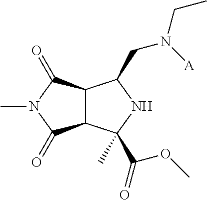

N1[C@H](CN(CC)*)[C@@H]2C(—O)N(C)C(—O)[C@@H]2[C@@]1(C(—O)OC)C

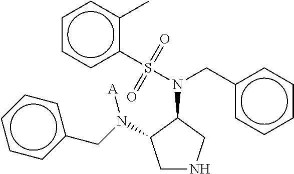

c1ccc(CN([C@H]2CNC[C@@H]2N(Cc2ccccc2)*)S(═O)(═O)c2ccccc2C)cc1

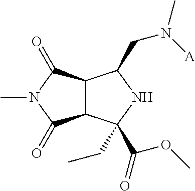

N1[C@H](CN(C)*)[C@@H]2C(═O)N(C)C(═O)[C@@H]2[C@@]1(C(═O)OC)CC

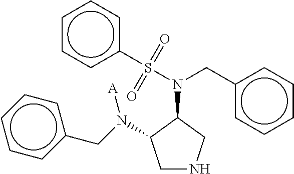

c1ccc(CN([C@H]2CNC[C@@H]2N(Cc2ccccc2)*)S(═O)(═O)c2ccccc2)cc1

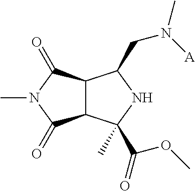

N1[C@H](CN(C)*)[C@@H]2C(═O)N(C)C(═O)[C@@H]2[C@@]1(C(═O)OC)C

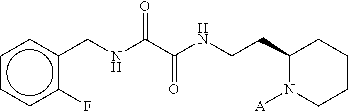

c1ccc(CNC(═O)C(═O)NCC[C@@H]2N(*)CCCC2)c(F)c1

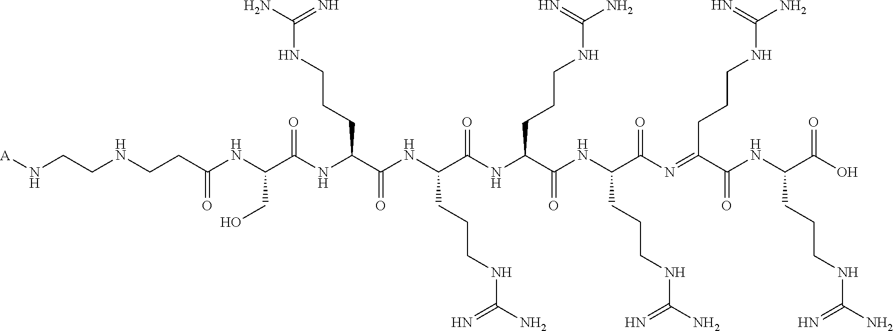

N—C(N)NCCC/C(—N\C(—O)[C@@H](NC(—O)[C@@H](NC(—O)[C@@H](NC(—O)
[C@@H](NC(—O)[C@@H](NC(—O)CCNCCN*)CO)CCCNC(—N)N)CCCNC(—N)N)
CCCNC(—N)N)/C(—O)N[C@H](C(—O)O)CCCNC(—N)N

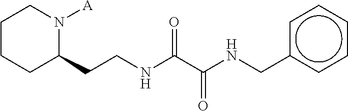

c1ccc(CNC(—O)C(—O)NCC[C@@H]2N(*)CCCC2)cc1

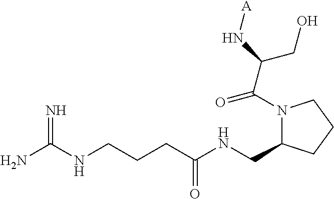

N═C(N)NCCCC(═O)NC[C@H]1N(C(═O)[C@@H](N*)CO)CCC1

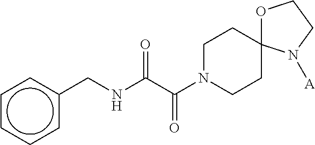

c1ccc(CNC(═O)C(═O)N2CCC3(OCCN3*)CC2)cc1

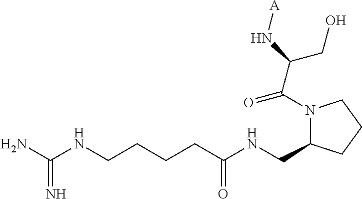

N═C(N)NCCCCC(═O)NC[C@H]1N(C(═O)[C@@H](N*)CO)CCC1

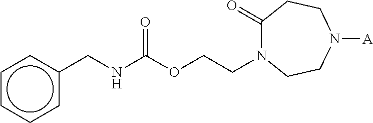

c1ccc(CNC(═O)OCCN2C(═O)CCN(*)CC2)cc1

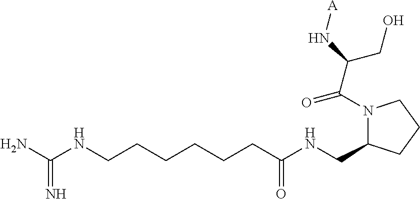

N—C(N)NCCCCCCC(—O)NC[C@H]1N(C(—O)[C@@H](N*)CO)CCC1

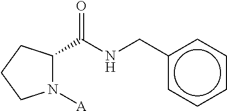

c1ccc(CNC(═O)[C@@H]2N(*)CCC2)cc1

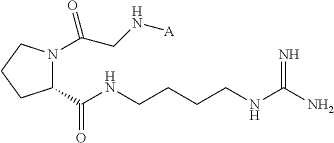

N═C(N)NCCCCNC(═O)[C@H]1N(C(═O)CN*)CCC1

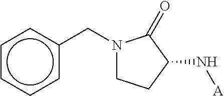

c1ccc(CN2C(═O)[C@H](*)CC2)cc1

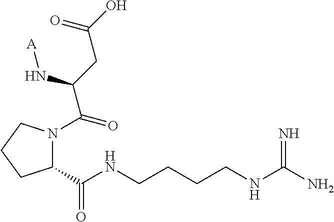

N═C(N)NCCCCNC(═O)[C@H]1N(C(═O)[C@@H](N*)CC(═O)O)CCC1

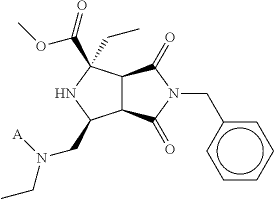

c1ccc(CN2C(═O)[C@@H]3[C@@](C(═O)OC)(CC)N[C@H](CN(CC)*)[C@@H]3C2═O)cc1

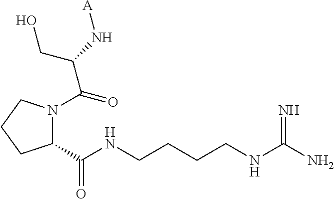

N—C(N)NCCCCNC(—O)[C@H]1N(C(—O)[C@@H](N*)CO)CCC1

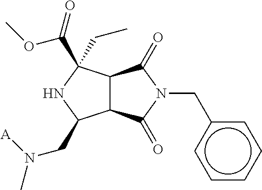

c1ccc(CN2C(═O)[C@@H]3[C@@](C(═O)OC)(CC)N[C@H](CN(C)*)[C@@H]3C2═O)cc1

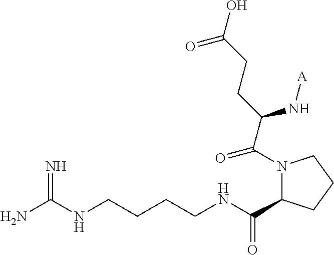

N═C(N)NCCCCNC(═O)[C@H]1N(C(═O)[C@H](N*)CCC(═O)O)CCC1

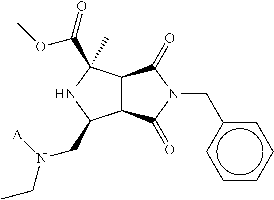

c1ccc(CN2C(═O)[C@@H]3[C@@](C(═O)OC)(C)N[C@H](CN(CC)*)[C@@H]3C2═O)cc1

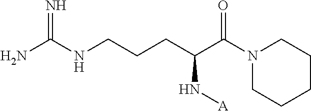

N═C(N)NCCC[C@@H](C(═O)N1CCCCC1)N*

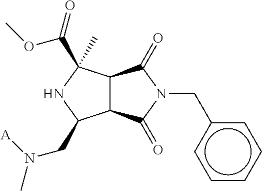

c1ccc(CN2C(═O)[C@@H]3[C@@](C(═O)OC)(C)N[C@H](CN(C)*)[C@@H]3C2═O)cc1

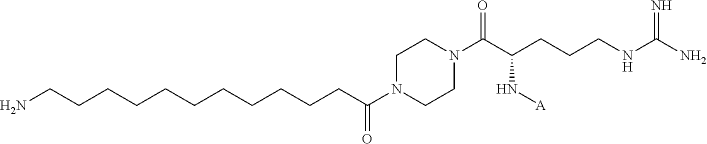

N—C(N)NCCC[C@@H](C(—O)N1CCN(C(—O)CCCCCCCCCCCN)CC1)N*

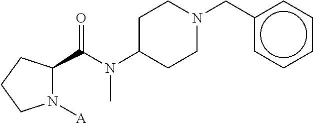

c1ccc(CN2CCC(N(C(═O)[C@H]3N(*)CCC3)C)CC2)cc1

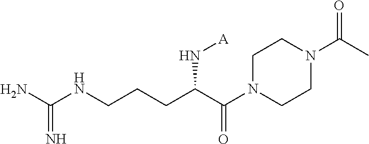

N═C(N)NCCC[C@@H](C(═O)N1CCN(C(═O)C)CC1)N*

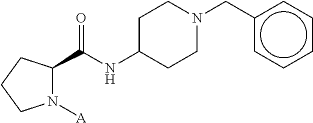

c1ccc(CN2CCC(NC(═O)[C@H]3N(*)CCC3)CC2)cc1

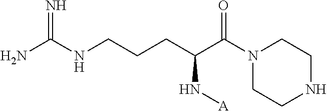

N═C(N)NCCC[C@@H](C(═O)N1CCNCC1)N*

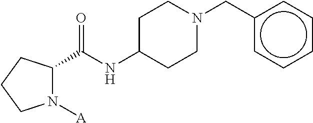

c1ccc(CN2CCC(NC(═O)[C@@H]3N(*)CCC3)CC2)cc1

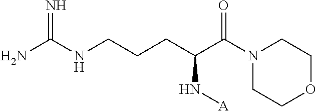

N—C(N)NCCC[C@@H](C(—O)N1CCOCC1)N*

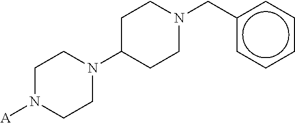

c1ccc(CN2CCC(N3CCN(*)CC3)CC2)cc1

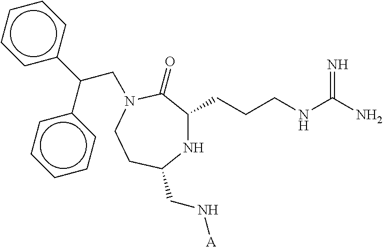

N═C(N)NCCC[C@H]1C(═O)N(CC(c2ccccc2)c2ccccc2)CC[C@@H](CN*)N1

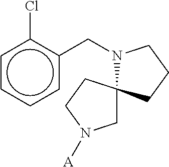

c1ccc(CN2CCC[C@@]32CN(*)CC3)c(C1)c1

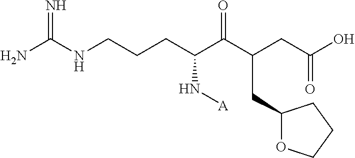

N═C(N)NCCC[C@H](C(═O)N(CC(═O)O)C[C@@H]1OCCC1)N*

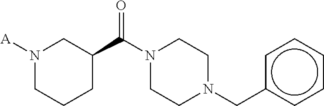

c1ccc(CN2CCN(C(═O)[C@@H]3N(*)CCC3)CC2)cc1

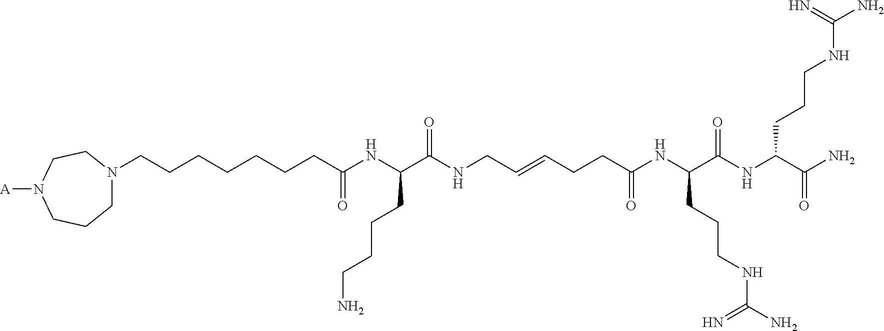

N—C(N)NCCC[C@H](C(—O)N)NC(—O)[C@H](NC(—O)CC/C—C/CNC(—O)[C@H]
(NC(—O)CCCCCCCN1CCN(*)CCC1)CCCCN)CCCNC(—N)N

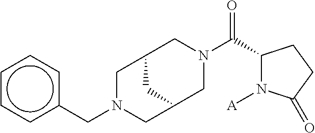

c1ccc(CN2C[C@H]3CN(C(═O)[C@H]4N(*)C(═O)CC4)C[C@@H](C2)C3)cc1

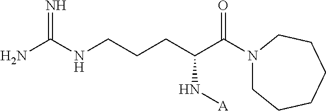

N═C(N)NCCC[C@H](C(═O)N1CCCCCC1)N*

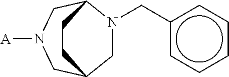

c1ccc(CN2C[C@@H]3CN(*)C[C@H]2CC3)cc1

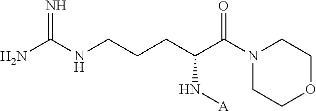

N═C(N)NCCC[C@H](C(═O)N1CCOCC1)N*

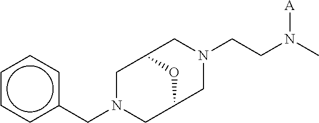

c1ccc(CN2C[C@@H]3O[C@H](C2)CN(CCN(C)*)C3)cc1

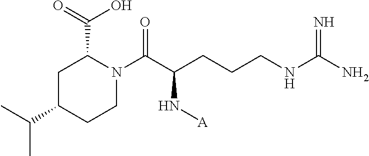

N—C(N)NCCC[C@H](C(—O)N1CC[C@H](C(C)C[C@@H]1C(—O)O)N*

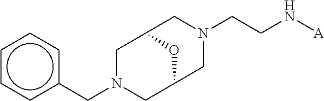

c1ccc(CN2C[C@@H]3O[C@H](C2)CN(CCN*)C3)cc1

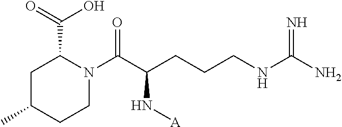

N═C(N)NCCC[C@H](C(═O)N1CC[C@H](C)C[C@@H]1C(═O)O)N*

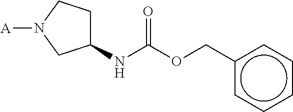

c1ccc(COC(═O)N[C@H]2CN(*)CC2)cc1

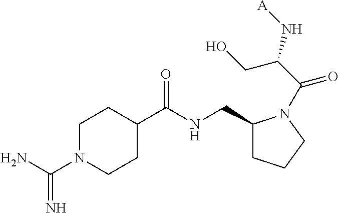

N═C(N)N1CCC(C(═O)NC[C@H]2N(C(═O)[C@@H](N*)CO)CCC2)CC1

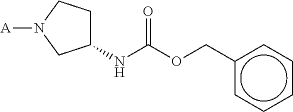

c1ccc(COC(═O)N[C@@H]2CN(*)CC2)cc1

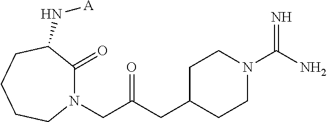

N—C(N)N1CCC(CC(—O)CN2C(—O)[C@@H](N*)CCCC2)CC1

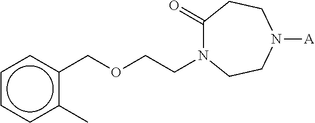

c1ccc(COCCN2C(═O)CCN(*)CC2)c(C)c1

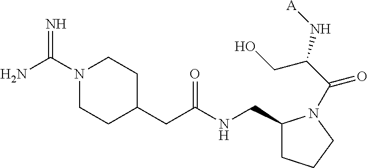

N═C(N)N1CCC(CC(═O)NC[C@H]2N(C(═O)[C@@H](N*)CO)CCC2)CC1

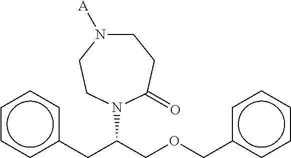

c1ccc(COC[C@@H](N2C(═O)CCN(*)CC2)Cc2ccccc2)cc1

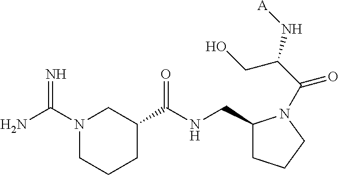

N═C(N)N1CCC[C@@H](C(═O)NC[C@H]2N(C(═O)[C@@H](N*)CO)CCC2)C1

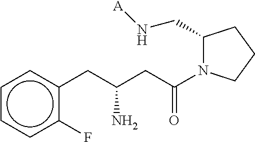

c1ccc(C[C@@H](N)CC(═O)N2CCC[C@H]2CN*)c(F)c1

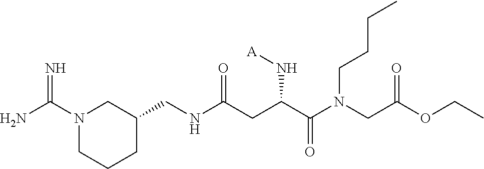

N—C(N)N1CCC[C@@H](CNC(—O)C[C@@H](C(—O)N(CC(—O)OCC)CCCC)N*)C1

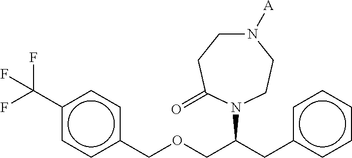

c1ccc(C[C@H](N2C(═O)CCN(*)CC2)COCc2ccc(C(F)(F)F)cc2)cc1

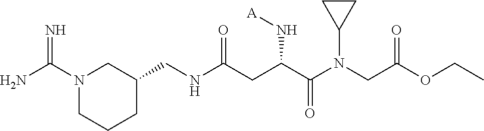

N═C(N)N1CCC[C@@H](CNC(═O)C[C@@H](C(═O)N(CC(═O)OCC)C2CC2)N*)C1

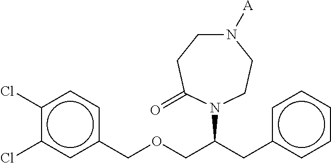

c1ccc(C[C@H](N2C(═O)CCN(*)CC2)COCc2ccc(C1)c(C1)c2)cc1

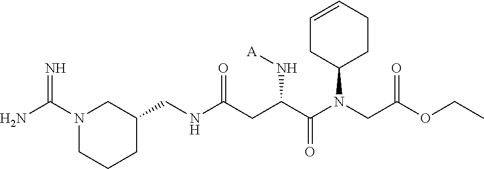

N═C(N)N1CCC[C@@H](CNC(═O)C[C@@H](C(═O)N(CC(═O)OCC)[C@H]
2CC═CCC2)N*)C1

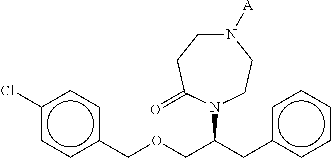

c1ccc(C[C@H](N2C(═O)CCN(*)CC2)COCc2ccc(C1)cc2)cc1

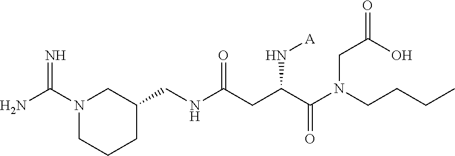

N—C(N)N1CCC[C@@H](CNC(—O)C[C@@H](C(—O)N(CC(—O)O)CCCC)N*)C1

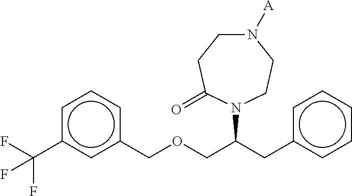

c1ccc(C[C@H](N2C(═O)CCN(*)CC2)COCc2cccc(C(F)(F)F)c2)cc1

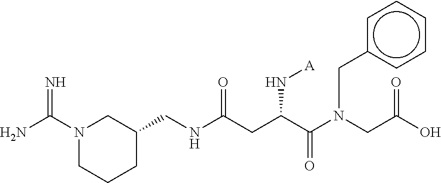

N═C(N)N1CCC[C@@H](CNC(═O)C[C@@H](C(═O)N(CC(═O)O)Cc2ccccc2)N*)C1

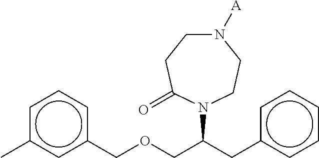

c1ccc(C[C@H](N2C(═O)CCN(*)CC2)COCc2cccc(C)c2)cc1

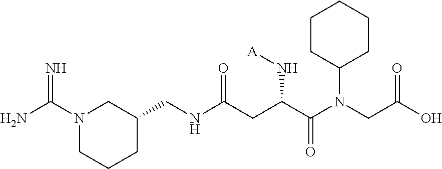

N═C(N)N1CCC[C@@H](CNC(═O)C[C@@H](C(═O)N(CC(═O)O)C2CCCCC2)N*)C1

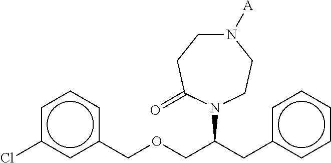

c1ccc(C[C@H](N2C(═O)CCN(*)CC2)COCc2cccc(C)c2)cc1

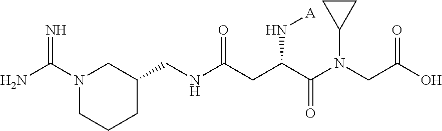

N—C(N)N1CCC[C@@H](CNC(—O)C[C@@H](C(—O)N(CC(—O)O)C2CC2)N*)C1

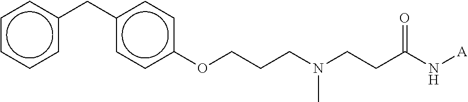

c1ccc(Cc2ccc(OCCCCN(C)CCC(═O)N*)cc2)cc1

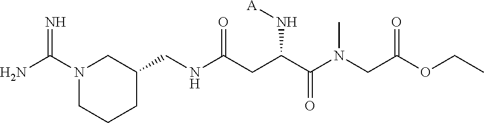

N═C(N)N1CCC[C@@H](CNC(═O)C[C@@H](C(═O)N(C)CC(═O)OCC)N*)C1

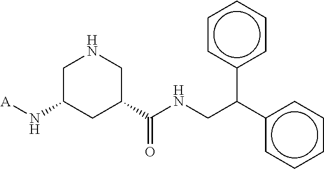

c1ccc(C(c2ccccc2)CNC(═O)[C@H]2CNC[C@@H](N*)C2)cc1

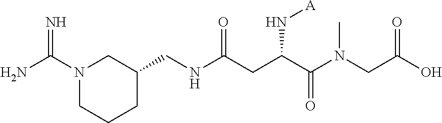

N═C(N)N1CCC[C@@H](CNC(═O)C[C@@H](C(═O)N(C)CC(═O)O)N*)C1

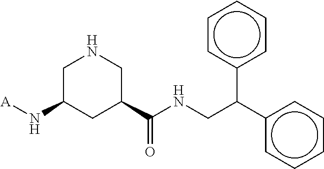

c1ccc(C(c2ccccc2)CNC(═O)[C@@H]2CNC[C@H](N*)C2)cc1

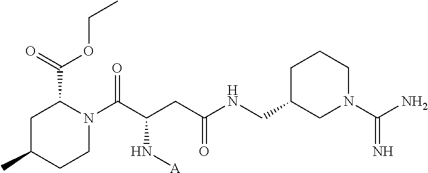

N—C(N)N1CCC[C@@H](CNC(—O)C[C@@H](C(—O)N2CC[C@@H](C)C[C@@H]
2C(—O)OCC)N*)C1

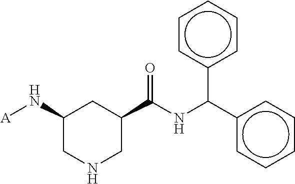

c1ccc(C(c2ccccc2)NC(═O)[C@H]2CNC[C@@H](N*)C2)cc1

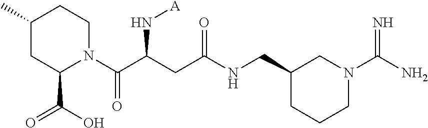

N═C(N)N1CCC[C@@H](CNC(═O)C[C@@H](C(═O)N2CC[C@@H](C)C[C@@H]
2C(═O)O)N*)C1

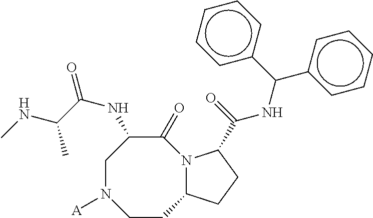

c1ccc(C(c2ccccc2)NC(═O)[C@H]2N3C(═O)[C@@H](NC(═O)[C@@H](NC)C)CN(*)CC[C@H]
3CC2)cc1

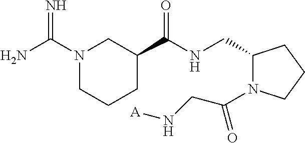

N═C(N)N1CCC[C@H](C(═O)NC[C@H]2N(C(═O)CN*)CCC2)C1

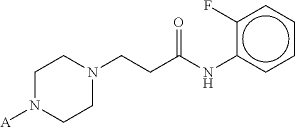

c1ccc(NC(═O)CCN2CCN(*)CC2)c(F)c1

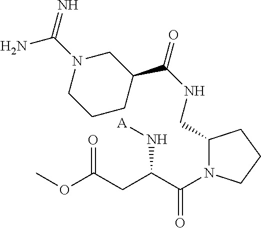

N—C(N)N1CCC[C@H](C(—O)NC[C@H]2N(C(—O)[C@@H](N*)CC(—O)OC)CCC2)C1

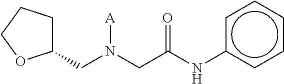

c1ccc(NC(═O)CN(C[C@@H]2OCCC2)*)cc1

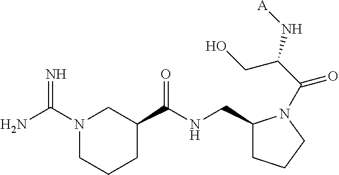

N═C(N)N1CCC[C@H](C(═O)NC[C@H]2N(C(═O)[C@@H](N*)CO)CCC2)C1

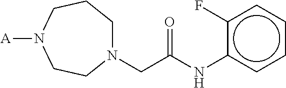

c1ccc(NC(═O)CN2CCN(*)CCC2)c(F)c1

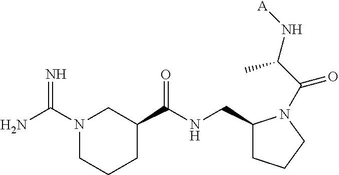

N═C(N)N1CCC[C@H](C(═O)NC[C@H]2N(C(═O)[C@@H](N*)C)CCC2)C1

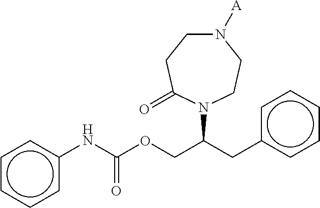

c1ccc(NC(═O)OC[C@@H](N2C(═O)CCN(*)CC2)Cc2ccccc2)cc1

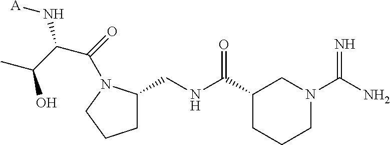

N—C(N)N1CCC[C@H](C(—O)NC[C@H]2N(C(—O)[C@@H](N*)[C@@H](O)C)CCC2)C1

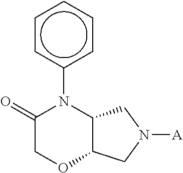

c1ccc(N2C(═O)CO[C@H]3CN(*)C[C@@H]23)cc1

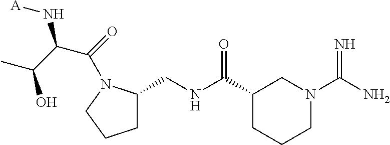

N═C(N)N1CCC[C@H](C(═O)NC[C@H]2N(C(═O)[C@H](N*)[C@@H](O)C)CCC2)C1

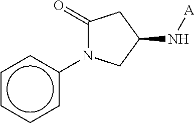

c1ccc(N2C(═O)C[C@@H](N*)C2)cc1

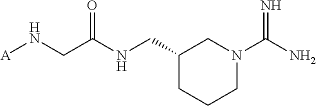

N═C(N)N1CCC[C@H](CNC(═O)CN*)C1

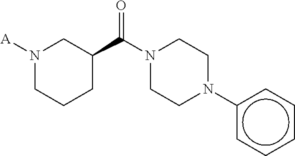

c1ccc(N2CCN(C(═O)[C@@H]3CN(*)CCC3)CC2)cc1

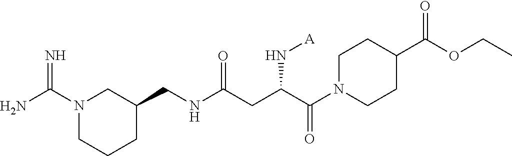

N—C(N)N1CCC[C@H](CNC(—O)C[C@@H](C(—O)N2CCC(C(—O)OCC)CC2)N*)C1

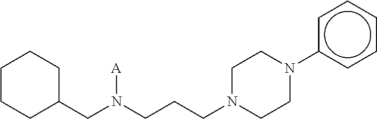

c1ccc(N2CCN(CCCN(CC3CCCCC3)*)CC2)cc1

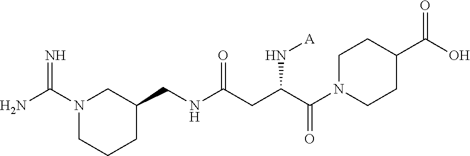

N═C(N)N1CCC[C@H](CNC(═O)C[C@@H](C(═O)N2CCC(C(═O)O)CC2)N*)C1

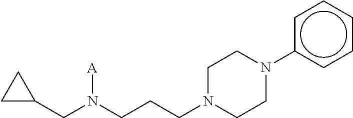

c1ccc(N2CCN(CCCN(CC3CC3)*)CC2)cc1

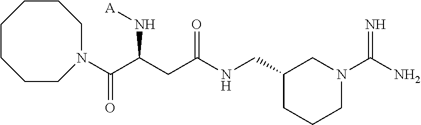

N═C(N)N1CCC[C@H](CNC(═O)C[C@@H](C(═O)N2CCCCCCC2)N*)C1

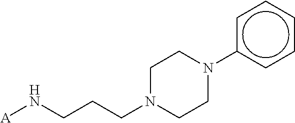

c1ccc(N2CCN(CCCN*)CC2)cc1

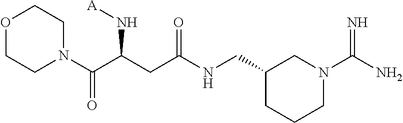

N—C(N)N1CCC[C@H](CNC(—O)C[C@@H](C(—O)N2CCOCC2)N*)C1

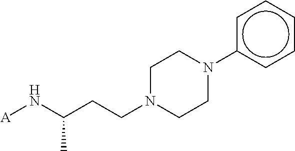

c1ccc(N2CCN(CC[C@@H](N*)C)CC2)cc1

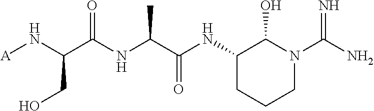

N═C(N)N1CCC[C@H](NC(═O)[C@@H](NC(═O)[C@H](N*)CO)C)[C@@H]1O

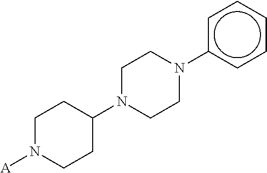

c1ccc(N2CCN(C3CCN(*)CC3)CC2)cc1

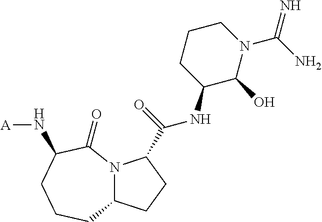

N═C(N)N1CCC[C@H](NC(═O)[C@H]2N3C(═O)[C@H](N*)CCC[C@H]3CC2)[C@@H]1O

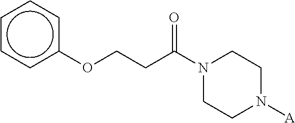

c1ccc(OCCC(═O)N2CCN(*)CC2)cc1

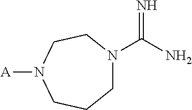

N—C(N)N1CCN(*)CCC1

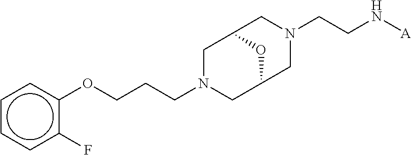

c1ccc(OCCCN2C[C@@H]3O[C@@H](CN(CCN*)C3)C2)c(F)c1

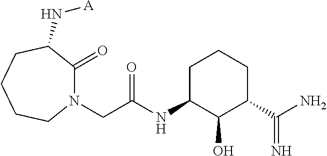

N═C(N)[C@H]1CCC[C@H](NC(═O)CN2C(═O)[C@@H](N*)CCCC2)[C@@H]1O

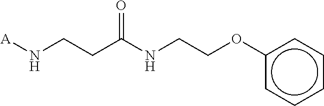

c1ccc(OCCNC(═O)CCN*)cc1

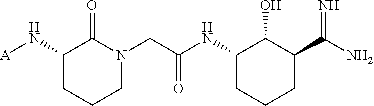

N═C(N)[C@H]1CCC[C@H](NC(═O)CN2C(═O)[C@@H](N*)CCC2)[C@@H]1O

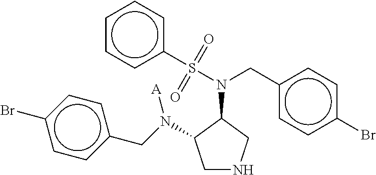

c1ccc(S(═O)(═O)N(Cc2ccc(Br)cc2)[C@H]2CNC[C@@H]2N(Cc2ccc(Br)cc2)*)cc1

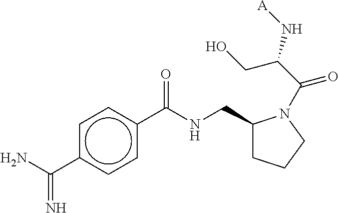

N—C(c1ccc(C(—O)[C@H]2N(C(—O)[C@@H](N*)CO)CCC2)cc1)N

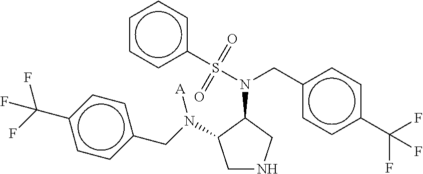

c1ccc(S(═O)(═O)N(Cc2ccc(C(F)(F)F)cc2)[C@H]2CNC[C@@H]2N(Cc2ccc(C(F)(F)F)cc2)*)cc1

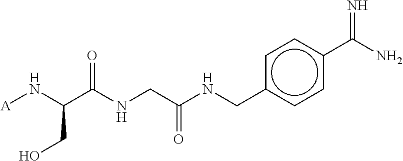

N═C(c1ccc(CNC(═O)CNC(═O)[C@H](N*)CO)cc1)N

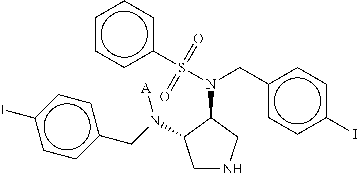

c1ccc(S(═O)(═O)N(Cc2ccc(I)cc2)[C@H]2CNC[C@@H]2N(Cc2ccc(I)cc2)*)cc1

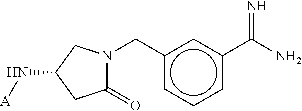

N═C(c1cccc(CN2C(═O)C[C@H](N*)C2)c1)N

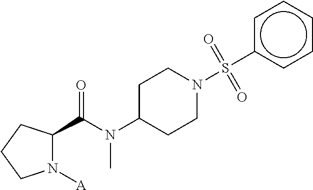

c1ccc(S(═O)(═O)N2CCC(N(C(═O)[C@H]3N(*)CCC3)C)CC2)cc1

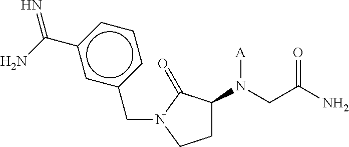

N—C(c1cccc(CN2C(—O)[C@@H](N(CC(—O)N)*)CC2)c1)N

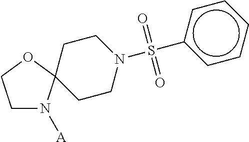

c1ccc(S(═O)(═O)N2CCC3(OCCN3*)CC2)cc1

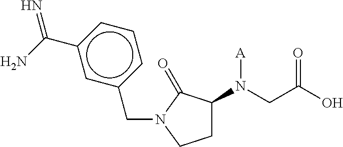

N═C(c1cccc(CN2C(═O)[C@@H](N(CC(═O)O)*)CC2)c1)N

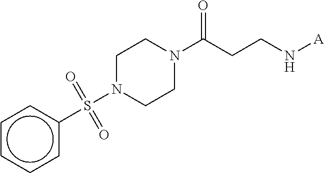

c1ccc(S(═O)(═O)N2CCN(C(═O)CCN*)CC2)cc1

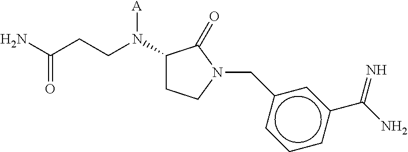

N═C(c1cccc(CN2C(═O)[C@@H](N(CCC(═O)N)*)CC2)c1)N

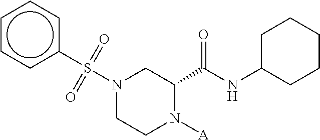

c1ccc(S(═O)(═O)N2CCN(*)[C@@H](C(═O)NC3CCCCC3)C2)cc1

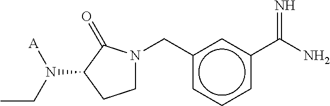

N—C(c1cccc(CN2C(—O)[C@@H](N(CC)*)CC2)c1)N

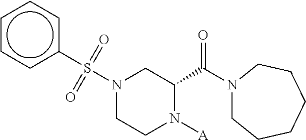

c1ccc(S(═O)(═O)N2CCN(*)[C@@H](C(═O)NC3CCCCCC3)C2)cc1

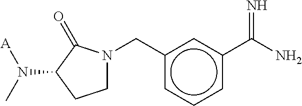

N═C(c1cccc(CN2C(═O)[C@@H](N(C)*)CC2)c1)N

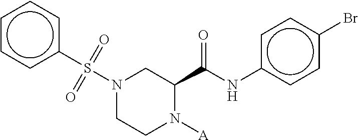

c1ccc(S(═O)(═O)N2CCN(*)[C@H](C(═O)Nc3ccc(Br)cc3)C2)cc1

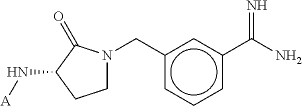

N═C(c1cccc(CN2C(═O)[C@@H](N*)CC2)c1)N

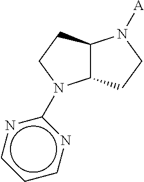

c1nc(N2CC[C@H]3N(*)CC[C@H]23)ncc1

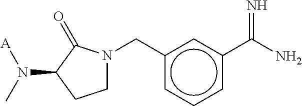

N—C(c1cccc(CN2C(—O)[C@H](N(C)*)CC2)c1)N

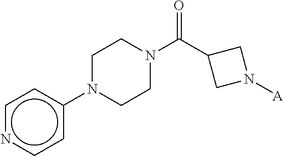

c1nccc(N2CCN(C(═O)C3CN(*)C3)CC2)c1

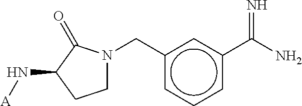

N═C(c1cccc(CN2C(═O)[C@H](N*)CC2)c1)N

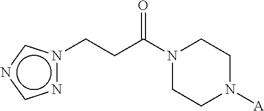

c1ncn(CCC(═O)N2CCN(*)CC2)n1

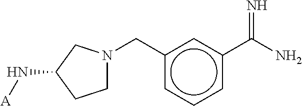

N═C(c1cccc(CN2CC[C@H](N*)C2)c1)N

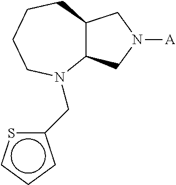

c1sc(CN2CCCC[C@H]3CN(*)C[C@@H]23)cc1

### Additional or Alternative Examples

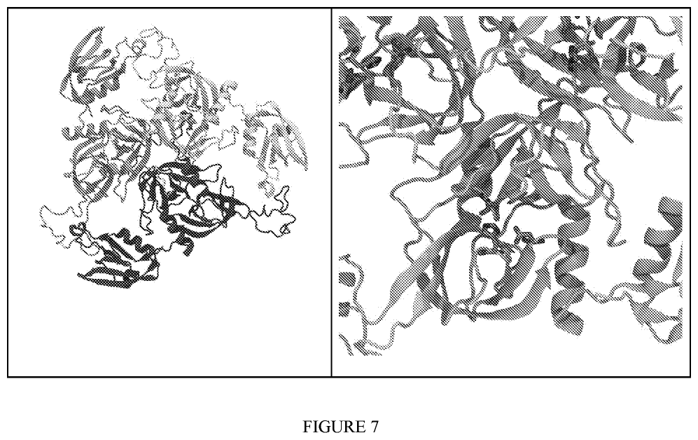

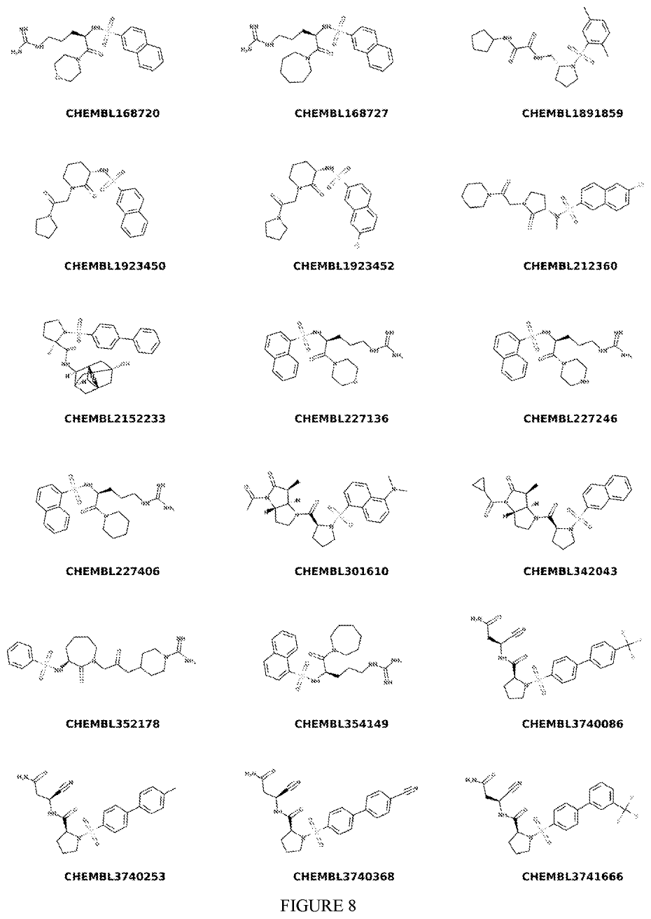

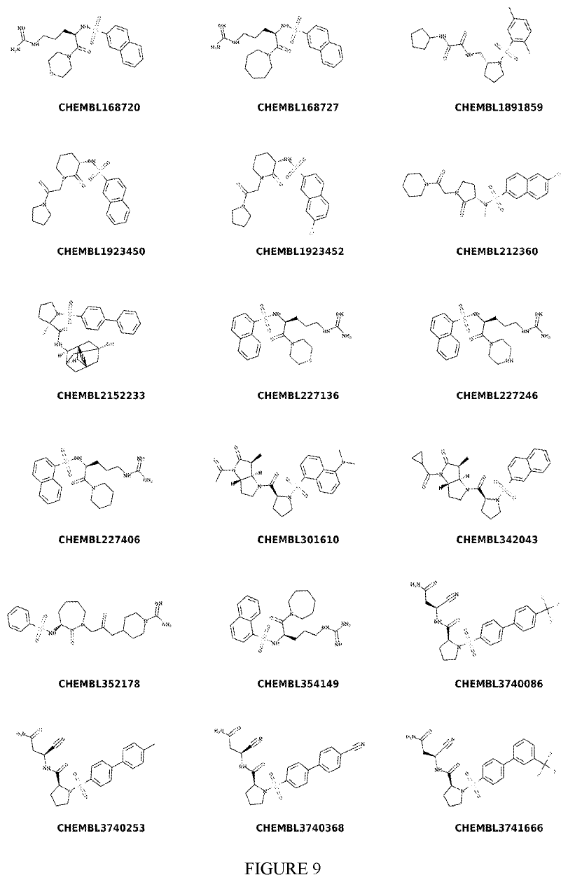

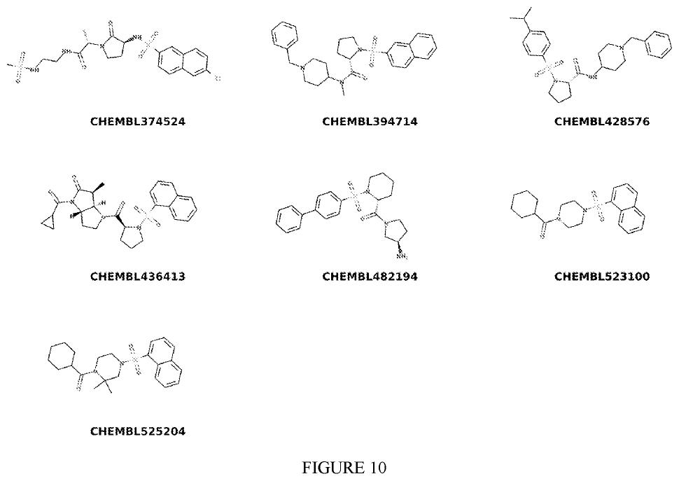

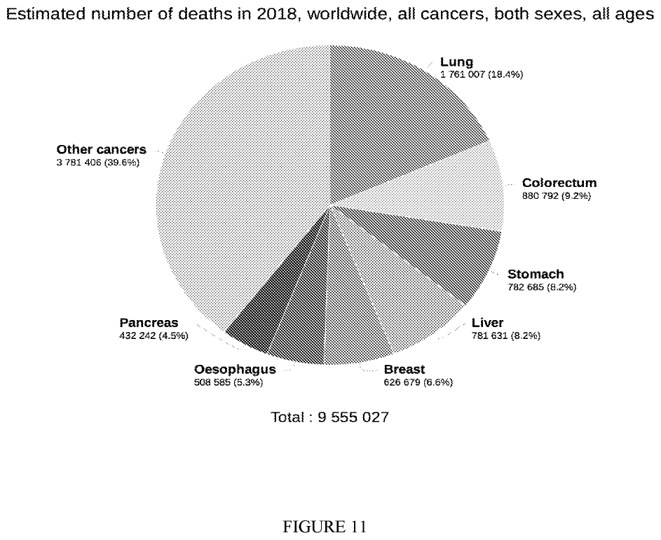

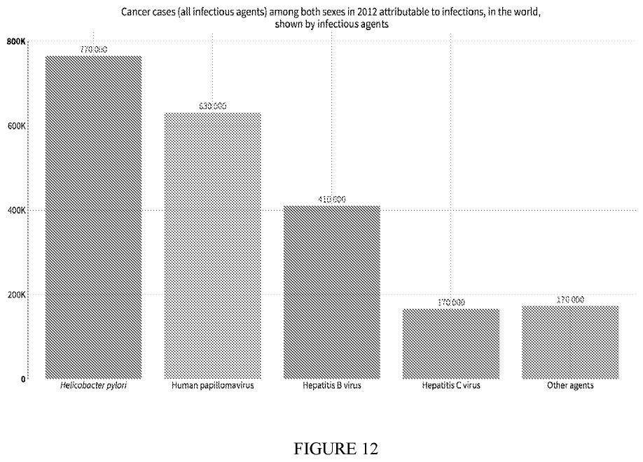

In examples, after the virtual screening (and/or at any suitable time and frequency), the first nine molecules with binding energy less than −9.5 kcal/mol against the HtrA enzymes (however, any suitable criteria can be used, such as any suitable binding energy threshold; etc.) were chosen as the selected candidates (e.g., called “Dataset 1”, FIG. 7). Additionally or alternatively, three additional or alternative molecules with molecular weight higher than 500 Da (however, any suitable criteria can be used, such as any suitable molecular weight threshold; etc.) were also considered in the dataset due to their high energy of binding (e.g., CHEMBL83186, CHEMBL421919, CHEMBL3429004; etc.), but any suitable criteria can additionally or alternatively be used.

TABLE 7
IUPAC nomenclature and canonical SMILES of
specific examples of selected candidates (Dataset 1).

| Compound | | | |
|---|---|---|---|
| # | Molecule | Canonical SMILES | Name |
| 1 | CHEMBL1515370 | Cc1ccc(cc1)S(═O)(═O)N1CCCC | N′-benzyl-N-[2-[(2R)-1-(4- |
| | (binding energy = −9.6 | [C@@H]1CCNC(═O)C(═O)NCc1ccccc1 | methylphenyl)sulfonylpiperidin-2- |
| | kcal/mol) | | yl]ethyl]oxamide |
| 2 | CHEMBL1868353 | NC(═O)C1CCN(CC1)C(═O)CN | 1-[2-[methyl(naphthalen-2- |
| | (binding energy = −9.6 | (S(═O)(═O)c1ccc2c(c1)cccc2)C | ylsulfonyl)amino]acetyl]piperidine-4- |
| | kcal/mol) | | carboxamide |
| 3 | CHEMBL1880232 | Cc1ccc(cc1)S(═O)(═O)N1CCC2 | 8-(4-methylphenyl)sulfonyl-4-(2,4,6- |
| | (binding energy = −9.6 | (CC1)OCCN2S(═O)(═O) | trimethylphenyl)sulfonyl-1-oxa-4,8- |
| | kcal/mol) | c1c(C)cc(cc1C)C | diazaspiro[4.5]decane |
| 4 | CHEMBL2159481 | Fc1cc(ccc1F)S(═O)(═O)N[C@H] | N-{1-[2-(2-Biphenylyloxy)ethyl]-3- |
| | (binding energy = −9.6 | 1CCN(C1)CCOc1ccccc1c1ccccc1 | pyrrolidinyl}-3,4- |
| | kcal/mol) | | difluorobenzenesulfonamide |
| 5 | CHEMBL2172063 | OC(═O)[C@H]1CCCN(C1)S(═O) | 1-(2-Anthrylsulfonyl)-3- |
| | (binding energy = −9.7 | (═O)c1ccc2c(c1)cc1c(c2)cccc1 | piperidinecarboxylic acid |
| | kcal/mol) | | |
| 6 | CHEMBL342904 | O═C([C@H]1CCCN(C1)C(═[NH2])N) | (3S)-1-Carbamimidoyl-N-({(2S)-1- |
| | (binding energy = −9.7 | NC[C@@H]1CCCN1C(═O) | [N-(2-naphthylsulfonyl)glycyl]-2- |
| | kcal/mol) | CNS(═O)(═O)c1ccc2c(c1)cccc2 | pyrrolidinyl}methyl)-3- |
| | | | piperidinecarboxamide |
| 7 | CHEMBL421919 | O═C([C@H]1CCCN(C1)C | (3S)-1-Carbamimidoyl-N-({(2S)-1- |
| | (binding energy = −10.2 | (=[NH2])N)NC[C@@H]1CCCN1C | [N-(2-naphthylsulfonyl)-L-alanyl]-2- |
| | kcal/mol) | (═O)[C@@H](NS | pyrrolidinyl}methyl)-3 - |
| | | (═O)(═O)c1ccc2c(c1)cccc2)C | piperidinecarboxamide |
| 8 | CHEMBL489852 | O═C(N1CCN(C[C@H]1C(F)(F)F) | Cyclohexyl[4-(1-naplithylsulfonyl)- |
| | (binding energy = −9.7 | S(═O)(═O)c1cccc2c1cccc2)C1CCCCC1 | 2-(trifluoromethyl)-1- |
| | kcal/mol) | | piperazinyl]methanone |

### Additional or Alternative Examples

In examples, after the virtual screening (and/or at any suitable time and frequency), the first nine molecules with binding energy less than −9.5 kcal/mol against the HtrA enzymes (however, any suitable criteria can be used, such as any suitable binding energy threshold; etc.) were chosen as the selected candidates (e.g., called “Dataset 1”, FIG. 7). Additionally or alternatively, three additional or alternative molecules with molecular weight higher than 500 Da (however, any suitable criteria can be used, such as any suitable molecular weight threshold; etc.) were also considered in the dataset due to their high energy of binding (e.g., CHEMBL83186, CHEMBL421919, CHEMBL342904; etc.), but any suitable criteria can additionally or alternatively be used.

TABLE 7
IUPAC nomenclature and canonical SMILES of
specific examples of selected candidates (Dataset 1).

| Compound | | | |
|---|---|---|---|
| # | Molecule | Canonical SMILES | Name |
| 1 | CHEMBL1515370 | Cc1ccc(cc1)S(═O)(═O)N1CCCC | N′-benzyl-N-[2-[(2R)-1-(4- |
| | (binding energy = −9.6 | [C@@H]1CCNC(═O)C(═O)NCc1ccccc1 | methylphenyl)sulfonylpiperidin-2 - |
| | kcal/mol) | | yl]ethyl]oxamide |
| 2 | CHEMBL1868353 | NC(═O)C1CCN(CC1)C(═O)CN | 1-[2-[methyl(naphthalen-2- |
| | (binding energy = −9.6 | (S(═O)(═O)c1ccc2c(c1)cccc2)C | ylsulfonyl)amino]acetyl]piperidine-4- |
| | kcal/mol) | | carboxamide |
| 3 | CHEMBL1880232 | Cc1ccc(cc1)S(═O)(═O)N1CCC2 | 8-(4-methylphenyl)sulfonyl-4-(2,4,6- |
| | (binding energy = −9.6 | (CC1)OCCN2S(═O)(═O) | trimethylphenyl)sulfonyl-1-oxa-4,8- |
| | kcal/mol) | c1c(C)cc(cc1C)C | diazaspiro[4.5]decane |
| 4 | CHEMBL2159481 | Fc1cc(ccc1F)S(═O)(═O)N[C@H] | N-{1-[2-(2-Biphenylyloxy)ethyl]-3- |
| | (binding energy = −9.6 | 1CCN(C1)CCOc1ccccc1c1ccccc1 | pyrrolidinyl}-3,4- |
| | kcal/mol) | | difluorobenzenesulfonamide |
| 5 | CHEMBL2172063 | OC(═O)[C@H]1CCCN(C1)S(═O) | 1-(2-Anthrylsulfonyl)-3- |
| | (binding energy = −9.7 | (═O)c1ccc2c(c1)cc1c(c2)cccc1 | piperidinecarboxylic acid |
| | kcal/mol) | | |
| 6 | CHEMBL342904 | O═C([C@H]1CCCN(C1)C(═[NH2])N) | (3S)-1-Carbamimidoyl-N-({(2S)-1- |
| | (binding energy = −9.7 | NC[C@@H]1CCCN1C(═O) | [N-(2-naphthylsulfonyl)glycyl]-2- |
| | kcal/mol) | CNS(═O)(═O)c1ccc2c(c1)cccc2 | pyrrolidinyl}methyl)-3- |
| | | | piperidinecarboxamide |
| 7 | CHEMBL421919 | O═C([C@H]1CCCN(C1)C(=[N | (3S)-1-Carbamimidoyl-N-({(2S)-1- |
| | (binding energy = −10.2 | H2])N)NC[C@@H]1CCCN1C | [N-(2-naphthylsulfonyl)-L-alanyl]-2- |
| | kcal/mol) | (═O)[C@@H](NS | pyrrolidinyl}methyl)-3 - |
| | | (═O)(═O)c1ccc2c(c1)cccc2)C | piperidinecarboxamide |
| 8 | CHEMBL489852 | O═C(N1CCN(C[C@H]1C(F)(F)F) | Cyclohexyl[4-(1-naplithylsulfonyl)- |
| | (binding energy = −9.7 | S(═O)(═O)c1cccc2c1cccc2)C1CCCCC1 | 2-(trifluoromethyl)-1- |
| | kcal/mol) | | piperazinyl]methanone |

TABLE 8
Specific examples of ADME properties of the selected candidates (Dataset 1)
obtained in SwissADME. In specific examples, Compounds 2, 6 and 7 appeared as a good drug
candidates, as they are predicted do not inhibit cytochromes P450 isoforms.

| Compound | 1 | 2 | 3 | 4 | 5 | 6 | 7 | 8 | 9 |
|---|---|---|---|---|---|---|---|---|---|
| MW | 443.56 | 389.47 | 478.62 | 458.52 | 369.43 | 501.62 | 515.65 | 454.51 | 592.75 |
| #H-bond | 5 | 5 | 7 | 7 | 5 | 5 | 5 | 7 | 8 |
| acceptors | | | | | | | | | |
| #H-bond | 2 | 1 | 0 | 1 | 1 | 4 | 4 | 0 | 4 |
| donors | | | | | | | | | |
| TPSA | 103.96 | 109.16 | 100.75 | 67.02 | 83.06 | 159.24 | 159.24 | 66.07 | 176.51 |
| Consensus | 2.62 | 1.35 | 3.23 | 4.11 | 2.94 | −0.27 | 0.23 | 4.01 | 1.64 |
| Log P | | | | | | | | | |
| Ali | Moderately | Soluble | Moderately | Moderately | Moderately | Soluble | Moderately | Moderately | Moderately |
| Class | soluble | | soluble | soluble | soluble | | soluble | soluble | soluble |
| GI | High | High | High | High | High | Low | Low | High | Low |
| absorption | | | | | | | | | |
| BBB | No | No | No | No | No | No | No | No | No |
| permeant | | | | | | | | | |
| CYP1A2 | No | No | No | No | Yes | No | No | No | No |
| inhibitor | | | | | | | | | |
| CYP2C19 | Yes | No | Yes | Yes | No | No | No | Yes | No |
| inhibitor | | | | | | | | | |
| CYP2C9 | Yes | No | Yes | Yes | Yes | No | No | Yes | No |
| inhibitor | | | | | | | | | |
| CYP2D6 | Yes | No | No | Yes | No | No | No | Yes | No |
| inhibitor | | | | | | | | | |
| CYP3A4 | Yes | No | Yes | Yes | Yes | No | No | Yes | Yes |
| inhibitor | | | | | | | | | |

In examples, in a second filter (and/or any suitable filter able to be applied at any suitable time and frequency; etc.), molecules having a binding energy higher than −9.4 kcal/mol and less than −8.9 kcal/mol (e.g., which can be an improvement over the reported compound) (however, any suitable criteria can be used, such as any suitable binding energy threshold; etc.) were chosen as the Dataset 2. In specific examples, two molecules having MW>500 were also included in the dataset 2, as the showed high binding affinity.

TABLE 9
IUPAC nomenclature and canonical SMILES of specific
examples of the selected candidates (Dataset 2).

| Compound | | | |
|---|---|---|---|
| # | Molecule | Canonical SMILES | Name |
| 10 | CHEMBL168720 | NC(═N)NCCC[C@H](C(═O)N1 | N-[5-Carbamimidamido-1-(4 - |
| | | CCOCC1)NS(═O)(═O)c1ccc2c(c1)cccc2 | morpholinyl)-1-oxo-2-pentanyl]- |
| | | | 2-naphthalenesulfonamide |
| 11 | CHEMBL168727 | NC(═N)NCCC[C@H](C(═O) | N-[1-(1-Azepanyl)-5- |
| | | N1CCCCCC1)NS(═O)(═O)c1ccc2c(c1)cccc2 | (carbamimidamido-1-oxo-2- |
| | | | pentanyl]-2- |
| | | | naphthalene sulfonamide |
| 12 | CHEMBL1891859 | Cc1ccc(c(c1)S(═O)(═O)N1CCC | N-Cyclopentyl-N′-({1-[(2,5- |
| | | [C@@H]1CNC(═O)C(═O)NC1CCCC1)C | dimethylphenyl)sulfonyl]-2- |
| | | | pyrrolidinyl}methyl)ethanediamide |
| 13 | CHEMBL1923450 | O═C1N(CCC[C@@H]1NS(═O) | N-{(3S)-2-Oxo-1-[2-oxo-2-(1- |
| | | (═O)c1ccc2c(c1)cccc2)CC(═O)N1CCCC1 | pyrrolidinyl)ethyl]-3- |
| | | | piperidinyl}-2- |
| | | | naphthalene sulfonamide |
| 14 | CHEMBL1923452 | Clc1ccc2c(c1)cc(cc2)S(═O)(═O) | 7-Chloro-N-{(3S)-2-oxo-1-[2- |
| | | N[C@H]1CCCN(C1═O)CC(═O)N1CCCC1 | oxo-2-(1-pyrrolidinyl)ethyl]-3- |
| | | | piperidinyl}-2- |
| | | | naphthalenesulfonamide |
| 15 | CHEMBL212360 | Clc1ccc2c(c1)ccc(c2)S(═O)(═O) | 6-Chloro-N-methyl-N-{(3S)-2- |
| | | N([C@H]1CCN(C1═O)CC(═O)N1CCCCC1)C | oxo-1-[2-oxo-2-(1- |
| | | | piperidinyl)ethyl]-3- |
| | | | pyrrolidinyl}-2- |
| | | | naphthalenesulfonamide |
| 16 | CHEMBL2152233 | O═C([C@@]1(C)CCCN1S(═O) | 1-(4-Biphenylylsulfonyl)-N- |
| | | (═O)c1ccc(cc1)c1ccccc1)N[C@@H]1 | [(2s,5R)-5-hydroxyadamantan-2- |
| | | [C@@H]2C[C@@H]3C[C@H]1C[C@](C2)(C3)O | yl]-2-methyl-D-prolinamide |
| 17 | CHEMBL227136 | NC(═[NH2])NCCC[C@@H](C(═O) | N-[(2S)-5-carbamimidamido-1- |
| | | N1CCOCC1)NS(═O)(═O)c1cccc2c1cccc2 | (morpholin-4-yl)-1-oxopentan-2- |
| | | | yl]naphthalene-1-sulfonamide |
| 18 | CHEMBL227246 | NC(═N)NCCC[C@@H](C(═O) | N-[(2S)-5-carbamimidamido-1- |
| | | N1CCNCC1)NS(═O)(═O)c1cccc2c1cccc2 | oxo-1-(piperazin-1-yl)pentan-2- |
| | | | yl]naphthalene-1-sulfonamide |
| 19 | CHEMBL227406 | NC(═[NH2])NCCC[C@@H](C(═O) | N-[(2S)-5-carbamimidamido-1- |
| | | N1CCCCC1)NS═O)(═O)c1cccc2c1cccc2 | oxo-1-(piperidin-1-yl)pentan-2- |
| | | | yl]naphthalene-1-sulfonamide |
| 20 | CHEMBL301610 | O═C([C@@H]1CCCN1S(═O) | (3S,3aR,6aS)-1-Acetyl-4-{[(2S)- |
| | | (═O)c1cccc2c1cccc2N(C)C)N1CC | 1-{[5-(dimethylamino)-1- |
| | | [C@H]2[C@H]1[C@H](C)C(═O) | naphthyl]sulfonyl}-2- |
| | | N2C(═O)C | pyrrolidinyl]carbonyl}-3- |
| | | | methylhexahydropyrrolo[3,2- |
| | | | b]pyrrol-2(1H)-one |
| 21 | CHEMBL342043 | O═C([C@@H]1CCCN1S(═O)(═O) | (3S,3aR,6aS)-1- |
| | | c1ccc2c(c1)cccc2)N1CC[C@H]2 | (Cyclopropylcarbonyl)-3- |
| | | [C@H]1[C@H](C)C(═O)N2C | methyl-4-{[(2S)-1-(2- |
| | | (═O)C1CC1 | naphthylsulfonyl)-2- |
| | | | pyrrolidinyl]carbonyl}hexahydro |
| | | | pyrrolo[3,2-]pyrrol-2(1H)-one |
| 22 | CHEMBL352178 | O═C(CN1CCCC[C@@H]C1═O) | 4-(2-Oxo-3-{(3S)-2-oxo-3- |
| | | NS(═O)(═O)c1ccccc1)CC1CCN | [(phenylsulfonyl)amino]-1- |
| | | (CC1)C(═[NH2])N | azepanyl}propyl)-1- |
| | | | piperidinecarboximidamide |
| 23 | CHEMBL354149 | NC(═[NH2])NCCC[C@H](C(═O) | N-[1-(1-Azepanyl)-5- |
| | | N1CCCCCC1)NS(═O)(═O)c1cccc2c1cccc2 | carbamimidamido-1-oxo-2- |
| | | | pentanyl]-1- |
| | | | naphthalene sulfonamide |
| 24 | CHEMBL3740086 | N#C[C@@H](NC(═O)[C@@H] | N-[(1S)-3-Amino-1-cyano-3- |
| | | 1CCCN1S(═O)(═O)c1ccc(cc1)c1ccc(cc1)C(F)(F)F) | oxopropyl]-1-{[4′- |
| | | CC(═O)N | (trifluoromethyl)-4- |
| | | | biphenylyl]sulfonyl}-L- |
| | | | prolinamide |
| 25 | CHEMBL3740253 | N#C[C@@H](NC(═O)[C@@H] | N-[(1S)-3-Amino-1-cyano-3- |
| | | 1CCCN1S(═O)(═O)c1ccc(cc1)c1ccc(cc1)C)CC | oxopropyl]-1-[(4′-methyl-4- |
| | | (═O)N | biphenylyl)sulfonyl]-L- |
| | | | prolinamide |
| 26 | CHEMBL3740368 | N#C[C@@H](NC(═O)[C@@H] | N-[(1S)-3-Amino-1-cyano-3- |
| | | 1CCCN1S(═O)(═O)c1ccc(cc1)c1ccc(cc1)C#N)CC | oxopropyl]-1-[(4′-cyano-4- |
| | | (═O)N | biphenylyl)sulfonyl]-L- |
| | | | prolinamide |
| 27 | CHEMBL3741666 | N#C[C@@H](NC(═O)[C@@H] | N-[(1S)-3-Amino-1-cyano-3- |
| | | 1CCCN1S(═O)(═O)c1ccc(cc1)c1cccc(c1)C(F)(F)F) | oxopropyl]-1-{[3′- |
| | | CC(═O)N | (trifluoromethyl)-4- |
| | | | biphenylyl]sulfonyl}-L- |
| | | | prolinamide |
| 28 | CHEMBL374524 | O═C([C@@H](N1CC[C@@H] | (2S)-2-[(3S)-3-{[(6-Chloro-2- |
| | | (C1═O)NS(═O)(═O)c1ccc2c(c1)ccc(c2)Cl)C) | naphthyl)sulfonyl]amino}-2- |
| | | NCCNS(═O)(═O)C | oxo-1-pyrrolidinyl]-N-{2- |
| | | | [(methylsulfonyl)amino]ethyl}pro- |
| | | | panamide |
| 29 | CHEMBL394714 | CN(C(═O)[C@@H]1CCCN1S(═O) | N-(1-Benzyl-4-piperidinyl)-N- |
| | | (═O)c1ccc2c(c1)cccc2)C1CCN | methyl-1-naphthylsulfonyl)- |
| | | (CC1)Cc1ccccc1 | L-prolinamide |
| 30 | CHEMBL428576 | CC(c1ccc(cc1)S(═O)(═O)N1CCC | N-(1-Benzyl-4-piperidinyl)-1- |
| | | [C@H]1C(═O)NC1CCN(CC1)Cc1ccccc1)C | [(4-isopropylphenyl)sulfonyl]-L- |
| | | | prolinamide |
| 31 | CHEMBL436413 | O═C([C@@H]1CCCN1S(═O)(═O) | (3S,3aR,6aS)-1- |
| | | c1cccc2c1cccc2)N1CC[C@H] | (Cyclopropylcarbonyl)-3- |
| | | 2[C@H]1[C@H](C)C(═O)N2C(═O) | methyl-4-{[(2S)-1-(1- |
| | | C1CC1 | naphthylsulfonyl)-2- |
| | | | pyrrolidinyl]carbonyl}hexahydro |
| | | | pyrrolo[3,2-b]pyrrol-2(1H)-one |
| 32 | CHEMBL482194 | N[C@@H]1CCN(C1)C(═O)[C@H] | [(3R)-3-Amino-1 |
| | | 1CCCCN1S(═O)(═O)c1ccc(cc1)c1ccccc1 | pyrrolidinyl][(2R)-1-(4- |
| | | | biphenylylsulfonyl)-2 |
| | | | piperidinyl]methanone |
| 33 | CHEMBL523100 | O═C(N1CCN(CC1)S(═O)(═O) | Cyclohexyl[4-(1- |
| | | c1cccc2c1cccc2)C1CCCCC1 | naphthylsulfonyl)-1- |
| | | | piperazinyl]methanone |
| 34 | CHEMBL525204 | O═C(N1CCN(CC1(C)C)S(═O)(═O) | Cyclohexyl[2,2-dimethyl-4-(1- |
| | | c1cccc2c1cccc2)C1CCCCC1 | naphthylsulfonyl)-1- |
| | | | piperazinyl]methanone |

TABLE 10
Specific examples of ADME properties of the selected candidates (Dataset 2)
obtained in SwissADME (part 1); in specific examples, Compounds 10, 11, 17, and 18 appeared as
good drug candidates, as they are predicted do not inhibit cytochromes P450 isoforms.

| Compound | | | | | | | | | |
|---|---|---|---|---|---|---|---|---|---|
| # | 10 | 11 | 12 | 13 | 14 | 15 | 16 | 17 | 18 |
| MW | 433.52 | 445.58 | 407.53 | 415.51 | 449.95 | 463.98 | 494.65 | 434.53 | 432.54 |
| #H-bond | 6 | 5 | 5 | 5 | 5 | 5 | 5 | 5 | 6 |
| acceptors | | | | | | | | | |
| #H-bond | 4 | 4 | 2 | 1 | 1 | 0 | 2 | 4 | 5 |
| donors | | | | | | | | | |
| TPSA | 145.99 | 136.76 | 103.96 | 95.17 | 95.17 | 86.38 | 95.09 | 148.16 | 148.79 |
| Consensus | 1.04 | 2.12 | 2.16 | 1.91 | 2.43 | 2.58 | 3.55 | −0.72 | 0.56 |
| Log P | | | | | | | | | |
| Ali Class | Soluble | Moderately | Moderately | Soluble | Moderately | Moderately | Moderately | Soluble | Soluble |
| | | soluble | soluble | | soluble | soluble | soluble | | |
| GI | Low | Low | High | High | High | High | High | Low | Low |
| absorption | | | | | | | | | |
| BBB | No | No | No | No | No | No | No | No | No |
| permeant | | | | | | | | | |
| CYP1A2 | No | No | No | No | No | No | No | No | No |
| inhibitor | | | | | | | | | |
| CYP2C19 | No | No | Yes | Yes | Yes | Yes | Yes | No | No |
| inhibitor | | | | | | | | | |
| CYP2C9 | No | No | No | Yes | Yes | Yes | No | No | No |
| inhibitor | | | | | | | | | |
| CYP2D6 | No | No | No | No | Yes | Yes | Yes | No | No |
| inhibitor | | | | | | | | | |
| CYP3A4 | No | No | Yes | Yes | Yes | Yes | Yes | No | No |
| inhibitor | | | | | | | | | |

TABLE 11
Specific examples of ADME properties of the selected candidates (Dataset 2)
obtained in SwissADME (part 2). In specific examples, Compounds 19, 22 and 23 appeared as a
good drug candidates, as they are predicted do not inhibit cytochromes P450 isoforms.

| Compound # | 19 | 20 | 21 | 22 | 23 | 24 | 25 | 26 | 27 | 28 |
|---|---|---|---|---|---|---|---|---|---|---|
| MW | 432.56 | 512.62 | 495.59 | 450.57 | 446.59 | 494.49 | 440.52 | 451.5 | 494.49 | 517.02 |
| #H-bond | 4 | 6 | 6 | 5 | 4 | 9 | 6 | 7 | 9 | 8 |
| acceptors | | | | | | | | | | |
| #H-bond | 4 | 0 | 0 | 3 | 4 | 2 | 2 | 2 | 2 | 3 |
| donors | | | | | | | | | | |
| TPSA | 138.93 | 106.69 | 103.45 | 147.21 | 138.93 | 141.74 | 141.74 | 165.53 | 141.74 | 158.51 |
| Consensus | 0.26 | 2.02 | 2.38 | −0.34 | 0.84 | 2.24 | 1.53 | 1.06 | 2.19 | 1.08 |
| Log P | | | | | | | | | | |
| Ali Class | Moderately | Moderately | Moderately | Soluble | Moderately | Moderately | Moderately | Moderately | Moderately | Moderately |
| | soluble | soluble | soluble | | soluble | soluble | soluble | soluble | soluble | soluble |
| GI | High | High | High | Low | High | Low | Low | Low | Low | Low |
| absorption | | | | | | | | | | |
| BBB | No | No | No | No | No | No | No | No | No | No |
| permeant | | | | | | | | | | |
| CYP1A2 | No | No | No | No | No | No | No | No | No | No |
| inhibitor | | | | | | | | | | |
| CYP2C19 | No | No | Yes | No | No | Yes | No | No | Yes | No |
| inhibitor | | | | | | | | | | |
| CYP2C9 | No | No | Yes | No | No | No | No | No | No | No |
| inhibitor | | | | | | | | | | |
| CYP2D6 | No | No | No | No | No | Yes | No | No | Yes | No |
| inhibitor | | | | | | | | | | |
| CYP3A4 | No | Yes | Yes | No | No | Yes | Yes | Yes | Yes | Yes |
| inhibitor | | | | | | | | | | |

TABLE 12
Specific examples of ADME properties of the selected candidates (Dataset 2)
obtained in SwissADME (part 3).

| Compound # | 29 | 30 | 31 | 32 | 33 | 34 |
|---|---|---|---|---|---|---|
| MW | 491.64 | 469.64 | 495.59 | 413.53 | 386.51 | 414.56 |
| #H-bond | 5 | 5 | 6 | 5 | 4 | 4 |
| acceptors | | | | | | |
| #H-bond donors | 0 | 1 | 0 | 1 | 0 | 0 |
| TPSA | 69.31 | 78.1 | 103.45 | 92.09 | 66.07 | 66.07 |
| Consensus Log | 3.56 | 3.44 | 2.36 | 2.26 | 3.05 | 3.6 |
| P | | | | | | |
| Ali Class | Moderately | Moderately | Moderately | Soluble | Moderately | Moderately |
| | soluble | soluble | soluble | | soluble | soluble |
| GI absorption | High | High | High | High | High | High |
| BBB permeant | Yes | No | No | No | Yes | Yes |
| CYP1A2 | No | No | No | No | Yes | No |
| inhibitor | | | | | | |
| CYP2C19 | Yes | Yes | Yes | No | Yes | Yes |
| inhibitor | | | | | | |
| CYP2C9 | Yes | Yes | Yes | No | Yes | Yes |
| inhibitor | | | | | | |
| CYP2D6 | Yes | Yes | No | Yes | Yes | Yes |
| inhibitor | | | | | | |
| CYP3A4 | Yes | Yes | Yes | Yes | Yes | Yes |
| inhibitor | | | | | | |

Embodiments can additionally or alternatively include applying any suitable approaches described herein for identification, generation, application, provision, and/or other suitable usage (e.g., in therapeutic compositions, etc.) of any suitable drugs, peptides, proteins, and/or other components, such as for use to prevent attachment and/or cleavage mediated by H.pylori, and/or thus they can be used as palliative and/or as a treatment against gastric cancer and/or any other suitable gastrointestinal conditions, cancers, and/or other suitable conditions.

In embodiments, the method and/or system can include and/or otherwise function to determine, generate, provide, and/or otherwise facilitate small molecules (e.g., peptides; in form of therapeutic composition(s); etc.) which inhibit the interaction between CagA and E-cadherin (e.g., such as for diagnostics and/or treatment; etc.), which include one of the routes reported for the GC development and/or other suitable conditions.

In embodiments, the method and/or system can include and/or otherwise function to inhibit (e.g., through small molecules, drugs, therapeutic compositions; etc.) the pathway involving binding of CagA (from H.pylori) and human E-cadherin, which has been described as one of the pathways that induce gastric cancer and/or other suitable conditions.

In embodiments, the method and/or system can include and/or otherwise function to design, determine, generate, provide, and/or otherwise facilitate new peptide-like drugs that can inhibit CagA/E-cadherin interaction (e.g., Since CagA interaction with E-cadherin provokes an abnormal interaction between E-cadherin and β-catenin).

In embodiments, the method and/or system can include and/or otherwise function to determine, generate, provide, and/or otherwise facilitate peptide-like drugs that can be used for treatment and/or as a palliative drug, such as additionally or alternatively with other treatments against H. pylori, Gastric Cancer, and/or other suitable conditions.

In specific examples, the method and/or system can include a first stage (and/or performed at any time and/or frequency; etc.), which can include determining the sequence(s) of the peptide(s). In specific examples, then (and/or at any suitable time and frequency) in vitro and/or in vivo assays can be used for testing; resulting formulations of peptides and/or other suitable molecules can be used for gastric cancer diagnostics and/or treatment.

### Specific Examples—Molecular Structures

First (and/or performed at any time and/or frequency; etc.), a molecular structure of ID (intracellular domain) can be modeled (e.g., homology model; etc.) using as template the crystallographic structure of E-cadherin of Mus musculus (PDB code: 1I7X), and the subsequence of P12830 from Uniprot as target; however, any suitable molecules and/or components can be used as the template and target.

>sp|P12830|731-882
LRRRAVVKEPLLPPEDDTRDNVYYYDEEGGGEEDQDFDLSQLHRGLDARP
EVTRNDVAPTLMSVPRYLPRPANPDEIGNFIDENLKAADTDPTAPPYDSL
LVFDYEGSGSEAASLSSLNSSESDKDQDYDYLNEWGNRFKKLADMYGGGE
DD

The crystal structure of bacterial virulence protein CagA-CM (particularly the CM domain of CagA) can be obtained from Protein Data Bank (PDB code: 3EIC). However, any suitable databases can be used, and/or any suitable templates (e.g., suitable regions, suitable strains, etc.) can be used.

### Specific Examples—Molecular Docking

A molecular docking can be performed to model the interaction between E-cadherin and CagA at the atomic level, such as for use in characterizing the particular binding site, of the interaction of CagA-CM in a non-specific location of CD domain protein. In a specific example, the docking showed a clear in region corresponding to “DTDPTAPPYDSL” peptide.

However, any suitable docking characterization approaches and/or suitable in silico approaches and/or other suitable approaches can be performed for modeling interaction and/or for other suitable purposes.

### Specific Examples—Reengineering

Taking into account the above (and/or suitable approaches described herein), embodiments of the method and/or system can include designing inhibitors of Helicobacter pylori, such as to abolish the GC cell growth and/or oncogenic responses (e.g., based on the inhibition of Citotoxin gen A (CagA); etc.). In examples, the method and/or system can include reengineering of “DTDPTAPPYDSL” inhibitory peptide (and/or other suitable selected peptides, such as based on molecular docking characterization(s); etc.) using a docking method approach; but any suitable approach can be used for reengineering. The reengineering can include mutating any combination of and/or each position of “DTDPTAPPYDSL” peptide for the 19 (and/or other suitable number of) amino acids remaining. Thus, a control docking between the control peptide and CagA can be performed. In a specific example, the control docking resulted in a binding energy of −4.0 kcal/mol. Docking between reengineered peptides and CagA was performed. Examples of Results are described in the next table, highlighting the most favorable substitutions:

### Embodiments can Additionally or Alternatively Include:

inhibitors of CagA-H. pylori, which are based on peptide DTDPTAPPYDSL, where:

### Position 1: R,H,I,F,P,W,Y

### Position 2: N

### Position 3: N,Y

### Position 4: E,L,Y

### Position 5: R,L

### Position 6: S

### Position 7:R,E,I,L

### Position 8: I,LW

### Position 9: F

### Position 10: F,W

### Position 11: A,D,E,H,I,L,Y

### Position 12: A,N,W,Y.

In some embodiment, the CagA inhibitor has the sequence of X₁X₂X₃X₄X₅X₆X₇X₈X₉X₁₀X₁₁X₁₂; wherein:
X₁ is D, R, H, I, F, P, W, or Y;
X₂ is T or N;
X₃ is D, N, or Y;
X₄ is P, E, L, or Y;
X₅ is T, R, or L;
X₆ is A or S;
X₇ is P, R, E, I, or L;
X₈ is P, I, L, or W;
X₉ is F, or Y;
X₁₀ is D, F, or W;
X₁₁ is S, A, D, E, H, I, L, or Y; and
X₁₂ is L, A, N, W, or Y.

Embodiments of the present disclosure include peptides having at least 80%, 85%, 90%, 92%, 95%, 97%, 98%, 99% or 100% identity, to the sequence of SEQ ID NO: 1-38, listed in Table 13. Embodiments of the present disclosure also include peptides having non-natural amino acid.

TABLE 3
peptide sequences

| SEQ ID No | Sequence | Binding energy |
|---|---|---|
| 1 | DTDPTAPPYDSL | −4.0 |
| 2 | RTDPTAPPYDSL | −4.3 |
| 3 | HTDPTAPPYDSL | −4.4 |
| 4 | IDTDPTAPPYDSL | −4.3 |
| 5 | FTDPTAPPYDSL | −4.3 |
| 6 | PTDPTAPPYDSL | −4.3 |
| 7 | WTDPTAPPYDSL | −4.3 |
| 8 | YTDPTAPPYDSL | −4.7 |
| 9 | DNDPTAPPYDSL | −4.5 |
| 10 | DTNPTAPPYDSL | −4.2 |
| 11 | DTYPTAPPYDSL | −4.2 |
| 12 | DTDEAPPYDSL | −4.3 |
| 13 | DTDLTAPPYDSL | −4.3 |
| 14 | DTDYTAPPYDSL | −4.5 |
| 15 | DTDPRAPPYDSL | −4.3 |
| 16 | DTDPLAPPYDSL | −4.3 |
| 17 | DTDPTSPPYDSL | −4.3 |
| 18 | DTDPTARPYDSL | −4.3 |
| 19 | DTDPTAEPYDSL | −4.3 |
| 20 | DTDPTAIPYDSL | −4.4 |
| 21 | DTDPTALPYDSL | −4.3 |
| 22 | DTDPTAPIYDSL | −4.4 |
| 23 | DTDPTAPLYDSL | −4.4 |
| 24 | DTDPTAPWYDSL | −4.3 |
| 25 | DTDPTAPPFDSL | −4.4 |
| 26 | DTDPTAPPYFSL | −4.5 |
| 27 | DTDPTAPPYWSL | −4.3 |
| 28 | DTDPTAPPYDAL | −4.3 |
| 29 | DTDPTAPPYDDL | −4.4 |
| 30 | DTDPTAPPYDEL | −4.3 |
| 31 | DTDPTAPPYDHL | −4.3 |
| 32 | DTDPTAPPYDIL | −4.4 |
| 33 | DTDPTAPPYDLL | −4.3 |
| 34 | DTDPTAPPYDYL | −4.7 |
| 35 | DTDPTAPPYDSA | −4.2 |
| 36 | DTDPTAPPYDSN | −4.2 |
| 37 | DTDPTAPPYDSW | −4.2 |
| 38 | DTDPTAPPYDSY | −4.2 |

However, any suitable substitutions can be made, such as for improving binding energy metrics.

Embodiments of the method and/or system can additionally or alternatively include:
The use of the aforementioned inhibitors (and/or suitable molecules described herein, such as molecules derivable from approaches described herein; etc.) to diagnose and/or treat GC and/or a health condition wherein H. pylori is involved, and/or any suitable conditions.
The use of the specific inhibitors described herein (and/or derivable from approaches described herein; etc.) to diagnose and/or treat GC and/or a health condition wherein H. pylori is involved, and/or any suitable conditions (e.g., gastrointestinal conditions; cancer conditions; etc.).
A methodology to obtain inhibitors of a protein, wherein the methodology comprises a modeling of the protein, identifying binding sites to perform docking of molecules (e.g peptides), and/or selecting those molecules as best binders to the modeled protein.
One or more therapeutic compositions based on, including, and/or otherwise associated with one or more peptides, inhibitors, binding sites, and/or molecules described herein, such as for facilitating diagnosis and/or therapeutic intervention for GC, conditions associated with H. pylori, and/or any suitable conditions (e.g., gastrointestinal conditions; cancer conditions; etc.).

1. A formulation in pharmaceutically acceptable form suitable for administration to a patient containing an excipient and a HtrA inhibitor having Formula (I): 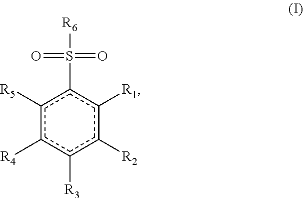
wherein:
R₁ is H, halo, cyano, OH, (C₁-C₆)alkyl, (C₃-C₁₀)heterocyclo, (C₃-C₁₀)cycloalkyl, aryl, or heteroaryl, any of which may be optionally substituted with 1 or more R^{w} groups as allowed by valence selected from the groups listed in Table 1;
R₂ is H, halo, cyano, OH, (C₁-C₆)alkyl, (C₂-C₈)alkenyl, (C₁-C₆)alkoxy, (C₃-C₁₀)heterocyclo, (C₃-C₁₀)cycloalkyl, aryl, or heteroaryl, any of which may be optionally substituted with 1 or more R^{w} groups as allowed by valence;
R₃ is H, halo, cyano, OH, (C₁-C₆)alkyl, (C₂-C₈)alkenyl, (C₂-C₈)carboxyalkyl; N—(C₁-C₆)alkylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence;
R₄ is H, halo, cyano, OH, (C₁-C₆)alkyl, (C₂-C₈)alkenyl, (C₂-C₈)carboxyalkyl; N—(C₁-C₆)alkylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence;
R₅ is H, halo, cyano, OH, (C₁-C₆)alkyl, (C₂-C₈)alkenyl, (C₂-C₈)carboxyalkyl; N—(C₁-C₆)alkylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence;
R₆ is H, halo, cyano, OH, (C₁-C₆)alkyl, (C₂-C₈)alkenyl, (C₂-C₈)carboxyalkyl; N—(C₁-C₆)alkylaminocarbonyl, (C₁-C₆)alkoxy, (C₁-C₆)sulfonyl, (C₃-C₁₀)heterocyclo, (C₃-C₁₀)cycloalkyl, aryl, or heteroaryl, any of which may be optionally substituted with 1 or more R^{w} groups as allowed by valence;
R^{w} at each occurrence is independently H, halo, cyano, nitro, oxo, amino, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl, wherein n is 0, 1, 2, 3, 4, 5, or 6.

2. The formulation of claim 1, wherein R₁ is selected from the groups listed in Table 1; R₂ is selected from the groups listed in Table 2; R₃ is selected from the groups listed in Table 3; R₄ is selected from the groups listed in Table 4; R₅ is selected from the groups listed in Table 5; and R₆ is selected from the groups listed in Table 6.

3. The formulation of claim 1, wherein the HtrA inhibitor is selected from the group consisting of:
N′-benzyl-N-[2-[(2R)-1-(4-methylphenyl)sulfonylpiperidin-2-yl]ethyl]oxamide;
1-[2-[methyl(naphthalen-2-ylsulfonyl)amino]acetyl]piperidine-4-carboxamide;
8-(4-methylphenyl)sulfonyl-4-(2,4,6-trimethylphenyl)sulfonyl-1-oxa-4,8-diazaspiro[4.5]decane;
N-{1-[2-(2-Biphenylyloxy)ethyl]-3-pyrrolidinyl}-3,4-difluorobenzenesulfonamide;
1-(2-Anthrylsulfonyl)-3-piperidinecarboxylic acid;
(3S)-1-Carbamimidoyl-N-({(2S)-1-[N-(2-naphthylsulfonyl)glycyl]-2-pyrrolidinyl}methyl)-3-piperidinecarboxamide;
(3S)-1-Carbamimidoyl-N-({(2S)-1-[N-(2-naphthylsulfonyl)-L-alanyl]-2-pyrrolidinyl}methyl)-3-piperidinecarboxamide;
Cyclohexyl[4-(1-naphthylsulfonyl)-2-(trifluoromethyl)-1-piperazinyl]methanone; and
2-[(8S,11R)-11-{(1R)-1-hydroxy-2-[(3-methylbutyl)(phenylsulfonyl)amino]ethyl}-6,9-dioxo-2-oxa-7,10-diazabicyclo[11.2.2]heptadeca-1(15),13,16-trien-8-yl]acetamide.

4. A method of treating a bacterial infection comprising administering a pharmaceutically effective amount of the HtrA inhibitor of claim 1 to a human subject in need thereof.

5. The method of claim 4, wherein the bacterial infection is a Helicobacter pylori infection.

6. A CagA inhibitor having the sequence of X₁X₂X₃X₄X₅X₆X₇X₈X₉X₁₀X₁₁X₁₂; wherein:
X₁ is D, R, H, I, F, P, W, or Y;
X₂ is T or N;
X₃ is D, N, or Y;
X₄ is P, E, L, or Y;
X₅ is T, R, or L;
X₆ is A or S;
X₇ is P, R, E, I, or L;
X₈ is P, I, L, or W;
X₉ is F, or Y;
X₁₀ is D, F, or W;
X₁₁ is S, A, D, E, H, I, L, or Y; and
X₁₂ is L, A, N, W, or Y.

7. The CagA inhibitor of claim 6, having the sequence of DTDPTAPPYDSL.

8. The CagA inhibitor of claim 6, having the sequence listed in Table 13.

9. A CagA inhibitor having the sequence at least 80% identity to any of the sequence of SEQ ID NOs: 1-38, listed in Table 13.

10. A method of treating gastric cancer comprising administering a pharmaceutically effective amount of the CagA inhibitor of claim 6 to a human subject in need thereof.

11. A method of inhibiting, down-regulating, reducing and/or killing pathogenic bacteria comprising steps of:
a. Screening a microorganism that produces an antibacterial compound;
b. Conducting structural analysis of the antibacterial compound;
c. Modifying the antibacterial compound to improve the affinity to target bacteria.

12. The method of claim 11, wherein BLAST, FASTA or CLUSTAL is used for sequence analysis in step a).

13. The method of claim 11, wherein at least one amino acid of the antibacterial compound is mutated in step c).

14. The method of claim 11, where the structural analysis is performed using solvent-accessible surface area.

15. The method of claim 11, wherein the antibacterial compound is Salivaricin A, Ruminococcin A, or Bacteriocin staphylococcus 188.


SEQUENCE LISTING (39 sequences)

SEQ ID NO: 1 - protein, 12 aa, Artificial Sequence
  Synthetic Sequence
DTDPTAPPYDSL

SEQ ID NO: 2 - protein, 12 aa, Artificial Sequence
  Synthetic Sequence
RTDPTAPPYDSL

SEQ ID NO: 3 - protein, 12 aa, Artificial Sequence
  Synthetic Sequence
HTDPTAPPYDSL

SEQ ID NO: 4 - protein, 13 aa, Artificial Sequence
  Synthetic Sequence
IDTDPTAPPYDSL

SEQ ID NO: 5 - protein, 12 aa, Artificial Sequence
  Synthetic Sequence
FTDPTAPPYDSL

SEQ ID NO: 6 - protein, 12 aa, Artificial Sequence
  Synthetic Sequence
PTDPTAPPYDSL

SEQ ID NO: 7 - protein, 12 aa, Artificial Sequence
  Synthetic Sequence
WTDPTAPPYDSL

SEQ ID NO: 8 - protein, 12 aa, Artificial Sequence
  Synthetic Sequence
YTDPTAPPYDSL

SEQ ID NO: 9 - protein, 12 aa, Artificial Sequence
  Synthetic Sequence
DNDPTAPPYDSL

SEQ ID NO: 10 - protein, 12 aa, Artificial Sequence
  Synthetic Sequence
DTNPTAPPYDSL

SEQ ID NO: 11 - protein, 12 aa, Artificial Sequence
  Synthetic Sequence
DTYPTAPPYDSL

SEQ ID NO: 12 - protein, 11 aa, Artificial Sequence
  Synthetic Sequence
DTDEAPPYDSL

SEQ ID NO: 13 - protein, 12 aa, Artificial Sequence
  Synthetic Sequence
DTDLTAPPYDSL

SEQ ID NO: 14 - protein, 12 aa, Artificial Sequence
  Synthetic Sequence
DTDYTAPPYDSL

SEQ ID NO: 15 - protein, 12 aa, Artificial Sequence
  Synthetic Sequence
DTDPRAPPYDSL

SEQ ID NO: 16 - protein, 12 aa, Artificial Sequence
  Synthetic Sequence
DTDPLAPPYDSL

SEQ ID NO: 17 - protein, 12 aa, Artificial Sequence
  Synthetic Sequence
DTDPTSPPYDSL

SEQ ID NO: 18 - protein, 12 aa, Artificial Sequence
  Synthetic Sequence
DTDPTARPYDSL

SEQ ID NO: 19 - protein, 12 aa, Artificial Sequence
  Synthetic Sequence
DTDPTAEPYDSL

SEQ ID NO: 20 - protein, 12 aa, Artificial Sequence
  Synthetic Sequence
DTDPTAIPYDSL

SEQ ID NO: 21 - protein, 12 aa, Artificial Sequence
  Synthetic Sequence
DTDPTALPYDSL

SEQ ID NO: 22 - protein, 12 aa, Artificial Sequence
  Synthetic Sequence
DTDPTAPIYDSL

SEQ ID NO: 23 - protein, 12 aa, Artificial Sequence
  Synthetic Sequence
DTDPTAPLYDSL

SEQ ID NO: 24 - protein, 12 aa, Artificial Sequence
  Synthetic Sequence
DTDPTAPWYDSL

SEQ ID NO: 25 - protein, 12 aa, Artificial Sequence
  Synthetic Sequence
DTDPTAPPFDSL

SEQ ID NO: 26 - protein, 12 aa, Artificial Sequence
  Synthetic Sequence
DTDPTAPPYFSL

SEQ ID NO: 27 - protein, 12 aa, Artificial Sequence
  Synthetic Sequence
DTDPTAPPYWSL

SEQ ID NO: 28 - protein, 12 aa, Artificial Sequence
  Synthetic Sequence
DTDPTAPPYDAL

SEQ ID NO: 29 - protein, 12 aa, Artificial Sequence
  Synthetic Sequence
DTDPTAPPYDDL

SEQ ID NO: 30 - protein, 12 aa, Artificial Sequence
  Synthetic Sequence
DTDPTAPPYDEL

SEQ ID NO: 31 - protein, 12 aa, Artificial Sequence
  Synthetic Sequence
DTDPTAPPYDHL

SEQ ID NO: 32 - protein, 12 aa, Artificial Sequence
  Synthetic Sequence
DTDPTAPPYDIL

SEQ ID NO: 33 - protein, 12 aa, Artificial Sequence
  Synthetic Sequence
DTDPTAPPYDLL

SEQ ID NO: 34 - protein, 12 aa, Artificial Sequence
  Synthetic Sequence
DTDPTAPPYDYL

SEQ ID NO: 35 - protein, 12 aa, Artificial Sequence
  Synthetic Sequence
DTDPTAPPYDSA

SEQ ID NO: 36 - protein, 12 aa, Artificial Sequence
  Synthetic Sequence
DTDPTAPPYDSN

SEQ ID NO: 37 - protein, 12 aa, Artificial Sequence
  Synthetic Sequence
DTDPTAPPYDSW

SEQ ID NO: 38 - protein, 12 aa, Artificial Sequence
  Synthetic Sequence
DTDPTAPPYDSY

SEQ ID NO: 39 - protein, 152 aa, Artificial Sequence
  Synthetic Sequence
LRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD